(19) United States
(12) Patent Application Publication
(10) Pub. No.: US 2026/0124319 A1
(43) Pub. Date: May 7, 2026

(54) NANOPARTICLES COMPRISING P52K PROTEIN AND ONE OR MORE NUCLEIC ACIDS AND, OR PROTEINS OF INTEREST AND METHODS OF USE THEREOF FOR DELIVERY TO CELLS

(71) Applicant: THE CHILDREN'S HOSPITAL OF PHILADELPHIA, Philadelphia, PA (US)

(72) Inventors: Matthew J. CHARMAN, Philadelphia, PA (US); Matthew D. WEITZMAN, Bala Cywyd, PA (US)

(73) Assignee: THE CHILDREN'S HOSPITAL OF PHILADELPHIA, Philadelphia, PA (US)

(21) Appl. No.: 18/997,705

(22) PCT Filed: Jul. 24, 2023
(86) PCT No.: PCT/US2023/028473

§ 371 Date: Jan. 22, 2025

Related U.S. Application Data
(60) Provisional application No. 63/391,374, filed on Jul. 22, 2022.

(51) Int. Cl. A61K 48/00; C07K 19/00

(52) U.S. Cl. CPC A61K 48/0033; C07K 19/00

(57) ABSTRACT

Nanoparticles comprising p52K and nucleic acids and/or proteins of interest which self-assemble to form a p52K nucleic acid and/or p52K protein complexes are disclosed. The nanoparticles find use, for example, in the delivery of the aforementioned nucleic acids and or proteins into cells using a pharmaceutically acceptable carrier.

### CROSS-REFERENCE TO RELATED APPLICATION

This application is a § 371 of International Application No. PCT/US2023/028473, filed Jul. 24, 2023, which claims priority to U.S. Provisional Patent Application No. 63/391,374 filed Jul. 22, 2022, the entire contents of each being incorporated herein by reference.

### GRANT STATEMENT

This invention was made with government support under grant numbers R01-AI145266, R01-AI121321 and R01-AI118891 awarded by the National Institute of Allergy and Infectious Diseases. The government has certain rights in the invention.

### INCORPORATION BY REFERENCE OF MATERIAL SUBMITTED IN ELECTRONIC FORM

The Contents of the electronic sequence listing (CHOP-139-US01.xml; Size: 33,040 bytes; and Date of Creation: Jan. 5, 2026) is herein incorporated by reference in its entirety.

### FIELD OF THE INVENTION

This invention relates to the fields of nanoparticle production and delivery of nucleic acids encoding proteins of interest. Specifically, the invention provides nanoparticles comprising p52K, and optionally, other accessory or viral proteins, which self-assemble upon binding to nucleic acids, forming a p52K nucleic acid complex. The nanoparticles find use, for example in the delivery of the nucleic acids and optionally other proteins into cells.

### BACKGROUND OF THE INVENTION

Several publications and patent documents are cited throughout the specification in order to describe the state of the art to which this invention pertains. Each of these citations is incorporated herein by reference as though set forth in full.

Biomolecular condensates (BMCs) formed by liquid-liquid phase-separation (LLPS) play fundamental roles in compartmentalizing and regulating cellular processes^{1,2}. During infection, viruses hijack host cells as they replicate and ultimately package genomes into progeny particles for viral spread and transmission. The emerging importance of BMCs as a way of organizing and regulating cellular processes has led to suggestions that viral membrane-less sub-cellular compartments are BMCs, with evidence presented for and against LLPS as the driver of viral BMC formation^{3-5,7,10}. Particular attention has been focused on RNA viruses which replicate in the cytoplasm, such as SARS-CoV-2 and respiratory syncytial virus (RSV)^{3,4,6,8,11}. The importance of BMCs is supported by evidence that condensate hardening drugs can attenuate the spread of RSV infection in vivo⁶. The ability of specific purified viral proteins to undergo phase-separation in vitro further implicates BMCs in multiple different viral processes, including the compaction of viral RNA genomes that may facilitate packaging^{3,5,8,9,11}. However, progress towards demonstrating definitive mechanistic roles for phase-separation in virus infected cells has ultimately been hampered by the lack of tractable model systems in which phase-separation can be abolished and restored so that its contribution to productive infection can be determined.

### SUMMARY OF THE INVENTION

In accordance with the present invention, a nanoparticle formulation for delivery of at least one heterologous molecule of interest to a cell for expression and or production, therein is provided. An exemplary particle comprises self-assembling p52K protein or functional fragment thereof which forms a complex with said heterologous molecule of interest, forming a molecule-p52K complex, in a pharmaceutically acceptable carrier. In certain aspects, the nanoparticle formulation comprises a p52K protein encoded by a nucleic acid and comprises at least one ITR sequence at the 5′end, the 3′ end or both. In other aspects, the ITR region is present at both the 5′ and 3′ ends of the p52K. The heterologous molecule can be a nucleic acid of interest which can also comprise an ITR region present at the 5′end, the 3′ end or both ends. In certain aspects, the p52K protein comprises an IDR region and the N terminus, the C terminus or both, said IDR facilitating self-assembly of said nucleic acid-p52 complex. The nanoparticle formulations described above can also comprise viral proteins, such as protein VII. The nanoparticle can also contain one or more polypeptides having enzymatic, or metabolic functions.

In certain aspects, the nanoparticle formulation is encapsulated in a liposome or micelle. The liposome or micelle can further comprise targeting ligands on the surface thereof. The liposome or micelle can further comprise a hydrophobic group selected from the group consisting of lipophilic alkyl groups, cholesterol, and combinations thereof.

The at least one heterologous molecule of interest can include, without limitation, nucleic acid encoding a virus genome, a nucleic acid of interest encodes a metabolic gene, a nucleic acid encoding an inhibitory RNA molecule selected from an siRNA, an shRNA or a ribozyme, a nucleic acid encoding a guide RNA suitable for use with CRISPR Cas and a Cas9 enzyme, a nucleic acid modified to limit recognition by an innate immune response, a nucleic acid which expresses a chimeric antigen receptor and a nucleic acid of encoding a guide RNA which targets a site for single base editing and an enzyme suitable for use in base editing.

In other embodiments, the nanoparticle formulation comprises a nucleic acid encoding a protein which upon administration to a subject in need thereof, ameliorates symptoms for an indicated disorder listed in Table 2. Also provided are kits comprising the nanoparticle formulation described above, and instructions for use thereof.

A method of preparing a nanoparticle formulation of described above is also disclosed. An exemplary the method comprising: contacting a p52K protein or functional fragment thereof with at least one heterologous molecule of interest under conditions wherein a p52K protein-heterologous molecule containing complex forms, said complex being in a solution suitable for administration to a cell type of interest. Also provided is a method for preparation of a nanoparticle formulation comprising at least one heterologous molecule of interest selected from a nucleic acid of interest which encodes a virus genome, a nucleic acid of interest which encodes a metabolic gene, a nucleic acid of interest encoding an inhibitory RNA, an siRNA, an shRNA and a ribozyme, a nucleic acid of interest encoding a guide RNA suitable for use with CRISPR Cas and a Cas9 enzyme protein, a nucleic acid of interest is modified to limit recognition by an innate immune response, a nucleic acid of interest expresses a chimeric antigen receptor and a nucleic acid of interest said guide RNA targets a site for single base editing and an enzyme suitable for use in base editing. Methods for delivery of the nanoparticle formulations are also disclosed. Kits for practice of the foregoing methods also comprise an embodiment of the invention.

### BRIEF DESCRIPTION OF THE DRAWINGS

FIGS. 1A-1I. Viral structural proteins form nuclear bodies distinct from viral replication compartments. FIGS. 1A-I, Uninfected (Un.) or infected (AdV) human bronchial epithelial cells (hours post-infection, hpi). FIG. 1A, Accumulation of viral early and late proteins with tubulin as loading control. Size markers are indicated in kDa. FIG. 1B, Viral progeny production measured at indicated times as plaque forming units (PFU). Virus input (4 hpi) is shown (dashed line). FIG. 1C, Visualization of newly synthesized protein (+HPG) or negative control (−HPG). FIG. 1D, Mean nuclear fluorescence intensity (MNFI) of labelled protein. FIG. 1E, Newly synthesized protein at virus-induced structures termed viral late nuclear bodies (VLNBs) and viral replication compartments (VRCs). FIG. 1F, Co-localization of 52K, hexon trimers (hex), fiber trimers (fib), and cement protein IIIa. Line profile (dashed yellow line). FIG. 1G, Visualization of viral late nuclear bodies (52K) in relation to replication compartments (DBP). FIG. 1H, 52K localization phenotypes (0-IV). FIG. 1I, Quantification of phenotypes 0-IV. Image scale bars=10 μm. Three independent repeats plotted (FIG. 1B) or pooled (FIGS. 1D, 1I). Mean (columns or line) and standard deviation (error bars) shown. The number of nuclei analyzed is indicated (FIGS. 1D, 1I). ANOVA (FIG. 1B), or Brown-Forsythe and Welch's ANOVA with Dunnett's T3 tests (FIG. 1D). **** p<0.001.

FIGS. 2A-2G. Uninfected (Un.) or infected (AdV) human bronchial epithelial cells (hours post-infection, hpi). FIG. 2A, Visualization of newly synthesized protein enriched at viral late nuclear bodies (VLNBs) and replication compartments (VRCs) marked by 52K and DBP immunostaining, respectively. FIG. 2B, Maximum intensity projections of nuclei stained by DAPI. FIG. 2C, Nucleus size corresponding to (FIG. 2B). 3 repeats pooled, mean and standard deviation shown. FIG. 2D, Visualization of nuclear reorganization by immunostaining of laminA or histone H1. The nucleus is stained with DAPI. Line profile (dashed yellow line). FIG. 2E, Changes in nuclear morphology and VRC organization shown by DAPI staining and DBP immunostaining. Images correspond to nuclei presented in FIG. 1g. FIG. 2F, Co-localization of 52K, hexon trimers (hex), fiber trimers (fib), and cement protein IIIa. Line profile (dashed yellow line). FIG. 2G, Peripheral viral late nuclear body exhibiting discontinuous immunostaining of 52K. The nucleus is stained with DAPI. Mean (columns or line) and standard deviation (error bars) shown. The number of nuclei analyzed is indicated (FIG. 2C). Image scale bars=10 μm. Kruskal-Wallis test with Dunn's multiple comparisons (c). ns p>0.05, ** p<0.01, **** p<0.0001.

FIG. 3. Localization of indicated viral proteins in uninfected (Uninf.) or adenovirus (AdV) infected human bronchial epithelial cells at 22 hpi. Uninfected cells are shown as a negative control. Scale bar=10 μm. Outlines of nuclei are indicated (dotted white line).

FIGS. 4A-4E. Viral late nuclear bodies exhibit liquid-like behavior. Uninfected or infected doxycycline-inducible transgenic lung cells. FIG. 4A, Expression of trans genes (GFP, 52K-GFP), endogenous 52K, or GAPDH loading control with or without doxycycline (DOX). FIG. 4B, Localization of 52K-GFP and IIIa. FIG. 4C, 52K-GFP pre-bleach, post-bleach, or following recovery. FIG. 4D, Fluorescence recovery after photobleaching. Replicates (green lines) and mean (black line) are shown. Half-time of recovery (t_{half}) is indicated. FIG. 4E, Time series showing fusion of punctate VLNBs. Scale bars 5 μm (FIGS. 4B, 4C), or 1 μm (FIG. 4E). Dashed lines show the outline of nuclei (FIG. 4B).

FIGS. 5A-5F. FIGS. 5A-5D show uninfected or infected doxycycline-inducible transgenic lung cells. FIG. 5A, Localization of ectopically expressed GFP in relation to punctate or peripheral viral late nuclear bodies marked by immunostaining of IIIa. FIG. 5B, Fluorescence recovery of nuclear diffuse 52K-GFP in uninfected cells. Replicates (green lines) and mean (black line) are shown. Half-time of recovery (t_{half}) is indicated. FIG. 5C, Recovery of 52K-GFP fluorescence corresponding to (a) and FIG. 2c & 2d. FIG. 5D, Fusion events observed per cell during a 5-minute window of live cell imaging of 8 cells. FIGS. 5E-5F, Transient expression in 293T cells. FIG. 5E, Expression of GFP-tagged 52K full length (FL), or truncation mutants (Δ1-47, Δ1-119). FIG. 5F, Localization of GFP-tagged Δ1-119. Outline of the nucleus is shown (dotted line) (FIG. 5E). Mean (columns) and standard deviation (error bars) shown. Scale bars=10 μm. One-way ANOVA with Tukey's multiple comparisons (FIG. 5C). ns p>0.05, ** p<0.01, **** p<0.0001.

FIGS. 6A-6N. The 52K protein is necessary and sufficient for phase-separation which requires its intrinsically disordered region (IDR). FIG. 6A, Predicted disorder tendency of 52K. FIG. 6B, Schematic of full length (FL) 52K and truncated (Δ1-47, Δ1-119) mutants. FIG. 6C, Transient expression of GFP tagged FL 52K or Δ1-47 in 293T cells. FIG. 6D, Localization of transiently expressed GFP-tagged proteins. Low, intermediate, and high levels of expression indicated by mean nuclear fluorescence intensity (MNFI) are shown, and correlate with diffuse (Diff.), punctate (Punc.), or accumulated (Acc.) localization. FIG. 6E, Quantification of localization phenotypes. FIG. 6F, MNFI corresponding to each localization phenotype. FIG. 6G, In vitro phase separation of FL 52K or Δ1-47. FIG. 6H, Diagram representing phase separation of proteins at the indicated concentrations. Droplet-like condensates (circles), bunched condensates (bunches), or aggregates (clouds) are indicated. FIG. 6I-6N, Human bronchial epithelial cells infected with WT or Δ52K mutant adenovirus. FIG. 6I, Accumulation of viral early and late proteins, or loading controls (GAPDH, histone-H3). FIG. 6J, Accumulation of viral genomes. FIG. 6K, Punctate viral late nuclear bodies visualized by labelling of newly synthesized protein. 3 examples are indicated (arrows). FIG. 6L, Percentage of cells with punctate VLNBs. FIG. 6M, Diffuse (Diff.), punctate (Punct.) or peripheral (Periph.) localization phenotypes of cement protein IIIa. FIG. 6N, Percentage of cells exhibiting each IIIa localization phenotype. Three independent repeats plotted (FIGS. 6E, J, L, N) or pooled (FIG. 6F). Image scale bars=10 μm. Mean (columns or line) and standard deviation (error bars) shown. The number of nuclei analyzed is indicated (FIG. 6F). Unpaired t-tests (FIG. 6E), 2-way ANOVA with Tukey's (FIG. 6F), or Šidák's (FIG. 6J) tests. ns p>0.05, * p<0.05, ** p<0.01, *** p<0.001, **** p<0.0001.

FIGS. 7A-7D. Analysis of amino acid composition of the 52K protein. FIG. 7A, predictions of fold-index, fraction of charged residues (FCR), net charge per residue (NCPR), π-π interactions (Pi-Pi), and hydrophobicity. FIG. 7B, amino acid frequency of FL 52K, Δ1-47, or Δ1-119 compared to the mean frequency of proteins in the human proteome (Human) or protein phase separation database (PPS). FIG. 7C, amino acid frequency of the extreme N-terminal region (1-47) compared to the remaining predicted IDR (48-119), or remaining polypeptide (48-415). FIG. 7D, Schematic showing the N-terminal 119 amino acids of the 52K protein. The proline, alanine, and glutamine rich (PAQ) region corresponding to amino acids 1-47 is indicated.

FIGS. 8A-8D. FIG. 8A Cleavage of MBP tag from full length (FL) 52K, or truncation mutants Δ1-47, or Δ1-119 by addition of TEV protease at a ratio of 1:50 and incubation at 25° C. for 30 mins. FIG. 8B, In vitro phase-separation of FL 52K imaged after 10- or 120-minutes incubation.

FIG. 8C, In vitro phase-separation of 52K, Δ1-47, or Δ1-119 at indicated concentrations. FIG. 8D, purified viral genomic DNA (vDNA), with or without benzonase treatment. Image scale bars=10 μm.

FIGS. 9A-9K. Phase separation and the 52K protein PAQ region are essential for assembly of infectious particles containing viral genomes. FIG. 9A, In vitro phase-separation of 52K without (− vDNA), or with untreated (− Benzonase) or benzonase treated (+ Benzonase) viral DNA (+ vDNA). FIG. 9B, Phase-separation of full length (FL) 52K or truncation mutant Δ1-47 with or without vDNA. Condensates were imaged after 10 or 120 minutes of additional incubation. FIG. 9C, Confocal images of 52K protein condensates (green) at indicated vDNA concentrations. FIG. 9D, Condensate size corresponding to (FIG. 9C). Median (line), upper and lower quartiles (box), and 5-95 percentiles (whiskers) are shown. FIG. 9E, Number of condensates corresponding to (c). Mean and standard deviation are shown. FIG. 9F, Turbidity of 52K phase-separation assays at indicated vDNA concentrations. Uncleaved protein acts as a negative control (−TEV). FIGS. 9G-9J, Parental (Par.) or transgenic lung cells expressing FL 52K or truncation mutant (Δ1-47), infected with WT adenovirus or Δ52K mutant. FIG. 9G, Abundance of viral late proteins and 52K. FIG. 9H, Restoration of VLNBs indicated by 52K localization. FIG. 9I, Progeny production (focus forming units per mL, FFU/mL), virus input (4 hpi) is shown. FIG. 9J, Production of unpackaged or packaged viral particles. FIG. 9K, FIG. 9L, human bronchial epithelial cells incubated with or without 100 mM 1,6-hexanediol. FIG. 9K, VLNB formation. FIG. 9L, Viral progeny production (plaque forming units per mL, FFU/mL). Image scale bars=10 μm. Three independent repeats plotted (FIGS. 9E, 9F, 9I, 9L) or pooled (FIG. 9D). Mean (columns or line) and standard deviation (error bars) shown. Kruskal-Wallis ANOVA with Dunn's tests (FIG. 9D), one-way ANOVA with Šidák's (FIG. 9E), or Tukey's (FIG. 9I) tests, two-way ANOVA (FIG. 9F), or unpaired t-test (FIG. 9I). ns p>0.05, ** p<0.01, *** p<0.001, **** p<0.0001.

FIGS. 10A-10G. Effects of 1,6-hexanediol on infected (FIGS. 10A, 10D, 10E, 10F, 10G) or uninfected (FIGS. 10B, 10C) bronchial epithelial cells. FIG. 10A, Visualization of viral replication compartments (VRCs) or viral late nuclear bodies (VLNBs) marked by immunostaining of DBP or 52K respectively. Cells were incubated with or without 10% 1,6-hexanediol for 15 minutes prior to fixation at 22 hpi. The nucleus is stained with DAPI. FIG. 10B, Viability of live cells incubated with indicated concentrations of 1,6-hexanediol for 36 h. FIG. 10C, Visualization of coilin bodies in cells incubated with or without 100 mM 1,6-hexanediol for 36 h. Outline of the nucleus is shown (dotted white line). FIG. 10D, Visualization of VRCs at 16 hpi, in cells incubated with or without 100 mM 1,6-hexanediol from 2 hpi onwards. FIG. 10E, Accumulation of viral genomes. Cells incubated with or without 100 mM 1,6-hexanediol from 12 hpi onwards. FIG. 10F, Accumulation of viral proteins or GAPH loading control. Cells incubated with or without 100 mM 1,6-hexanediol from 12 hpi onwards. FIG. 10G, Viral progeny (PFU, plaque forming units) produced when cells were incubated with 100 mM 1,6-hexanediol following addition at the indicated times post-infection. Mean input virus (4 h) is shown as dotted line. Mean (columns or line) and standard deviation (error bars) shown. Image scale bars=10 μm. 2-way ANOVA (comparing 24, 36, and 48 hpi) with Šidák's multiple comparisons (FIG. 10E), or ANOVA with Dunnett's multiple comparisons (g). ns p>0.05, * p<0.05, *** p<0.001, **** p<0.0001.

FIG. 11. Top, When the 52K protein is present viral structural proteins form phase-separated BMCs. Modulation of BMCs by viral DNA genomes nucleates the assembly of packaged viral particles that mature into infectious particles. Bottom, In the absence of the 52K protein phase-separation does not occur and only empty non-infectious particles are assembled.

FIGS. 12A-12B. FIG. 12A, Coomassie gel shows purified MBP-52K and its cleavage by TEV protease (into MBP & 52K respectively). This is the process used to enable assembly. purified MBP-52K and its cleavage by TEV protease (into MBP & 52K respectively). This is the process used to enable assembly. FIG. 12B, Agarose gel showing purified viral genomes (vDNA). Benzonase treatment confirms that this is nucleic acid.

FIGS. 13A-13B. Self assembly of 52K+vDNA nanoscale particles. FIG. 13A and FIG. 13B show 2 schematics of the assembly process. MBP-52K and vDNA are combined in the same tube and TEV protease is added to release 52K from MBP and induce self-assembly of nanoscale particles.

FIGS. 14A-14C. Characterization of assembled nanoparticles. FIG. 14A. The MBP-52K used in the assembly was fluorescently labelled and visualized post-assembly by confocal microscopy. When high concentrations of vDNA are used the assembled particles generated are too small to visualize. FIG. 14B. These assemblies do however contribute to the turbidity of the solution (they scatter light). This tells us that although we can't see them by microscopy, they are present in the sample. FIG. 14C. These assemblies can also be crudely purified in bulk by centrifugation at 2000 g for 3 minutes. The pelleted assemblies can then be analyzed by SDS-PAGE and Coomassie stain.

FIGS. 15A-15F. FIGS. 15A and 15B show data generated by nanoparticle tracking analysis of nanoscale assemblies 4 hours post-assembly using a NANOSIGHT NS300 machine. No vDNA is included as a control. This type of analysis provides data on the size and number of particles in the sample. FIG. 15C shows analysis of nanoscale assemblies 4 hours post-assembly measuring Zeta-potential using the Malvern Zetasizer. No vDNA is included as a control in addition to empty viral particles (devoid of a genome) or packaged viral particles (containing GFP-encoding viral genome). This type of analysis determines the charge surrounding the particles. The increased negative charge of particles assembled in the presence of vDNA indicated incorporation of the negatively charged DNA. FIG. 15D shows another analysis of the Zeta-potential using the Malvern Zetasizer comparing particles assembled without nucleic acid, with vDNA, or with RNA. FIG. 15E shows particles assembled in all conditions. The fluorescence data indicates that particles assembled with vDNA can be detected by addition of RiboGreen, while particles assembled without vDNA are not. FIG. 15F shows that circular plasmid DNA can also be incorporated into particles. Nanoparticles were assembled without DNA, with vDNA, or with the circular plasmid pTG3602. RiboGreen was added to all conditions. NTA was then performed using two different modes. The first mode detects light scattering (as before), the second mode detects fluorescence. The light scattering data indicates that particles assembled in all conditions, with fewer particles assembled in the absence of DNA as in FIG. 15. The fluorescence data indicates that particles assembled with vDNA or circular DNA (pTG3602) and are detectable by addition of RiboGreen, while particles assembled without DNA are essentially undetectable. Thus, our particles assembled in the presence of vDNA and pTG3602 have incorporated DNA but the particles assembled without DNA have not.

FIGS. 16A-16F. Schematic showing representative examples of nucleic acid cargo for incorporation into nanoparticles. FIG. 16A. Adenovirus DNA genome. Flanking inverted terminal repeats (ITRs) are shown (orange). FIG. 16B. Linear DNA of interest without flanking regions. FIG. 16C. Linear DNA of interest including flanking regions (orange). FIG. 16D. Circular form of A-C. FIG. 16E. RNA of interest.

FIGS. 17A-17B. Size exclusion purification of nanoparticle. The Sephacryl resin provides a medium through which the sample flows by gravity. Larger particles move through the resin quickly and elute in earlier fractions, while individual proteins enter pores in the resin, which slows their progress so that they elute in later fractions. FIG. 17A. Fractions 1-26 were analyzed by western blot, immunoblotting for either 52K or maltose binding protein (MBP). Samples equivalent to 5% input was included for context. FIG. 17B. Fractions 1-26 were analyzed by quantitative PCR, using primer pairs specific to the viral DNA binding protein open reading frame present in the viral genome. The data demonstrate that 52K must be present in larger particles, as 52K elutes in fractions 9-11, while free MBP and fragments of 52K elute in fractions 17-23. The data also reveal that vDNA (detected by QPCR) also elutes in fractions 9-11 co-purifying with 52K indicating that both are in the same particles.

### DETAILED DESCRIPTION OF THE INVENTION

Phase-separated biomolecular condensates (BMCs) play fundamental roles in compartmentalizing and regulating many cellular processes. Although hypothesized to facilitate a number of viral processes, evidence that phase-separation contributes functionally to the assembly of infectious viral progeny in infected cells is lacking. Here we report the identification of phase-separated BMCs critical for the coordinated assembly of packaged progeny particles during adenovirus (AdV) infection. The data show that viral structural proteins accumulate in dynamic nuclear BMCs distinct from viral replication compartments which we term viral late nuclear bodies (VLNBs). Moreover, VLNB formation is driven by phase-separation of the viral 52K protein. Phase-separation of 52K requires arginine resides and polyampholytic distribution of charged amino acid residues within its N-terminal intrinsically disordered region. VLNBs function to sequester viral structural proteins to limit deleterious aggregation that would otherwise attenuate production of progeny particles, whilst maintaining a state that is conducive to coordinated assembly and packaging. The data also demonstrate that phase-separation of structural proteins is not alone sufficient to coordinate progeny production. We show that the 52K protein and viral genomes play a critical role in modulating VLNBs, leading to the assembly of packaged viral particles. Our findings demonstrate that both the formation and modulation of a viral BMC plays an essential role in the coordinated assembly of progeny particles containing packaged viral genomes.

This natural process has been exploited to facilitate encapsulation/complexation of heterologous nucleic acids of interest for expression and/or polypeptides of interest into non-infectious viral nanoparticles. Once formed, the particles are administered in suitable pharmaceutically acceptable carriers to subjects in need thereof to treat or ameliorate symptoms of a variety of different disorders and pathological conditions as described below.

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as Hawley's Condensed Chemical Dictionary 14th Edition, by R. J. Lewis, John Wiley & Sons, New York, N.Y., 2001.

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, the terms “including,” “includes,” “having,” “has,” “with,” or variants thereof, are intended to be inclusive similar to the term “comprising.”

The term “about” can refer to a variation of +/−5%, +/−10%, +/−20%, or +/−25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the end-points of a recited range as discuss above in this paragraph.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements.

As will also be understood by one skilled in the art, all language such as “up to,” “at least,” “greater than,” “less than,” “more than,” “or more,” and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents.

One skilled in the art will also readily recognize that where members are grouped together in a common manner, such as in a Markush group, the invention encompasses not only the entire group listed as a whole, but each member of the group individually and all possible subgroups of the main group.

As used herein “protein packaging protein 52K or p52K” refers to a protein involved in viral genome packaging through its interaction with packaging proteins 1 and 2. After proteolytic cleavage by adenovirus protease, L1 52/55k protein is removed from the capsid during viral maturation. The p52K is also known as “packaging protein 3” and “L1-52-55 kDA protein”. See Uniprot No. Q6VGV2 for the protein sequence. Also see Uniprot No. P04496. The protein comprises a N-terminal intrinsically disordered region (IDR) of approximately 119 amino acids. This region demonstrated several features which appear to contribute to protein phase-separation including local regions of net charge, a predicted tendency for π-π interactions, and small hydrophobic stretches. The IDR exhibits an amino acid composition bias, with arginine and proline, which are common in phase-separating proteins^{22,23}, over-represented compared to both the human proteome and full length (FL) 52K. Interestingly, the extreme N-terminal region of the IDR corresponding to amino acids 1-47, has a particularly high disorder tendency, contained a high proportion of proline, alanine, and glutamine and is designated as a proline, alanine and glutamine rich (PAQ) region.

As used herein, the term “adjuvant” refers to a compound that, with a specific immunogen or antigen, will augment or otherwise alter or modify the resultant immune response. Modification of the immune response includes intensification or broadening the specificity of either or both antibody and cellular immune responses. Modification of the immune response can also mean decreasing or suppressing certain antigen-specific immune responses. In certain embodiments, the adjuvant is a cyclic dinucleotide.

Certain components of the nanoparticle described herein are capable of self-assembly. As used herein, “noncovalently complexed” or “noncovalent complex” refers to the reversible association of two or more molecules. In certain embodiments, a noncovalent complex is formed through base-stacking interactions (e.g., pi-pi) and/or hydrogen-bonding. In certain embodiments, a noncovalent complex is formed between a nucleic acid of interest and an effective amount of p52K and optionally other accessory proteins or components.

As used herein, “a synthetic nanoparticle” refers to a self-assembled population of nucleic acids non-covalently complexed with p52K. In some embodiments, a nanoparticle has a diameter of less than 1000 nanometers (nm), less than 500 nm, less than 300 nm, or less than 200 nm. In some embodiments, a nanoparticle has a diameter of less than 100 nm. In some embodiments, a nanoparticle has a diameter in a range of between about 10 and 100 nm. In some embodiments, nanoparticles are micelles in that they comprise an enclosed compartment, separated from the bulk solution by a micellar membrane, comprised of amphiphilic entities which surround and enclose a space or compartment. In some embodiments, a nanoparticle has the structure of a worm-like micelle, a disk-like micelle, a nanofiber, a liposome or other type of spherical micelle.

The term lipid includes mono-, di- and triacylglycerols, phospholipids, free fatty acids, fatty alcohols, cholesterol, cholesterol esters, and the like. Liposomes are an important class of drug delivery vehicles that have been studied since the mid-1960s [1].

“Liposomes” serve as effective particulate drug carriers as they improve pharmacokinetic (PK) and pharmacodynamic (PD) profiles of small molecule drugs. In addition, remote loading methods can be used to improve the encapsulation efficiency, as in the example of Doxil: a liposomal formulation used in the setting of cancer. With the recent FDA approval of Onivyde (liposomal irinotican) and the previous approvals of Doxil, Depocyte, Daunoxome, and Ambisome, the liposome field has reached clinical utility. However, all of these drug preparations are non-targeted and are for the treatment of cancer. None of these formulations are actively targeted, but adding target-specific ligands to the surface of liposomes can further improve the therapeutic index of this new class of drugs. The liposomal surface can readily be modified by adding a wide variety of targeting ligands including antibodies, antibody fragments and peptides that have affinity for cell types and tissue components of interest. The targeting ligand provides for efficient accumulation of drugs in the tissue target of choice, thus reducing the drug exposure in non-target tissues.

The term “phospholipid” as used herein refers to a glycerol phosphate with an organic head-group such as choline, serine, ethanolamine or inositol and zero, one or two (typically one or two) fatty acids esterified to the glycerol backbone. Phospholipids include, but are not limited to, phosphatidylcholine, phosphatidylethanolamine, phosphatidylserine and phosphatidylinositol as well as corresponding lysophospholipids.

A “delivery vehicle” can mean the nanoparticle vehicle alone, or encapsulated in a liposome or micelle (optionally comprising targeting ligands or peptides for directing the vehicle to a target cell of interest), and/or one loaded with a therapeutic and/or a diagnostic agent.

As used herein, “self-assembling” refers to spontaneous or induced assembly of a molecule into defined, stable, noncovalently bonded assemblies that are held together by intermolecular forces. Self-assembling molecules formed from the p52K containing nanoparticle formulations described herein can include, for example protein, peptides, nucleic acids, virus-like particles, lipids and carbohydrates. In some embodiments, a PNA-amphiphile conjugate self-assembles via non-covalent interactions with an immunomodulatory compound, e.g., a cytokine, to form a synthetic nanoparticle.

As used herein, “vaccine” refers to a formulation which comprises a synthetic nanoparticle as described herein, combined with an antigen or a nucleic acid encoding said antigen, which is in a form that is capable of being administered to a vertebrate and which induces a protective immune response sufficient to induce immunity to prevent and/or ameliorate an infection or disease and/or to reduce at least one symptom of an infection or disease and/or to enhance the efficacy of another dose of the synthetic nanoparticle. Typically, the vaccine comprises a conventional saline or buffered aqueous solution medium in which a composition as described herein is suspended or dissolved. In this form, a composition as described herein is used to prevent, ameliorate, or otherwise treat an infection or disease. Upon introduction into a host, the vaccine provokes an immune response including, but not limited to, the production of antibodies and/or cytokines and/or the activation of cytotoxic T cells, antigen presenting cells, helper T cells, dendritic cells and/or other cellular responses.

In certain embodiments, the vaccine is a “cancer vaccine,” which refers to a treatment that induces the immune system to attack cells with one or more tumor associated antigens. The vaccine can treat existing cancer (e.g., therapeutic cancer vaccine) or prevent the development of cancer in certain individuals (e.g., prophylactic cancer vaccine). The vaccine creates memory cells that will recognize tumor cells with the antigen and therefore prevent tumor growth. In certain embodiments, the cancer vaccine comprises a synthetic nanoparticle, and a tumor-associated antigen.

As used herein, the term “immunogen” or “antigen” refers to a substance such as a protein, peptide, or nucleic acid that is capable of eliciting an immune response. Both terms also encompass epitopes, and are used interchangeably.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art, especially in light of the detailed disclosure provided herein. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

The terms “treating,” “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping or reversing the progression or severity of the condition or symptoms being treated. As such, the term “treatment” can include medical, therapeutic, and/or prophylactic administration, as appropriate.

The terms “inhibit,” “inhibiting,” and “inhibition” refer to the slowing, halting, or reversing the growth or progression of a disease, infection, condition, group of cells, protein or its expression. The inhibition can be greater than about 20%, 40%, 60%, 80%, 90%, 95%, or 99%, for example, compared to the growth or progression that occurs in the absence of the treatment or contacting.

A “targeting molecule” or “targeting agent” is a peptide or other molecule that binds to a targeted component). Optionally, the binding affinity of the targeting molecule may be in the range of 1 nM to 1 μM. In some embodiments, the targeting molecule may be an antagonist of a receptor on the surface of a targeted cell.

A “therapeutic agent,” “active agent,” or “drug” refers to any molecule used in the treatment, cure, prevention, or diagnosis of a disease or other medical condition. Examples of therapeutic agents include, but are not limited to, FDA-approved drugs, experimental drugs, antibiotics, and nucleic acids (e.g., siRNA, DNA).

The terms “active agent,” “therapeutic agent,” “drug,” and the like, are readily recognized by those of kill in the art. The micelles and liposomes described herein can encapsulate various drugs, such as those drugs exemplified in the description herein, or another therapeutic or otherwise active agent known in the art.

The terms “cell,” “cell line,” and “cell culture” as used herein may be used interchangeably. All of these terms also include their progeny, which are any and all subsequent generations. It is understood that all progeny may not be identical due to deliberate or inadvertent mutations.

A “coding region” of a gene consists of the nucleotide residues of the coding strand of the gene and the nucleotides of the non-coding strand of the gene which are homologous with or complementary to, respectively, the coding region of an mRNA molecule which is produced by transcription of the gene.

“Complementary” as used herein refers to the broad concept of subunit sequence complementarity between two nucleic acids, e.g., two DNA molecules. When a nucleotide position in both of the molecules is occupied by nucleotides normally capable of base pairing with each other, then the nucleic acids are considered to be complementary to each other at this position. Thus, two nucleic acids are complementary to each other when a substantial number (at least 50%) of corresponding positions in each of the molecules are occupied by nucleotides which normally base pair with each other (e.g., A:T and G:C nucleotide pairs). Thus, it is known that an adenine residue of a first nucleic acid region is capable of forming specific hydrogen bonds (“base pairing”) with a residue of a second nucleic acid region which is antiparallel to the first region if the residue is thymine or uracil. Similarly, it is known that a cytosine residue of a first nucleic acid strand is capable of base pairing with a residue of a second nucleic acid strand which is antiparallel to the first strand if the residue is guanine. A first region of a nucleic acid is complementary to a second region of the same or a different nucleic acid if, when the two regions are arranged in an antiparallel fashion, at least one nucleotide residue of the first region is capable of base pairing with a residue of the second region. In one embodiment, the first region comprises a first portion and the second region comprises a second portion, whereby, when the first and second portions are arranged in an antiparallel fashion, at least about 50%, including at least about 75%, at least about 90%, or at least about 95% of the nucleotide residues of the first portion are capable of base pairing with nucleotide residues in the second portion. In some embodiments, all nucleotide residues of the first portion are capable of base pairing with nucleotide residues in the second portion.

A “compound,” as used herein, refers to any type of substance or agent that is commonly considered a drug, or a candidate for use as a drug, as well as combinations and mixtures of the above.

A “control” cell is a cell having the same cell type as a test cell. The control cell may, for example, be examined at precisely or nearly the same time the test cell is examined. The control cell may also, for example, be examined at a time distant from the time at which the test cell is examined, and the results of the examination of the control cell may be recorded so that the recorded results may be compared with results obtained by examination of a test cell.

The use of the word “detect” and its grammatical variants refers to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

As used herein, an “effective amount” or “therapeutically effective amount” means an amount sufficient to produce a selected effect, such as alleviating symptoms of a disease or disorder. In the context of administering compounds in the form of a combination, such as multiple compounds, the amount of each compound, when administered in combination with another compound(s), may be different from when that compound is administered alone. Thus, an effective amount of a combination of compounds refers collectively to the combination as a whole, although the actual amounts of each compound may vary. The term “more effective” means that the selected effect is alleviated to a greater extent by one treatment relative to the second treatment to which it is being compared.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein, an “essentially pure” preparation of a particular protein or peptide is a preparation wherein at least about 95%, and preferably at least about 99%, by weight, of the protein or peptide in the preparation is the particular protein or peptide.

A “fragment” or “segment” is a portion of an amino acid sequence, comprising at least one amino acid, or a portion of a nucleic acid sequence comprising at least one nucleotide. The terms “fragment” and “segment” are used interchangeably herein.

As used herein, a “functional” biological molecule is a biological molecule in a form in which it exhibits a property by which it is characterized. A functional enzyme, for example, is one which exhibits the characteristic catalytic activity by which the enzyme is characterized.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCCS′ and 3′TATGGCS′ share 50% homology.

The determination of “percent identity” between two nucleotide or amino acid sequences can be accomplished using a mathematical algorithm. For example, a mathematical algorithm useful for comparing two sequences is the algorithm of Karlin and Altschul (1990, Proc. Natl. Acad. Sci. USA 87:2264-2268), modified as in Karlin and Altschul (1993, Proc. Natl. Acad. Sci. USA 90:5873-5877). This algorithm is incorporated into the NBLAST and XBLAST programs of Altschul, et al. (1990, J. Mol. Biol. 215:403-410), and can be accessed, for example at the National Center for Biotechnology Information (NCBI) world wide web site having the universal resource locator using the BLAST tool at the NCBI website. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the XBLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al. (1997, Nucleic Acids Res. 25:3389-3402). Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

As used herein, an “instructional material” includes a publication, a recording, a diagram, or any other medium of expression which can be used to communicate the usefulness of the peptide of the invention in the kit for effecting alleviation of the various diseases or disorders recited herein. Optionally, or alternately, the instructional material may describe one or more methods of alleviating the diseases or disorders in a cell or a tissue of a mammal. The instructional material of the kit of the invention may, for example, be affixed to a container which contains the identified compound invention or be shipped together with a container which contains the identified compound. Alternatively, the instructional material may be shipped separately from the container with the intention that the instructional material and the compound be used cooperatively by the recipient.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to a molecule that joins two other molecules either covalently or noncovalently, e.g., through ionic or hydrogen bonds or van der Waals interactions, e.g., a nucleic acid molecule that hybridizes to one complementary sequence at the 5′ end and to another complementary sequence at the 3′ end, thus joining two non-complementary sequences.

As an example of linker technology, see Bausch et al. Clin Cancer Res. 2011 Jan. 15; 17(2): 302-309; in which the Tetramericplectin-1-targeted peptide (tPTP-40 AKTLLPTPGGS(PEG5000))KKKDOTAPA-NH2) SEQ ID NO: 1) was synthesized. In other words, four plectin-targeted peptides (PTPs) were tied to a single DOTA chelator by (4) PEG5000 linkers. The DOTA, of course, then binds a payload, such as therapeutic drug or diagnostic (e.g., radioactive element needed for imaging).

Spacers are also of use in the invention, such as a PEG spacer.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil).

As used herein, the term “nucleic acid” encompasses RNA as well as single and double-stranded DNA and cDNA. Furthermore, the terms, “nucleic acid,” “DNA,” “RNA” and similar terms also include nucleic acid analogs, i.e. analogs having other than a phosphodiester backbone. For example, the so-called “peptide nucleic acids,” which are known in the art and have peptide bonds instead of phosphodiester bonds in the backbone, are considered within the scope of the present invention.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences.”

The term “nucleic acid construct,” as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T.”

The term “otherwise identical sample,” as used herein, refers to a sample similar to a first sample, that is, it is obtained in the same manner from the same subject from the same tissue or fluid, or it refers a similar sample obtained from a different subject. The term “otherwise identical sample from an unaffected subject” refers to a sample obtained from a subject not known to have the disease or disorder being examined. The sample may of course be a standard sample. By analogy, the term “otherwise identical” can also be used regarding regions or tissues in a subject or in an unaffected subject.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

The term “peptide” typically refers to short polypeptides.

As used herein, the term “peptide ligand” (or the word “ligand” in reference to a peptide) refers to a peptide or fragment of a protein that specifically binds to a molecule, such as a protein, carbohydrate, and the like. A receptor or binding partner of the peptide ligand can be essentially any type of molecule such as polypeptide, nucleic acid, carbohydrate, lipid, or any organic derived compound. Specific examples of ligands are peptide ligands of the present inventions.

As used herein, the term “pharmaceutically-acceptable carrier” means a chemical composition with which an appropriate compound or derivative can be combined and which, following the combination, can be used to administer the appropriate compound to a subject. “Pharmaceutically acceptable” means physiologically tolerable, for either human or veterinary application. As used herein, “pharmaceutical compositions” include formulations for human and veterinary use.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross and Mienhofer, eds., The Peptides, vol. 3, pp. 3-88 (Academic Press, New York, 1981) for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl or other acceptable groups linked to the terminal carboxyl group through an ester or ether bond.

As used herein, the term “providing a prognosis” refers to providing information regarding the impact of the presence of the heart disease/disorder (e.g., as determined by the diagnostic methods of the present invention) on a subject's future health.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure. In particular, purified sperm cell DNA refers to DNA that does not produce significant detectable levels of non-sperm cell DNA upon PCR amplification of the purified sperm cell DNA and subsequent analysis of that amplified DNA. A “significant detectable level” is an amount of contaminate that would be visible in the presented data and would need to be addressed/explained during analysis of the forensic evidence.

“Recombinant polynucleotide” refers to a polynucleotide having sequences that are not naturally joined together. An amplified or assembled recombinant polynucleotide may be included in a suitable vector, and the vector can be used to transform a suitable host cell.

As used herein, “modified nucleotides” refers to non-naturally occurring moieties that confer increased thermodynamic stability during hybridization as compared with a polynucleotide that differs from the LNA only by having a natural ribonucleotide in place of the modified RNA nucleotide. In certain embodiments, the ribose moiety of a modified RNA nucleotide is modified with an extra bridge connecting the 2′ oxygen and 4′ carbon. Numerous chemical modifications are commonly used for the synthesis of oligonucleotides for a variety of reasons. For example, to increase the phosphate backbone's stability, adjust duplex stability, change the oligo's conformation, or increase its ability to penetrate a lipid bilayer. Modified sugar moieties are also being incorporated into therapeutic oligonucleotides. Changing the sugar moiety generally increases nuclease resistance and binding affinity to a complementary target.

“Bridged nucleic acid” (“BNA”) refers to 2′-O,4′-C-methylene-modified nucleic acids. In preferred embodiments, BNA, where the 2′ oxygen and 4′ carbon are bridged by a methylene group are used. In other approaches, 2′-0,4′-C-ethylene-bridged nucleic acids (ENA), the 2′ oxygen and 4′ carbon are bridged by an ethylene group. Other examples of BNA can include, but are not limited to, 2′,4′-BNA^{NC}[NH], 2′,4′-BNA^{NC}[NMe], and 2′,4′-BNA^{NC}[NBn].

“Locked nucleic acid nucleotide” (“LNA nucleotide”) as used herein, refers to a modified RNA nucleotide that provides the polynucleotide with greater thermodynamic stability during hybridization as compared with a polynucleotide that differs from the LNA only by having a natural ribonucleotide in place of the modified RNA nucleotide. In certain embodiments, the ribose moiety of a modified RNA nucleotide is modified with an extra bridge connecting the 2′ oxygen and 4′ carbon. LNA nucleotides can comprise any type of extra bridge between the 2′O and 4′C of the RNA that increases the thermodynamic stability of the duplex between the LNA and its complement. In preferred embodiments, BNA, where the 2′ oxygen and 4′ carbon are bridged by a methylene group are used. In other approaches, 2′-0,4′-C-ethylene-bridged nucleic acids (ENA), the 2′ oxygen and 4′ carbon are bridged by an ethylene group. Other examples of BNA can include, but are not limited to, 2′,4′-BNA^{NC}[NH], 2′,4′-BNA^{NC}[NMe], and 2′,4′-BNA^{NC}[NBn].

Other 2′O-modified nucleotides, such as 2′O-Me, demonstrate greater stability, as well.

Oligo backbone configurations that demonstrate particularly high binding affinities to the target (measured by melting temperature or Tm) are preferred for implementing the steric hindrance mechanism. BNA, LNA, FANA, 2′-fluoro, morpholino and piperazine containing backbones are particularly well suited for this purpose. The generation of oligos with mixed linkages such as boranophosphate and phosphate linkages has been accomplished by several solid phase methods including one involving the use of bis(trimethylsiloxy)cyclododecyloxysilyl as the 5′-O-protecting group (Brummel and Caruthers, Tetrahedron Lett 43: 749, 2002). In another example the 5′-hydroxyl is initially protected with a benzhydroxybis(trimethylsilyloxy)silyl group and then deblocked by Et₃N:HF before the next cycle (McCuen et al., J Am Chem Soc 128: 8138, 2006). This method can result in a 99% coupling yield and can be applied to the synthesis of oligos with pure boranophosphate linkages or boranophosphate mixed with phosphodiester, phosphorothioate, phosphorodithioate or methyl phosphonate linkages.

The boranophosphorylating reagent 2-(4-nitrophenyl)ethyl ester of boranophosphoramidate can be used to produce boranophosphate linked oligoribonucleosides This reagent readily reacts with a hydroxyl group on the nucleosides in the presence of 1H-tetrazole as a catalyst. The 2-(4-nitrophenyl)ethyl group can be removed by 1,4-diazabicyclo[5.4.0]undec-7-ene (DBU) through beta-elimination, producing the corresponding nucleoside boranomonophosphates (NMPB) in good yield.

A recombinant polynucleotide may serve a non-coding function (e.g., promoter, origin of replication, ribosome-binding site, etc.) as well.

A host cell that comprises a recombinant polynucleotide is referred to as a “recombinant host cell.” A gene which is expressed in a recombinant host cell wherein the gene comprises a recombinant polynucleotide, produces a “recombinant polypeptide.”

A “recombinant polypeptide” is one which is produced upon expression of a recombinant polynucleotide.

A “recombinant cell” is a cell that comprises a transgene. Such a cell may be a eukaryotic or a prokaryotic cell. Also, the transgenic cell encompasses, but is not limited to, an embryonic stem cell comprising the transgene, a cell obtained from a chimeric mammal derived from a transgenic embryonic stem cell where the cell comprises the transgene, a cell obtained from a transgenic mammal, or fetal or placental tissue thereof, and a prokaryotic cell comprising the transgene.

The term “modulate” refers to either stimulating or inhibiting a function or activity of interest.

By the term “specifically binds to,” as used herein, is meant when a compound or ligand functions in a binding reaction or assay conditions which is determinative of the presence of the compound in a sample of heterogeneous compounds, or it means that one molecule, such as a binding moiety, e.g., an oligonucleotide or antibody, binds to another molecule, such as a target molecule, e.g., a nucleic acid or a protein, in the presence of other molecules in a sample.

The terms “specific binding” or “specifically binding” when used in reference to the interaction of a peptide (ligand) and a receptor (molecule) also refers to an interaction that is dependent upon the presence of a particular structure (i.e., an amino sequence of a ligand or a ligand binding domain within a protein); in other words the peptide comprises a structure allowing recognition and binding to a specific protein structure within a binding partner rather than to molecules in general. For example, if a ligand is specific for binding pocket “A,” in a reaction containing labeled peptide ligand “A” (such as an isolated phage displayed peptide or isolated synthetic peptide) and unlabeled “A” in the presence of a protein comprising a binding pocket A the unlabeled peptide ligand will reduce the amount of labeled peptide ligand bound to the binding partner, in other words a competitive binding assay.

The term “standard,” as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. Standard can also refer to an “internal standard”, such as an agent or compound which is added at known amounts to a sample and is useful in determining such things as purification or recovery rates when a sample is processed or subjected to purification or extraction procedures before a marker of interest is measured. Internal standards are often a purified marker of interest which has been labeled, such as with a radioactive isotope, allowing it to be distinguished from an endogenous marker.

As used herein, a “subject in need thereof” is a patient, animal, mammal, or human, who will benefit from the method of this invention.

As used herein, a “substantially homologous amino acid sequences” includes those amino acid sequences which have at least about 95% homology, at least about 96% homology, at least about 97% homology, at least about 98% homology, or at least about 99% or more homology to an amino acid sequence of a reference antibody chain Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the present invention.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. Preferably, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm.

The term “substantially pure” describes a compound, e.g., a protein or polypeptide which has been separated from components which naturally accompany it. Typically, a compound is substantially pure when at least 10%, at least 20%, at least 50%, at least 60%, at least 75%, at least 90%, or at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

The term “symptom,” as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse and other observers.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

Methods involving conventional molecular biology techniques are described herein. Such techniques are generally known in the art and are described in detail in methodology treatises, such as Molecular Cloning: A Laboratory Manual, 2nd ed., vol. 1-3, ed. Sambrook et al., Cold Spring Harbor Laboratory Press, Cold Spring Harbor, N.Y., 1989; and Current Protocols in Molecular Biology, ed. Ausubel et al., Greene Publishing and Wiley-Interscience, New York, 1992 (with periodic updates). Methods for chemical synthesis of nucleic acids are discussed, for example, in Beaucage and Carruthers, Tetra. Letts. 22: 1859-1862, 1981, and Matteucci et al., J. Am. Chem. Soc. 103:3185, 1981.

### Kits

In certain embodiments, the nanoparticle comprising compositions of the invention and instructions for use thereof are provided in a kit for use in research and for treating diseases (such as cancer), as well as unit doses for uses described herein.

The compositions, formulations, unit dosages, and articles of manufacture described herein are for use in the methods of treatment, methods of administration, and dosage regimes described herein. Kits of the invention include one or more containers comprising the p52K-containing nanoparticle compositions (formulations or unit dosage forms and/or articles of manufacture), and in some embodiments, further comprise instructions for use in accordance with any of the methods of use or treatment described herein. In some embodiments, the kit comprises i) a composition comprising nanoparticles comprising a recombinant nucleic acid, synthetic peptide, drug or hydrophobic drug derivative and optionally a carrier protein (such as albumin) and ii) instructions for administering the nanoparticles and the therapeutic agents simultaneously and/or sequentially, for treatment of disease.

Instructions supplied in the kits of the invention are typically written instructions on a label or package insert (e.g., a paper sheet included in the kit), but machine-readable instructions (e.g., instructions carried on a magnetic or optical storage disk) are also acceptable. The instructions relating to the use of the nanoparticle compositions generally include information as to dosage, dosing schedule, and route of administration for the intended treatment. The kit may further comprise a description of selecting an individual suitable or treatment.

The present invention also provides kits comprising compositions (or unit dosages forms and/or articles of manufacture) described herein and may further comprise instruction(s) on methods of using the composition, such as uses further described herein. In some embodiments, the kit of the invention comprises packaging. In other variations, the kit of the invention comprises a plurality of different types of packaging, e.g., a second packaging comprising a buffer. It may further include other materials desirable from a commercial and user standpoint, including other buffers, diluents, filters, needles, syringes, and package inserts with instructions for performing any methods described herein.

For combination therapies of the invention, the kit may contain instructions for administering the first and second therapies simultaneously and/or sequentially for the effective treatment of disease. The first and second therapies can be present in separate containers or in a single container. It is understood that the kit may comprise one distinct composition or two or more compositions wherein one composition comprises a first therapy and one composition comprises a second therapy.

The materials and methods below are provided to facilitate practice of the claimed invention.

### Cell Culture.

All cell lines were obtained from the American Type Culture Collection (ATCC) and cultured under standard conditions at 37° C. and 5% C02. Primary like human bronchial epithelial cells HBE C3-KT (Cat #: ATCC CRL-4051) were grown in Airway Epithelial Cell Basal Medium (Cat #: ATCC PCS-300-030) supplemented with Bronchial Epithelial Cell Growth Kit (Cat #: ATCC PCS-300-040) and 1% Pen/Strep. 293 (Cat #: ATCC CRL-1573) and 293T (Cat #: ATCC CRL-3216) cells were grown in DMEM (Corning, Cat #: 10-013-CV) supplemented with 10% FBS (VWR, Cat #: 89510-186) and 1% Pen/Strep (Gibco, Cat #: 15140-122). A549 cells (Cat #: ATCC CCL-185) were maintained in Ham's F-12K medium (Gibco, Cat #: 21127-022) supplemented with 10% FBS and 1% Pen/Strep. All cell lines tested negative for mycoplasma using the LookOut Mycoplasma PCR Detection Kit (Sigma-Aldrich).

### Transgenic Cell Lines.

The inducible 52K, Δ1-47, 52K-GFP, or GFP expressing stable transgenic cell lines (A549:52K, A549:Δ1-47, A549:52K-GFP, A549:GFP) were generated by lentivirus transduction and selection of A549 lung adenocarcinoma cells. Lentivirus was generated by co-transfection of 293T cells with lentivirus expression plasmid in addition to helper plasmids pMD.G2 and pCMVAR8.74 (see plasmids and transfections). Cell supernatant was collected at 48 and 72 hours and filtered through 0.45 μM Acrodisc syringe filters (VWR, Cat #: 28143-312) using a syringe. Cells were transduced by incubation with lentivirus supernatant in the presence of 10 μg/mL polybrene (Santa Cruz, Cat #: sc-134220) for 16 h, and lentivirus supernatant replaced with fresh culture media. Cells were cultured for a further 24 h before selection puromycin (Gibco, cat #: A1113802) at a final concentration of (1 μg/ml).

### Adenoviruses and Infection.

Ad5 wild-type (WT) was purchased from ATCC and propagated on 293 cells. The Ad5 Δ52K mutant pm8001, a gift from P. Hearing (Stony Brook University, NY), was propagated on transgenic cell line A549:52K. Viruses were purified using two sequential rounds of ultracentrifugation in CsCl gradient and stored in 40% glycerol at −20° C. Ad5 WT titers were determined by plaque assay on HEK293 cells. Pm8001 titers were determined by a TCID50 based approach using immunofluorescent visualization of the viral DNA binding protein as an indicator of infected cells. All infections were carried out by standard protocols using a multiplicity of infection (MOI) of 10 and harvested at indicated hours post infection (hpi). To reduce heterogeneity in the progress of infection, HBEC3-KT cells were grown to confluency before infection. To infect cells, virus was diluted in DMEM containing 2% FBS and 1% Pen/Strep (for A549 cells) or Airway Epithelial Cell Basal Medium supplemented with Bronchial Epithelial Cell Growth Kit and 1% Pen/Strep (for HBEC3-KT). After 2 h at 37° C. additional DMEM containing 10% FBS (Airway Epithelial Cell Basal Medium supplemented with Bronchial Epithelial Cell Growth Kit and 1% Pen/Strep for HBEC3-KT) was added. For virus yield assays, the virus infection media was removed after 2 h and cells were washed 1× in PBS before addition of DMEM or Airway Epithelial Cell Basal Medium to remove excess virus.

### Plasmids and Transfection.

To generate mammalian expression plasmids, open reading frames were PCR amplified and inserted into parent plasmids by restriction digest and ligation. For fusion proteins tagged with GFP, open reading frames were inserted into pEGFP-N1 (Clontech) containing a L221K mutation to prevent dimerization of GFP molecules¹. Untagged 52K or Δ1-47 open reading frames were inserted into pEYFP-C1 (Clontech) in place of the EYFP open reading frame. For expression of MBP-tagged fusion proteins in bacteria, open reading frames were PCR amplified and inserted into pMAL-c2X (Addgene, plasmid #: 75286) using a fragment assembly strategy and Gibson Assembly Master Mix (NEB, Cat #: E2611S) following the manufacturers' instructions. pLKO.dCMV.TetO/R was a gift from Chris Boutell². Lentivirus expression plasmids pLKO.dCMV.TetO/R.52K and pLKO.dCMV.TetO/R.Δ1-47 were generated by inserting excised open reading frames into pLKO.dCMV.TetO/R in the place of the EYFP open reading frame using the NheI and SalI restriction sites. To generate lentivirus expression plasmids pLKOTetO/R.mGFP and pLKOTetO/R.52K-mGFP, mGFP or 52K-mGFP open reading frames were PCR amplified and cloned using the above strategy. Lentivirus plasmids pMD.G2 (plasmid #12259) and pCMVAR8.74 (plasmid #22036) were purchased from Addgene. All plasmids generated as part of this study were checked by restriction digest and Sanger sequencing to confirm correct insertion of the open reading frame.

### Transgene Expression in Mammalian Cells.

For transient expression of transgenes, mammalian expression plasmids were transfected into 293T cells using X-tremeGENE HP (Roche, Cat #: 6366236001), following the manufacturer's instructions. Transfected cells were analyzed 48 h post-transfection, or infected 24 h post-transfection and analyzed 24 h post-infection. For stable cell lines, transgene expression was induced by addition of doxycycline at a final concentration of (1 μg/ml) and analyzed 24 h post-induction. For transgene expression during infection, expression was induced 2 h post-infection.

### Antibodies.

The following primary antibodies for viral proteins were used: Adenovirus late protein antibody recognizing Hexon, Penton and Fiber (gift from J. Wilson), species: rabbit, polyclonal, WB 1:10,000. 52K (gift from P. Hearing), species: rabbit, polyclonal, WB 1:10000, IF 1:500. IIIa (gift from P. Hearing), species: rabbit, polyclonal, WB 1:5000, IF 1:500. 100K (gift from P. Hearing), species: rabbit, polyclonal, WB 1:10000. 33K (gift from P. Hearing), species: rabbit, polyclonal, IF 1:500. DBP (gift from A. Levine), species: mouse, clone: B6-8, WB 1:1000, IF 1:400. EIB55K (gift from A. Levine), species: mouse, clone: 58K2AG, WB 1:500, IF 1:250. E4orf6 (gift from D. Ornelles), species: mouse, clone: RSA #3, WB 1:500, IF 1:250. Hexon trimer (Developmental Studies Hybridoma bank, Cat #: TC31-9C12.C9), species: mouse, clone: 9C12, IF 1:250. Fiber trimer (Genetex, Cat #: GTX23232), species: mouse, clone: 2A6, IF 1:250. The following primary antibodies for cellular proteins were used: GFP (Abcam, Cat #: ab290), species: rabbit, polyclonal, WB 1:5000. Actin (Sigma-Aldrich, Cat #: A5441), species: mouse, clone: AC-15, Lot: 064M4789V, WB 1:5000. Tubulin (Santa Cruz Biotechnology, Cat #: sc-69969), species: mouse, clone: 6A204, Lot: D0412, WB 1:1000. GAPDH (GeneTex, Cat #: GTX100118), species: rabbit, polyclonal, WB 1;10000. Histone H3 (Abcam, Cat #: ab1791), species: rabbit, polyclonal, Lot: GR3198176-1, WB 1:5,000. Lamin A (Santa-Cruz, Cat #: sc-7293), species: mouse, clone: 346, IF 1:500. Histone H1 (Abcam, Cat #: ab4269), species: mouse, clone AE4, IF 1:500).

For western blotting, Horseradish peroxidase-conjugated (HRP) goat anti-mouse (Jackson Laboratories, Cat #: 115-035-003) or goat anti-rabbit (Jackson Laboratories, Cat #: 111-035-045) secondary antibodies were used at a concentration of 1:10000-1:50000. For IF, the following Alexa Fluor fluorophore-conjugated secondaries were used at a concentration of 1:1000: goat anti-rabbit 488 (Life Technologies, Cat #: A-11008), goat anti-mouse 488 (Life Technologies, Cat #: A-11001), goat anti-mouse 555 (Life Technologies, Cat #: A-21422), and goat anti-rabbit 647 (Life Technologies, Cat #: A-21245).

### Visualization of Newly Synthesized Proteins.

Cells were first incubated with methionine free culture media for 10 mins to deplete cells of methionine. Cells were then incubated with 1 mM L-Homopropargylglycine (HPG) (Thermo Fischer Scientific, Cat #: C10186) or DMSO control for 30 minutes prior to fixation and subsequent permeabilization (see immunofluorescence). Incorporated HPG was conjugated to a fluorophore using a copper catalyzed cycloaddition (click chemistry) reaction as follows. Cells were incubated with freshly made click buffer containing 5 μM 488 picolyl-azide (Click Chemistry Tools, Cat #: 1276-1), 1 mM μM copper sulfate (Sigma-Aldrich, Cat #: C7631), and 2.5 μM Sodium Ascorbate (Sigma-Aldrich, Cat #: A4034) in PBS for 2 mins. Cycloaddition buffer was removed, and cells washed 3 times with PBS before additional staining. Newly synthesized protein was visualized as for immunofluorescence.

### Immunofluorescence Confocal Microscopy.

Cells were grown on 12 mm glass coverslips (Electron Microscopy Sciences, Cat #: 72196-12) in 24-well plates. Cells were fixed in 4% PFA in PBS for 10 mins, followed by permeabilization with 0.5% Triton-X in PBS for 10 mins. The samples were blocked in 3% BSA in PBS (+0.05% sodium azide) for 30 mins, incubated with primary antibodies in 3% BSA in PBS (+0.05% sodium azide) for 1 h, followed by secondary antibodies and 4,6-diamidino-2-phenylindole (DAPI) for 1 h. Coverslips were mounted onto glass slides using ProLong Gold Antifade Reagent (Cell Signaling Technologies, cat: #: 9071). Immunofluorescence was visualized using a Zeiss LSM 710 Confocal microscope (Cell and Developmental Microscopy Core at UPenn) and ZEN 2011 software. Images were processed in ImageJ using equivalent settings.

### Image Analysis.

Image analysis was carried out using ImageJ. For analysis of size or fluorescence intensity, nuclei or in vitro phase separated droplets were selected as regions of interest (ROI) and analyzed using the analyze particles and measure functions. ROIs were selected using the gaussian blur (sigma radius 1), fluorescence intensity thresholding, and binary object selection functions. Nuclei were determined to be antigen positive if their mean nuclear fluorescence intensity was greater than the mean+2 standard deviations of mean nuclear fluorescence intensity in negative control (uninfected or mock transfected) nuclei. Line profile analysis was carried out using image J using the plot profile function, and fluorescence determined as relative intensity compared to the maximum value measured (set to 1). Phenotype analysis was performed blinded, numerating the number of nuclei corresponding to pre-defined phenotype categories. For all image analysis, a minimum of 3 fields of view were analyzed for each replicate. For localization phenotype analysis of ectopically expressed GFP-tagged fusion proteins, or endogenous IIIa, cells negative for expression of the corresponding proteins were excluded from the analysis.

Live Cell Imaging and Fluorescence Recovery after Photo-Bleaching (FRAP).

Live cell imaging was performed on a spinning disk confocal microscope. Photobleaching was performed using the 488 nm laser line from an Argon ion laser, with fixed bleach spot diameter of 2.25 μm. Fluorescence recovery was monitored for a minimum of 20 seconds. Fluorescence recovery was analyzed using ImageJ as previously described³. For assessment of fusion events, 5 fields of view were imaged for 5 min. Cells exhibiting punctate VLNBs were observed, and fusion events numerated.

### Coomassie and Western Blotting.

Western blot analysis was carried out using standard methods. In brief, protein samples were prepared using lithium dodecyl sulfate (LDS) loading buffer (NuPage) supplemented with 25 mM Dithiothreitol (DTT) and boiled at 95° C. for 10 min. Equal amounts of protein lysate were separated by SDS-PAGE. For Coomassie, gels were stained with 0.1% w/v brilliant blue (Sigma-Aldrich, Cat #: B0149) in 10% v/v acetic acid and 40% v/v methanol for 10 min before de-staining in 10% v/v acetic acid and 40% v/v methanol. For immunoblotting, proteins were transferred onto a nitrocellulose membrane (Millipore) at 30 V for at least 60-120 min. Membranes were stained with Ponceau to confirm equal loading and transfer, and then blocked in blocking buffer (5% milk in TBST supplemented with 0.05% sodium azide) for 1 h. Membranes were incubated overnight with primary antibodies diluted in blocking buffer, washed for 30 min in TBST, incubated for 1 h with HRP-conjugated secondary antibody diluted in TBST and washed again for 30 min in TBST. Proteins were visualized with Pierce ECL Western Blotting Substrate (Thermo Scientific) and detected using a Syngene G-Box. Images were processed and assembled in Adobe CS6.

Viral Genome Accumulation by qPCR.

Infected cells were harvested by Trypsin digestion at the indicated times post-infection and total DNA was isolated using the PureLink Genomic DNA kit (Invitrogen, Cat #: K182002) following the manufacturer's instructions. qPCR was performed using primers for the Ad5 DBP and cellular tubulin as previously described⁴. Values for DBP were normalized internally to tubulin and to the 4 hpi time point to control for any variations in virus input. qPCR was performed on the QuantStudio 7 Flex Real-Time PCR System using SYBR Green PCR Master Mix (Applied Biosystems, Cat #: 4309155) following the manufacturer's instructions. Relative genome copy number was analyzed using the ΔΔCT method using tubulin as the internal control. For increased accuracy, qPCR of each biological replicate was performed in technical triplicate.

### Viral Progeny Production.

Infected cells were harvested by scraping and lysed by three cycles of freeze-thawing. Cells were harvested at 48 hpi unless stated otherwise. Cell debris was removed from lysates by centrifugation at max speed, 4° C., 5 min. For analysis of WT AdV infectious progeny production, lysates were diluted serially in DMEM supplemented with 2% FBS and 1% Pen/Strep and used to infect HEK293 cells. Infection media was removed 2 h after infection, and cells were overlaid with DMEM containing 0.45% SeaPlaque agarose (Lonza, cat #: 50101) in addition to 2% FBS and 1% Pen/Strep. Overlaid cells were incubated until visible plaques in the cell monolayer appeared (5-6 days post-infection). Plaques were stained using 1% w/v crystal violet in 50% v/v Ethanol. Plaques were counted and the number of plaque forming units (PFU) calculated. For analysis of Δ52K mutant virus pm8001, lysates were diluted serially in DMEM supplemented with 2% FBS and 1% Pen/Strep and used to infect A549 cells. Infection media was removed 2 h after infection, and cells were overlaid with growth media. Cells were incubated for 24 h prior to fixation and analysis by immunofluorescence confocal microscopy using immunostaining of the viral DNA binding protein (DBP) as an indicator of infection. For each experimental repeat, 3 fields of view were captured and the percentage of DBP positive cells determined. The serial dilution resulting in closest to 50% DBP-positive cells was selected for calculation of progeny production. Total cell number was determined by counting cells grown in parallel under equivalent conditions. The number of focus forming units (FFU) was calculated as the product of total cell number and % of antigen positive cells, adjusting for the Poisson distribution. For analysis of packaged or unpackaged viral particles, large scale lysates were generated from 10 15 cm plates of confluent cells (approx. 600 million cells). Cells were harvested by trypsinization, pooled, and pelleted by centrifugation. Cell pellets were lysed by three cycles of freeze-thawing, and cell debris removed by centrifugation at max speed, 4° C., 5 min. PBS was added to lysates to a final volume of 7 ml. To prepare cesium chloride (CsCl) density gradients, 2.5 ml 1.34 g/cc CsCl was added to 13.2 mL Ultra-Clear ultracentrifuge tubes (Beckman-Coulter) and carefully underlaid with 2.5 ml 1.43 g/cc CsCl. Clarified viral lysates were gently layered on top. Loaded samples were centrifuged at 25,000 rpm for 2 h at 4° C. using an Optima XPN-80 ultracentrifuge (Beckman-Coulter), and SW-41 Ti rotor. Tubes were removed from the ultracentrifuge and placed in a suspended laboratory clamp for imaging and harvesting of AdV particle bands. Both unpackaged and packaged bands were harvested in a sterile 15 mL conical tube, resuspended in 7 mL of sterile PBS, and layered on top of a fresh CsCl density gradient. Tubes were spun a second time in the ultracentrifuge at 25,000 rpm for 16 h at 4° C. to achieve optimal separation of packaged and unpackaged particle bands. Tubes were then removed from the ultracentrifuge and placed in a tube rack for final imaging.

1,6-hexanediol.

For acute disruption of viral late nuclear bodies, 1,6-hexanediol (Sigma-Aldrich, Cat #: 240117) was diluted in cell culture media at 10% w/v. Cell culture media was replaced with media containing 10% 1,6-hexanediol or fresh culture media and incubated for 10 mins at 37° C. prior to fixation. For long-term incubation, cell culture media was replaced with fresh cell culture media, or media containing 100 mM (1.18%). To assess the impact on viral replication compartment formation, cells were incubated with 100 mM 1,6-hexanediol from 2 hours post-infection onwards. For other experiments, infected cells were incubated with 100 mM 1,6-hexanediol from 12 hours post-infection onwards unless stated otherwise.

### Cell Viability.

Assessment of cell membrane integrity (live/dead assay) was carried out using 0.4% trypan blue (Invitrogen, Cat #: T10282), cell counting chamber slides (Invitrogen, Cat #: C10228) and a Countess Automated Cell Counter (Thermo Fischer Scientific).

### Purification of Recombinant Proteins.

For expression of MBP fusion proteins, B121 De3 LysS cells (NEB, Cat #: C2527H) were transformed with bacterial expression plasmids by heat shock at 42° C. and selected on LB containing chloramphenicol, carbenicillin, and 0.5% w/v glucose for 16 h at 37° C. Colonies were used to inoculate LB broth containing chloramphenicol, carbenicillin, and 0.5% glucose and cultured at 37° C. in a shaking incubator until an optical density (600 nm) of 0.4-0.6 was reached. Expression was induced by the addition of 0.5 mM IPTG (Roche, Cat #: 10724815001) for 16 h at 15° C. Cultures were pelleted by centrifugation and frozen prior to purification. Pellets were thawed/resuspended in lysis/binding buffer supplemented with Halt Protease Inhibitor cocktail (Thermo Fischer Scientific, Cat #: 78445). The lysates were incubated with Benzonase (Sigma-Aldrich, Cat #: E1014) at a final concentration of 250 Units/mL for 1 h at 4° C. before sonication (×6 cycles of 20 seconds on-20 seconds off on “low” setting in 4° C. water bath), and the lysates clarified by centrifugation. Protein was purified by affinity purification using Express Ni resin (NEB, Cat #: S1428S) following the manufacturers guidelines with the following modifications. Express Ni resin was pre-equilibrated with Ni lysis/binding buffer (20 mM sodium phosphate, 500 mM NaCl, 1 mM DTT, pH 8), washed in Ni wash buffer (20 mM sodium phosphate, 300 mM NaCl, 2.5% glycerol, 5 mM Imidazole, 1 mM DTT, pH 8), and eluted in Ni elution buffer (20 mM sodium phosphate, 300 mM NaCl, 2.5% glycerol, 500 mM Imidazole, 1 mM DTT, pH 8). Purification was performed at 4° C. to limit protein degradation. Purity of protein was confirmed to be >90% as assessed by SDS-PAGE and Coomassie staining. All purified protein or labelled purified proteins were sub-aliquoted and stored at −80° C. to limit protein degradation. Protein concentration was determined by Bradford assay using Pierce Bradford reagent (Thermo Fischer Scientific, Cat #: 23246) and NanoDrop 2000c spectrophotometer (Thermo Fischer Scientific).

### Buffer Exchange and Concentration of Purified Proteins.

Purified proteins were buffer exchanged and concentrated using Amicon 10 kDa spin filters (EMD Millipore, Cat #: UFC801008) using the manufacturers guidelines. For buffer exchange, protein samples were concentrated a minimum of 20-fold prior to re-dilution in the exchange buffer. Concentration and re-dilution was repeated a total of 3 times to achieve a dilution factor in excess of 8000.

### Fluorescent Labelling of Purified Protein.

Purified protein was buffer exchanged and concentrated in 0.1 M sodium bicarbonate buffer, pH 8. 0.5 mg of AZDye 488 NHS Ester (Click Chemistry Tools, Cat #: 1338-1) was dissolved in 100 μL of DMSO immediately before addition to 10 mg of protein at a concentration of 10 mg/mL. The reaction was incubated at 25° C. for 1 h whilst nutating. The reaction was quenched by addition of 100 μL freshly prepared 1.5 M hydroxylamine, pH 8.5. Labelled protein was separated from unreacted dye by buffer exchange into phase-separation buffer (see In vitro phase-separation assays).

Purification of Viral Genomic DNA (vDNA).

Double stranded DNA corresponding to the full-length genome of AdV serotype 5 was excised from plasmid pTG3602 by restriction digest with PacI and separated by agarose gel-electrophoresis. DNA was purified using the QIAEXII Gel Extraction Kit (Qiagen, Cat #: 20021) using the manufacturer's instructions, eluting in 25 mM Tris-HCl, pH 7.4. Extracted DNA was exchanged into phase-separation buffer and concentrated using Amicon 10k spin-filters as described for purified protein. Purified protein was checked by agarose gel-electrophoresis in 1% agarose gel using GelRed for detection. For generation of benzonase-digested DNA stocks, 250 Units of benzonase were added to 1 μg of purified DNA and incubated for 4 h at 25° C.

### In Vitro Phase-Separation Assays.

In vitro phase-separation assays were carried out in separation buffer (25 mM Tris-HCl, 150 mM NaCl, 1 mM DTT, pH 7.4) using the indicated concentration of proteins and DNA in a final volume of 25 μL. TEV protease (A kind gift from Rina Fujiwara) was added at w/w ratio of 1:50, and the reaction incubated at 25° C. for 30 minutes prior to transfer to a 384 well plate. Condensates were allowed to settle for 5 minutes prior to imaging using an EVOS imaging system (Thermo Fischer Scientific). Unless indicated otherwise, assays were imaged within a 15-minute window to limit variability caused by condensate ripening. Protein samples were centrifuged to remove any debris, and concentration checked prior to use. Turbidity of samples was analyzed by O.D₆₀₀ using NanoDrop One spectrophotometer (Thermo Fischer Scientific) as per the manufacturer's guidelines.

### Size Exclusion Chromatography of Nanoparticles

Size exclusion column was prepared using 40 ml of resuspended Sephacryl S500 in a 2 cm diameter, 30 cm length Konte column. Excess liquid was allowed to flow through the column until the meniscus sat above the resin bed. The column equilibrated with LLPS buffer (25 mM Tris-HCl, 150 mM NaCl, 1 mM DTT, pH 7.4) by 4 sequential rounds of buffer additional and flow through, adding 15 ml of LLPS buffer each time. 200 l of in vitro assembly reaction was added directly above the resin bed. The column flow was activated to draw the sample into the resin and then immediately halted. The column and column reservoir were carefully filled with LLPS buffer. The column flow was activated, and sequential 1 ml fractions (1-26) were collected. These samples were analyzed by SDS-PAGE and western blot, or quantitative PCR.

Detection of vDNA by Quantitative PCR (qPCR).

Phenol-chloroform extraction was used to purify DNA from fractions isolated by size exclusion chromatography. The extracted DNA containing phase was analyzed for the presence of vDNA by qPCR, using primers specific for the viral DNA binding protein open reading frame present in the viral genome. qPCR was performed on the Applied Biosystems Viia 7 Real-Time PCR System operating on QuantStudio 7 software using SYBR Green PCR Master Mix (Applied Biosystems, Cat #: 4309155) and following the manufacturer's instructions for standard speed reactions. For increased accuracy, ChIP-qPCRs were performed in technical triplicate.

Amplification and Purification of Circular Plasmid DNA (pTG3602).

Chemically competent E. coli (strain DH5a) were transformed with plasmid pTG3602 by heat shock at 42° C. for 45 seconds, incubated on ice for 2 minutes. The bacteria were recovered by addition of S.O.C broth and incubation in a shaking incubator for 1 hour at 37° C. Recovered bacteria were plated on LB agar plates containing 100 g/ml ampicillin and incubated for 16 hours at 37° C. A single clonal colonies was selected and used to inoculate a 5 ml starter culture of LB broth containing 100 g/ml ampicillin. The starter culture was incubated in a shaking incubator for 8 hours at 37° C. After incubation, 2 ml of the starter culture were added to a 250 ml primary culture of LB broth containing 100 g/ml ampicillin. The primary culture was incubated in a shaking incubator for 16 hours at 37° C. After incubation, the primary culture was pelleted by centrifugation at 4000 G for 20 minutes. The pellet was resuspended, lysed, and plasmid DNA purified using a PureLink™ HiPure Plasmid Maxiprep Kit (Thermo Scientific #K210006) following the manufacturer's instructions. For incorporation into nanoparticles, purified plasmid DNA was first diluted in phase-separation buffer (25 mM Tris-HCl, 150 mM NaCl, 1 mM DTT, pH 7.4).

### Bioinformatics.

The adenovirus serotype 5 52K protein (aka L1 packaging protein 3) amino acid sequence was obtained from Uniprot (Q6VGV2). Also see Uniprot P04496. Disorder tendency was obtained using the IUPred2A algorithm (https://iupred2a.elte.hu/) using the default settings⁵. Fold index was obtained using the PLAAC algorithm (http://plaac.wi.mit.edu)⁶. The “core length” was set as 60 (algorithm default). The “relative weighting of background probabilities a” was set as 0 and was calculated “from input sequence”. The fraction of charged residues (FCR), net charge per residue (NCPR), and hydrophobicity were calculated from the Classification of Intrinsically Disordered Ensemble Regions (CIDER) algorithm (http://pappulab.wustl.edu/CIDER/)⁷. CIDER was run locally using localCIDER with the sequence Parameters module. The FCR, NCPR, and hydrophobicity were obtained using the commands “get_linear_FCR”, “get_linear_NCPR”, and “get_linear_hydropathy”, respectively. All localCIDER commands were executed using default parameters. The PiPi score was calculated using the Phase Separation Predictor algorithm to predict propensity for pi-pi contacts (http://abragam.med.utoronto.ca/˜JFKlab/Software/psp.htm) using the default parameters⁸. The protein amino acid frequencies were calculated for the specified regions of Ad5 52-55K (full length=“FL”, residues 1-47, residues 1-119, residues 48-119, and residues 48-415). The 52-55K amino acid frequencies were compared to background frequencies calculated from all human proteins obtained by a search of all “human” organism Uniprot/SwissProt entries (20,371 protein sequences). Additionally, the 52-55K amino acid frequencies were compared to the amino acid frequencies calculated from a curated set of phase separating proteins (PPS proteins)⁹. For the human protein sequences and the PPS protein sequences, the amino acid frequencies were calculated on a per-sequence basis and the frequencies were averaged over each protein in the respective set to calculate the average and standard deviation.

### Reproducibility and Statistics.

All experimental observations were confirmed by fully independent repeat experiments. Where data from independent repeats are pooled, the experimental outcome was confirmed to be consistent between repeats. Unless stated otherwise, the mean of numerical data is shown, with error bars representing standard deviation. Statistics were performed using GraphPad Prism version 9. All t-tests are two-sided. Full details of sample size and statistical tests used including test statistics, exact P values, assumptions, and adjustments for multiple corrections are included with source data.

The references below are provided to facilitate practice of the methods disclosed and are incorporated by reference herein.
1. Zacharias, D. A., Violin, J. D., Newton, A. C. & Tsien, R. Y. Partitioning of lipid-modified monomeric GFPs into membrane microdomains of live cells. Science 296, 913-916 (2002).
2. Busnadiego, I. et al. Host and Viral Determinants of Mx2 Antiretroviral Activity. J Virol 88, 7738-7752 (2014).
3. Blumenthal, D., Goldstien, L., Edidin, M. & Gheber, L. A. Universal Approach to FRAP Analysis of Arbitrary Bleaching Patterns. Sci Rep 5, (2015).
4. Price, A. M. et al. Direct RNA sequencing reveals m6A modifications on adenovirus RNA are necessary for efficient splicing. Nat Commun 11, 6016 (2020).
5. Mészáros, B., Erdos, G. & Dosztinyi, Z. IUPred2A: context-dependent prediction of protein disorder as a function of redox state and protein binding. Nucleic Acids Res 46, W329-W337 (2018).
6. Lancaster, A. K., Nutter-Upham, A., Lindquist, S. & King, O. D. PLAAC: a web and command-line application to identify proteins with prion-like amino acid composition. Bioinformatics 30, 2501-2502 (2014).
7. Holehouse, A. S., Das, R. K., Ahad, J. N., Richardson, M. O. G. & Pappu, R. V. CIDER: Resources to Analyze Sequence-Ensemble Relationships of Intrinsically Disordered Proteins. Biophys J 112, 16-21 (2017).
8. Vernon, R. M. et al. Pi-Pi contacts are an overlooked protein feature relevant to phase separation. eLife 7, e31486.
9. van Mierlo, G. et al. Predicting protein condensate formation using machine learning. Cell Rep 34, 108705 (2021).

The following examples are provided to illustrate certain embodiments of the invention. They are not intended to limit the invention in any way.

### Example 1

### Biomolecular Condensates and Assembly of Viral Particles

To investigate the role of BMCs during infection, we capitalized on the tractability of AdV, a prototype nuclear-replicating DNA virus that induces significant remodeling of the host-cell nucleus¹². Unlike many RNA viruses, the AdV proteins involved in DNA replication are distinct from those involved in particle assembly and genome packaging^{13,14} We reasoned that this would allow for genetic approaches to dissect the role of BMCs in specific viral processes. By interrogating the spatial organization of progeny assembly, and the phase-separation and function of a dedicated packaging protein, we demonstrate an essential mechanistic role for phase-separation in the assembly of infectious progeny particles.

Viral Structural Proteins Accumulate in Nuclear Bodies Distinct from Viral Replication Compartments.

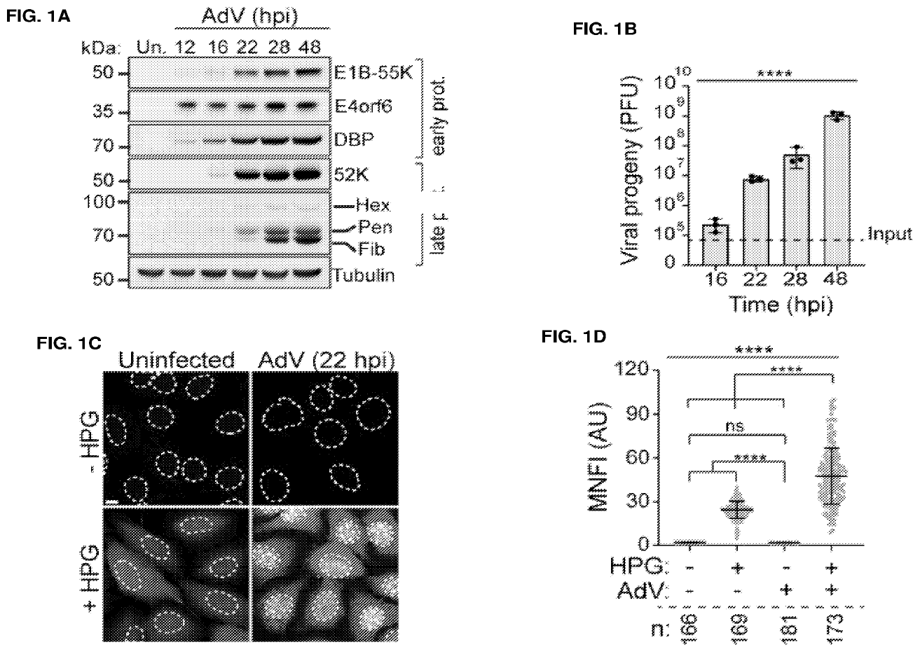

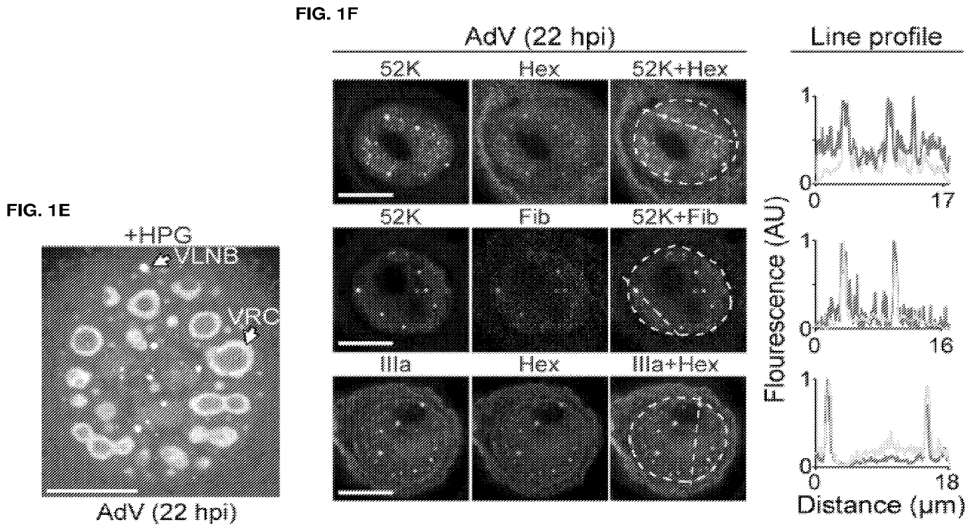

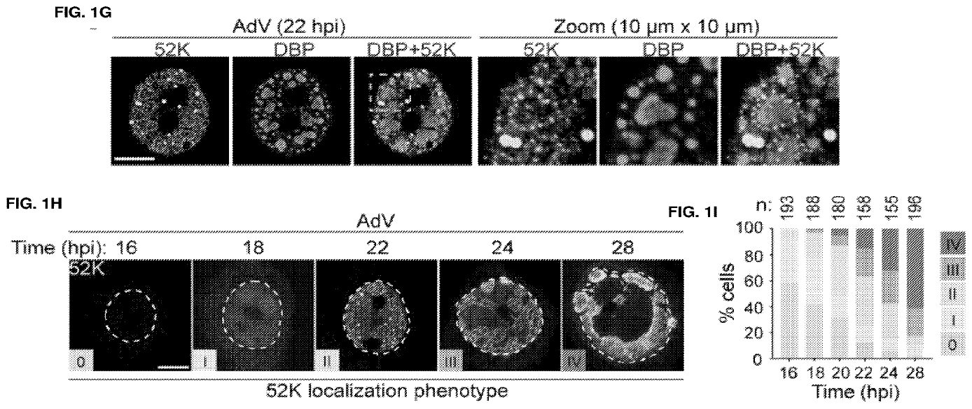

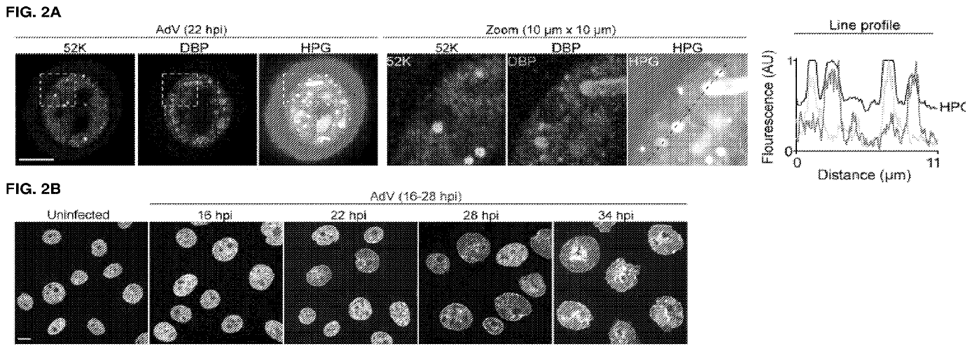

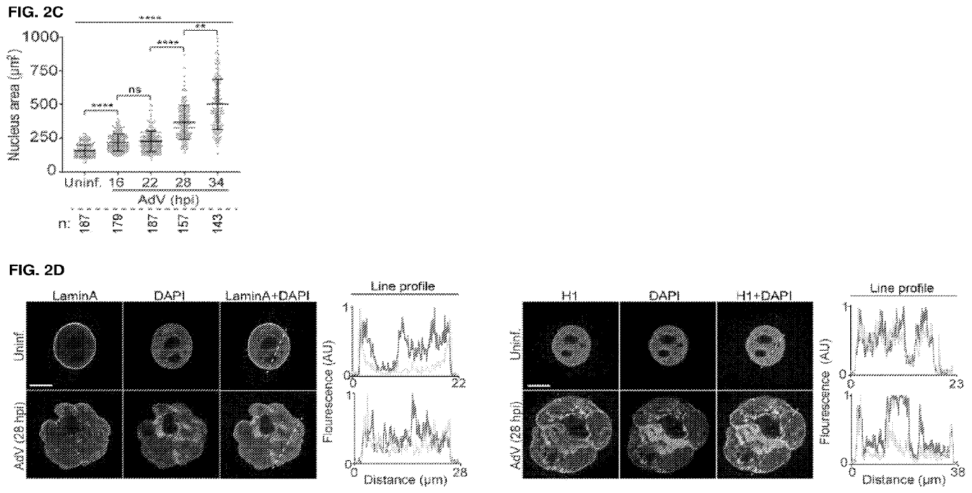

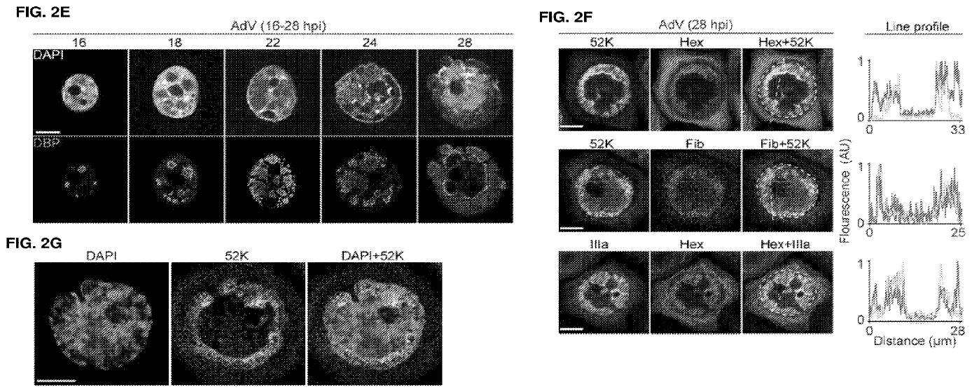

During the late stage of AdV infection, newly synthesized viral DNA genomes are packaged within a proteinaceous icosahedral capsid shell to yield progeny particles approximately 100 nm in dimater¹³⁻¹⁵. However, it is unknown how assembly and packaging are coordinated in the crowded nuclear environment. To investigate the spatial organization of viral processes, we infected human bronchial epithelial cells (HBECs) with AdV serotype 5 (Ad5). Progression to the late phase of infection, when viral late proteins are expressed and progeny virions assembled, was evident by 22 hours post-infection (hpi) (FIGS. 1A and 1B). We reasoned that de novo synthesized viral proteins should become enriched at newly formed sites of viral particle assembly. We used homopropargylglycine (HPG) labelling and fluorophore click chemistry to visualize proteins synthesized within a 30-minute window during the late phase of infection. This labeling approach capitalizes on the fact that during the late phase of infection AdV shuts-off host translation to favor synthesis of viral proteins¹⁶. New protein synthesis was detected in an HPG-dependent manner, with greater mean fluorescence intensity in the nuclei of infected cells compared to uninfected cells (FIGS. 1C and 1D), consistent with high levels of protein synthesis during take-over of host-cell nuclei¹⁶. In infected cells, newly synthesized protein was detected at distinct nuclear sub-compartments (FIG. 1E). These included viral replication compartments (VRCs), hubs of viral genome replication that can be marked by co-staining with antibody to viral DNA binding protein (DBP)^{12,17,18} (FIG. 2). In addition, we observed striking enrichment of newly synthesized protein at nuclear bodies (NBs) of unknown function which we termed viral late nuclear bodies (VLNBs) (FIG. 1E).

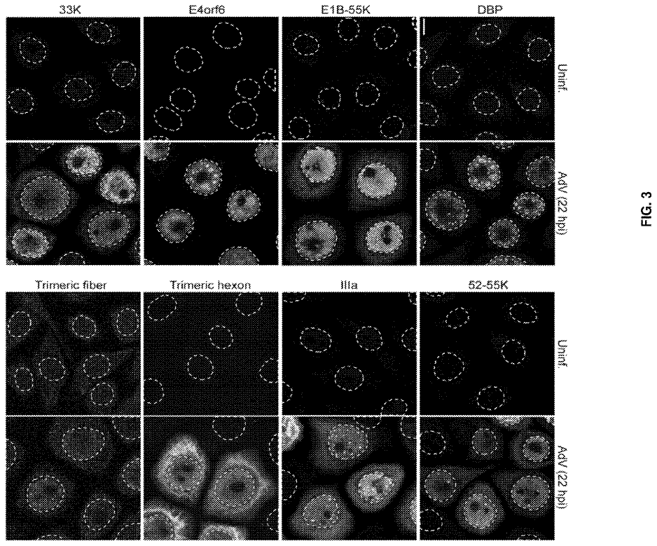

We next investigated the sub-cellular localization of representative viral proteins to provide insights into VLNB functions. We found that viral structural proteins examined (cement protein IIIa, hexon, fiber) and the 52K packaging protein localized to VLNBs (FIG. 1F; FIG. 3). In contrast, other early or late-expressed proteins analyzed (DBP, E4orf6, E1B55K, and 33K) did not localize to VLNBs, with DBP and E4orf6 localized to VRCs (FIG. 3). Co-staining revealed that structural proteins and 52K are all present at the same structures visualized by HPG labelling and confirmed that they are not VRCs (FIGS. 1F, IG; FIG. 2A). Many smaller foci were observed at the peripheral replicative zone (PRZ) that surrounds the core of VRCs (FIG. 1G). The PRZ is the sites of viral genome replication, as well as a proposed site of progeny assembly, nucleated by newly replicated genomes^{12,13,19}. To better understand VLNB formation in the context of virus-induced nuclear reorganization, we used 52K as a marker of VLNBs for phenotype analysis over a time-course of infection that captures transition into the late phase of infection (16-28 hpi). Diffuse nuclear accumulation of 52K (phenotype I) preceded formation of punctate VLNBs (II), which in turn preceded an intermediate non-punctate localization phenotype (III), and eventual accumulation at the nuclear periphery (IV) (FIGS. 1H, 1I). Progression of these phenotypes coincided with the late phase of infection, indicated by increasing abundance of viral late proteins (FIG. 1A), the production of infectious progeny (FIG. 1B), and changes in nucleus size, nucleus morphology, and VRC organization characteristic of the late phase of infection (FIG. 2A-2F)^{12,18}. For clarity, we designated phenotypes II and IV as punctate or peripheral VLNBs, respectively. Co-staining indicated that peripheral VLNBs also contain structural proteins (FIG. 2F). We observed that peripheral bodies are excluded from the center of the nucleus (FIG. 1H, FIG. 2F), which was previously shown to exist as a solid compartment impermeable to immunostaining and ²⁰. We also found that in many cases 52K within peripheral VLNBs exhibited a non-uniform, mottled distribution (FIG. 2G), suggesting that VLNBs break down or become interspersed with accumulated particles that exclude 52K. We conclude that VLNBs, which contain viral structural proteins and the 52K packaging protein, are formed as rounded droplet-like bodies that undergo dynamic changes in size and morphology coincident with progeny production and reorganization of the nucleus.

### Viral Late Nuclear Bodies Exhibit Dynamic Liquid-Like Behavior.

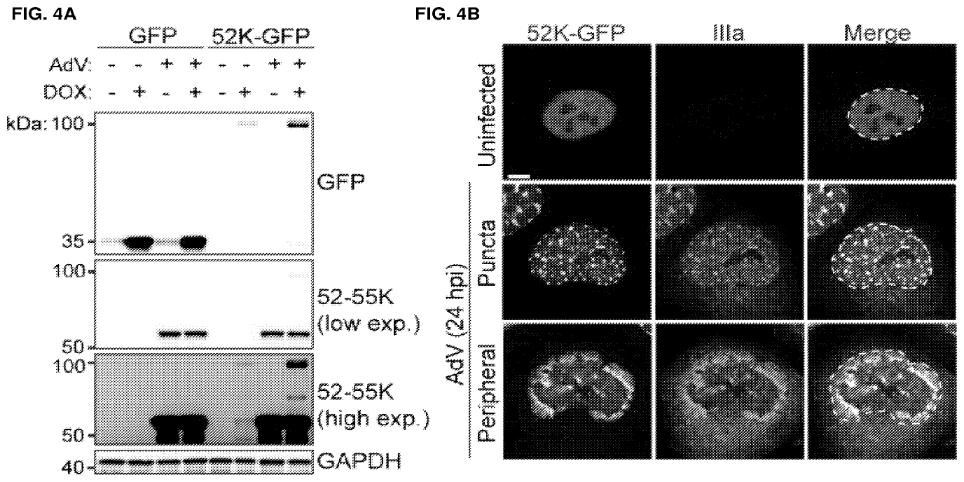

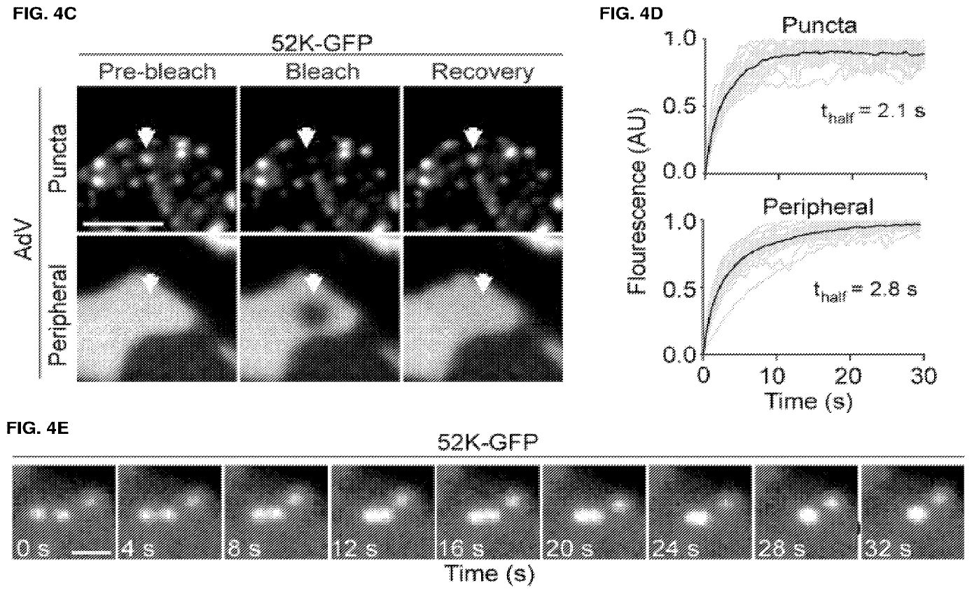

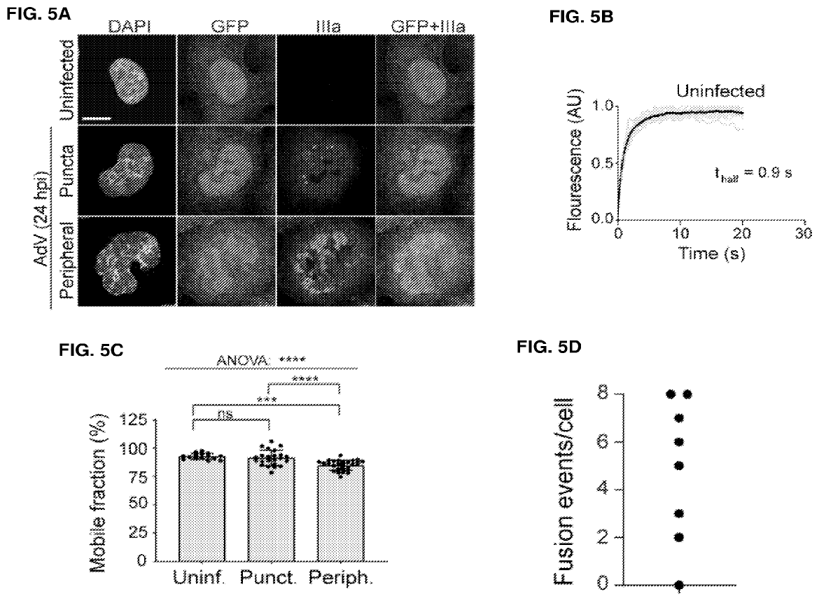

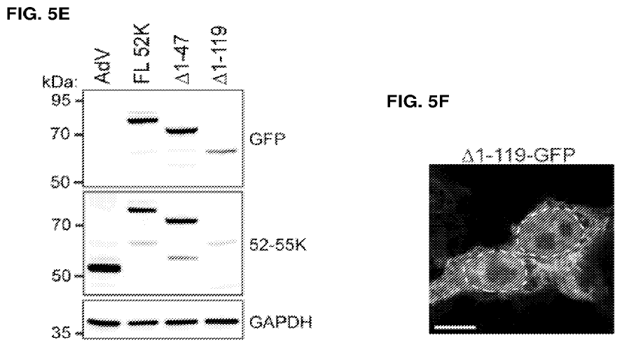

Phase-separated BMCs may exhibit dynamic liquid-like behavior when observed in live cells^{2,21}. To investigate VLNB dynamics in live cells, we engineered a lung cell line (A549) to express GFP-tagged 52K when induced by doxycycline, at levels lower than observed during infection (FIG. 4A). Fluorescent 52K-GFP did not form NBs in uninfected cells, instead localizing diffusely throughout the nucleus (FIG. 4B). In infected cells, 52K-GFP localized into VLNBs, confirmed by colocalization with endogenous IIIa (FIG. 4B), whereas GFP control did not (FIG. 5A). In live cells, Fluorescence recovery after photobleaching (FRAP) of punctate VLNBs (half times=2.1 s) or peripheral VLNBs (half times=2.8 s) was fast, and only moderately slower than that of diffuse 52K in uninfected cells (half time=0.9 s) (FIGS. 4C, 4D; FIG. 5B. In all cases, recovery of fluorescence was high (˜90%) (FIG. 5C). These data indicate rapid movement of 52K between punctate VLNBs and the nucleoplasm, or within peripheral VLNBs. Live cell imaging revealed that punctate VLNBs could be observed to fuse together upon contact like liquid-droplets (FIG. 4E; FIG. 5D). We conclude that VLNBs exhibit dynamic liquid-like behavior.

The 52K Protein is Necessary and Sufficient for Phase-Separation which Requires its Intrinsically Disordered Region (IDR).

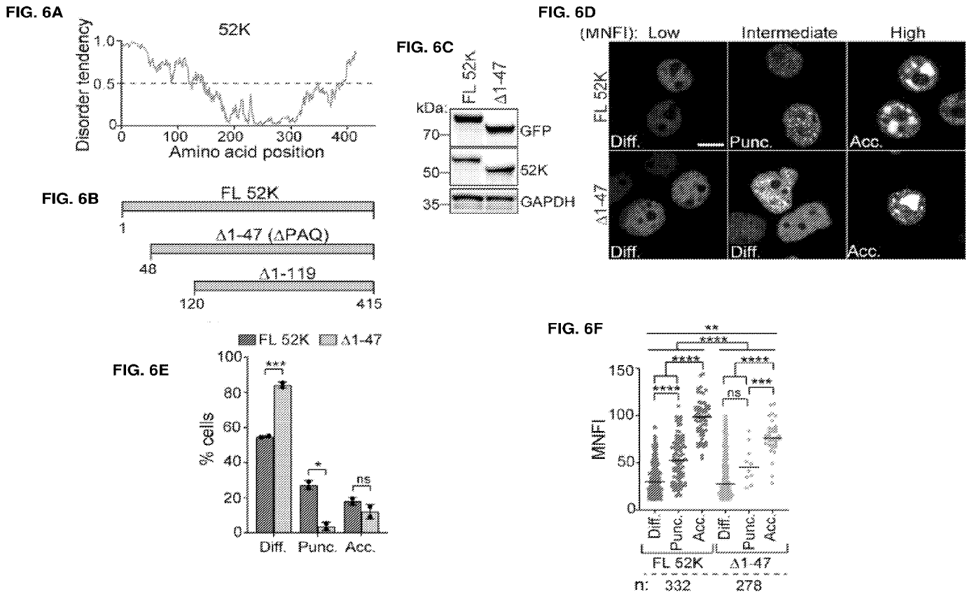

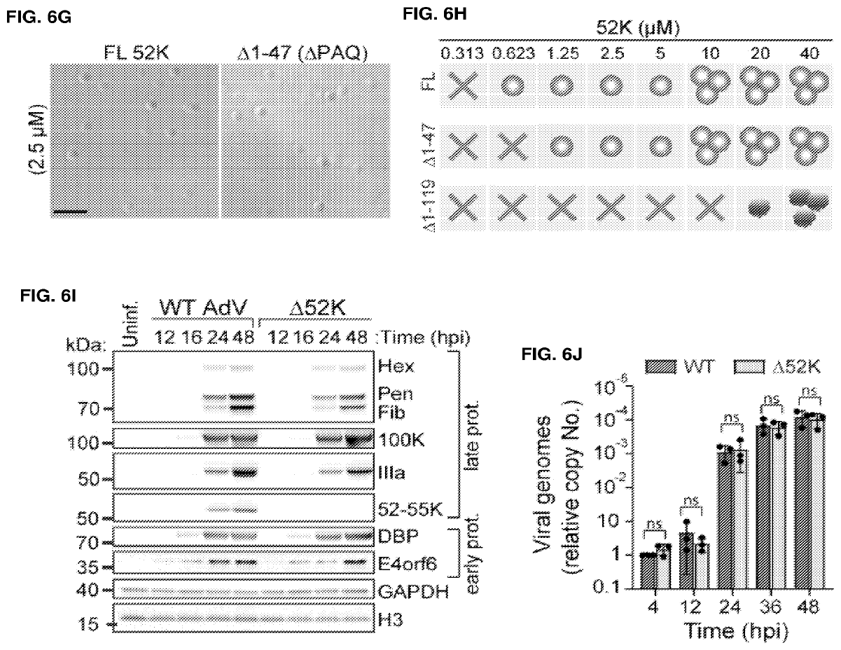

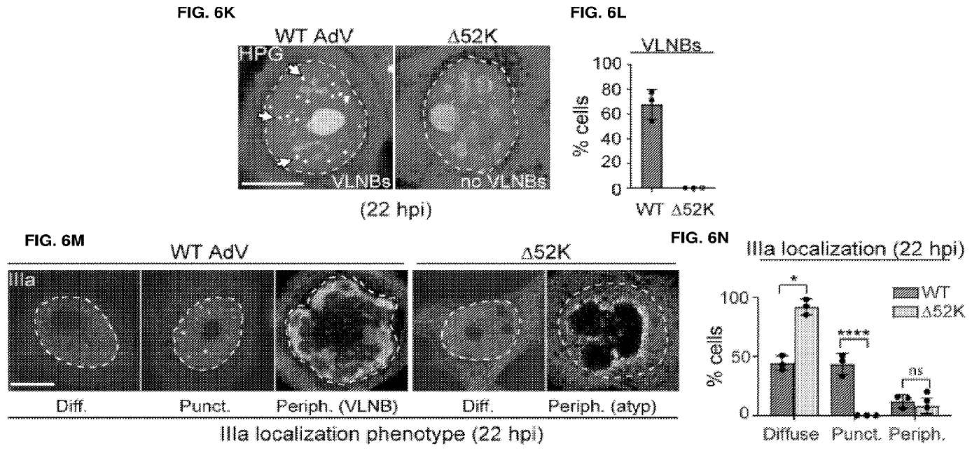

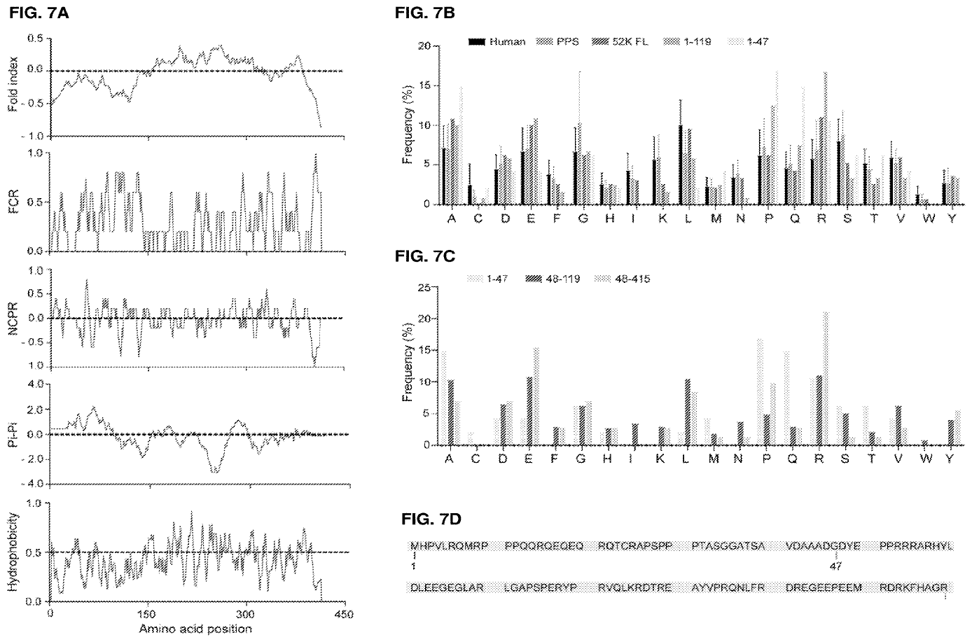

Given that the 52K protein is present at VLNBs, and is known to interact directly or indirectly with structural proteins and viral genomes^{14,15}, we reasoned that 52K plays a central role in VLNB formation. To explore this possibility, we first analyzed the amino acid sequence of 52K for features common in phase-separating proteins^{2,21,22}. Analysis of disorder tendency and fold-index identified a predicted N-terminal intrinsically disordered region (IDR) of approximately 119 amino acids (FIG. 6A; FIG. 7A). This region demonstrated several features proposed to contribute to protein phase-separation including local regions of net charge, a predicted tendency for π-π interactions, and small hydrophobic stretches (FIG. 7A). The IDR exhibited an amino acid composition bias, with arginine and proline, which are common in phase-separating proteins^{22,23}, over-represented compared to both the human proteome and full length (FL) 52K (FIG. 7B). Interestingly, the extreme N-terminal region of the IDR corresponding to amino acids 1-47, which had a particularly high disorder tendency, contained a high proportion of proline, alanine, and glutamine (FIG. 7B-7D). We therefore designated this the proline, alanine and glutamine rich (PAQ) region.

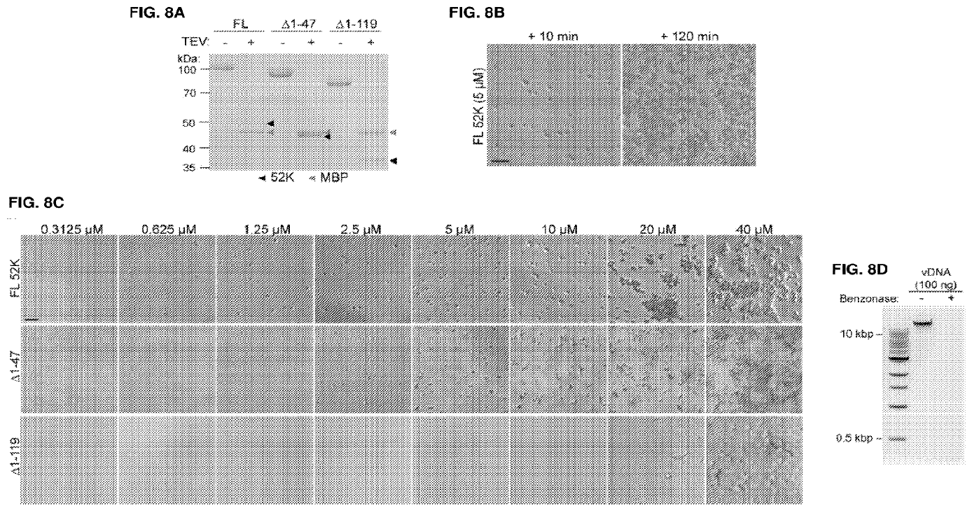

To explore further the role of the IDR, we generated GFP-tagged FL 52K or N-terminal truncation mutants (Δ1-47, Δ1-119) (FIG. 6B). When expressed independent of infection in 293T cells, abundance of the FL 52K and Δ1-47 proteins was comparable to abundance of endogenous 52K in infected cells (FIG. 6C; FIG. 5E). In the absence of infection, the FL 52K protein formed numerous small NBs resembling those present during infection, as well as larger atypical accumulates (FIGS. 6D, 6E). Formation of NBs correlated with expression level indicated by mean nuclear fluorescence intensity (MNFI) (FIG. 3F), consistent with reported concentration-dependent phase-separation in cells²⁴. The Δ1-47 truncated protein formed fewer punctate NBs compared to FL, instead forming mostly larger atypical accumulates (FIG. 6D-6F). The Δ1-119 protein did not form NBs at all, although this mutant also exhibited lower levels of expression and attenuated nuclear localization compared to FL 52K and Δ1-47 (FIG. 5E, 5F). We next assessed the ability of 52K or the IDR-mutants Δ1-47 and Δ1-119 to undergo phase-separation in vitro. The 52K protein was expressed in E. coli and purified with maltose binding protein (MBP) fused at the N-terminus to prohibit phase-separation prior to cleavage by TEV protease (FIG. 8A)²⁵. FL 52K formed condensates at concentrations of 0.625 μM and above (FIG. 6G, 6H; FIG. 8C). These condensates were prone to bunching, particularly at high concentrations, and formed amorphous bunched aggregates when incubated for a further 2 h (FIG. 8B, 8C), consistent with the behavior of gel-like condensates^{21,26-28}. This suggests that the highly dynamic behavior of VLNBs observed in infected cells may require additional viral or cellular factors. The Δ1-47 mutant formed condensates similar in appearance to those of FL 52K, requiring only a moderately higher concentration compared to FL 52K (FIG. 6G, 6H, FIG. 8C). In contrast, Δ1-119 did not form rounded condensates but aggregated at high concentrations (FIG. 6H; FIG. 8C), indicating that the 52K protein IDR contributes to phase-separation.

Given that the 52K protein is sufficient for phase-separation, we reasoned that it may function as an essential driver of VLNB formation. To investigate, we infected HBECs with a viral mutant that does not express 52K (Δ52K) and assessed VLNB formation by visualization of newly synthesized proteins or immunostaining of cement protein IIIa. Although viral proteins and genomes accumulated to levels comparable to WT virus (FIG. 6I, 6J), VLNBs did not form in cells infected with the Δ52K mutant (FIG. 6K-6N). This confirmed that 52K is essential for VLNB formation. We conclude that the viral 52K protein is necessary and sufficient for formation of phase-separated BMCs.

### The 52K Protein PAQ Region is Essential for DNA-Mediated Modulation of Viral Condensates.

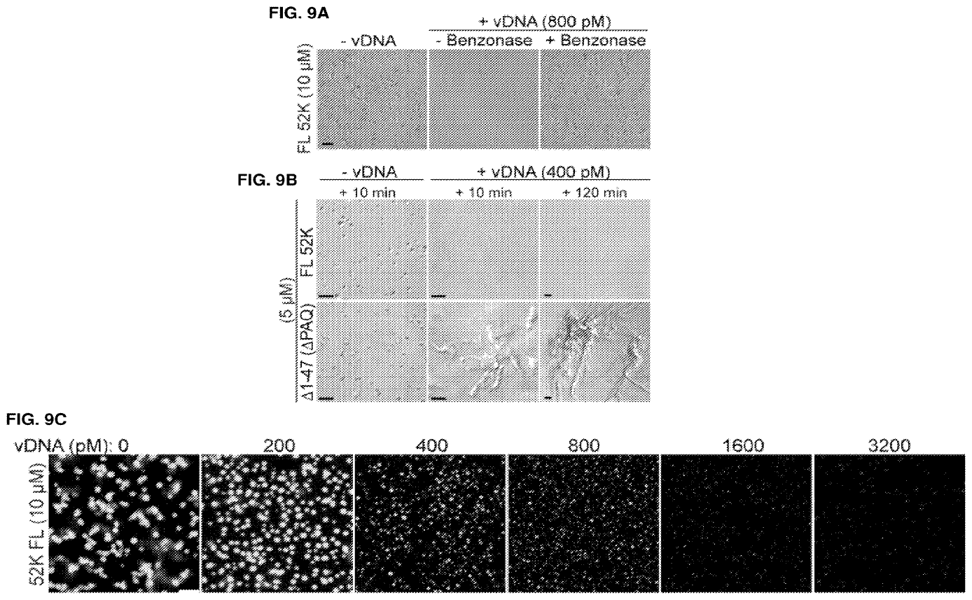

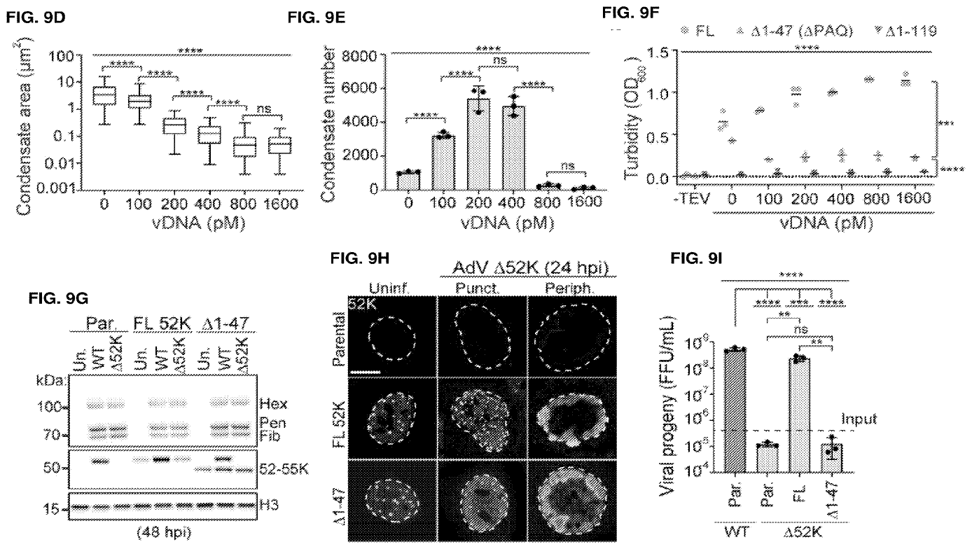

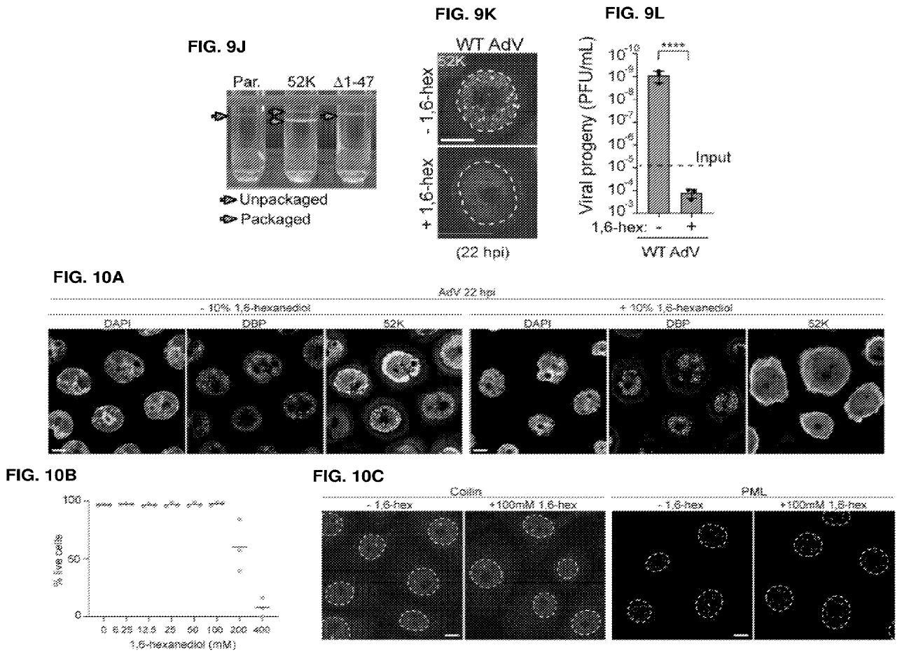

The 52K protein is known to interact with both viral and non-viral DNA^{14,29,30}, raising the question of how viral DNA genomes may modulate viral condensates. When added to phase-separation assays, dsDNA corresponding to the full 36 kb Ad 5 genome (vDNA) decreased the size of 52K condensates, which although no longer readily observable by phase contrast microscopy, could be better visualized by confocal microscopy (FIGS. 9A-9D; FIG. 8D). There was no impact on viral condensates when vDNA was pre-treated with benzonase (FIGS. 9A, 8D), confirming that modulation requires polymeric DNA. The FL 52K condensates formed in the presence of vDNA did not bunch together (FIGS. 9A-9C), but instead could be observed to fuse together upon contact in a liquid-like manner (data not shown). Addition of vDNA at low concentrations (100-400 μM) increased the number of condensates observed, whilst at higher concentrations (800-1600 μM) fewer condensates were visible (FIGS. 9C, 9E). Turbidity of the solution increased with DNA concentration (FIG. 9F), indicating that although fewer condensates were large enough to be visualized by microscopy at high DNA concentrations, nano-scale assemblies of 52K capable of scattering light were still abundant. We note that these nano-scale assemblies are reminiscent of electron-dense putative assembly intermediates containing 52K, previously identified by electron microscopy¹⁹. In contrast, when vDNA was added to phase-separation assays of Δ1-47, large fibrous aggregates, which were never observed with FL 52K, formed over time (FIG. 9B), while turbidity of the solution decreased (FIG. 9F). We conclude that the N-terminal PAQ region of the 52K protein IDR (amino acids 1-47) is essential for DNA-mediated modulation of viral condensates that gives rise to nano-scale assemblies of 52K.

### Viral Late Nuclear Bodies and the 52K Protein PAQ Region are Essential for Production of Infectious Progeny Containing Viral Genomes.

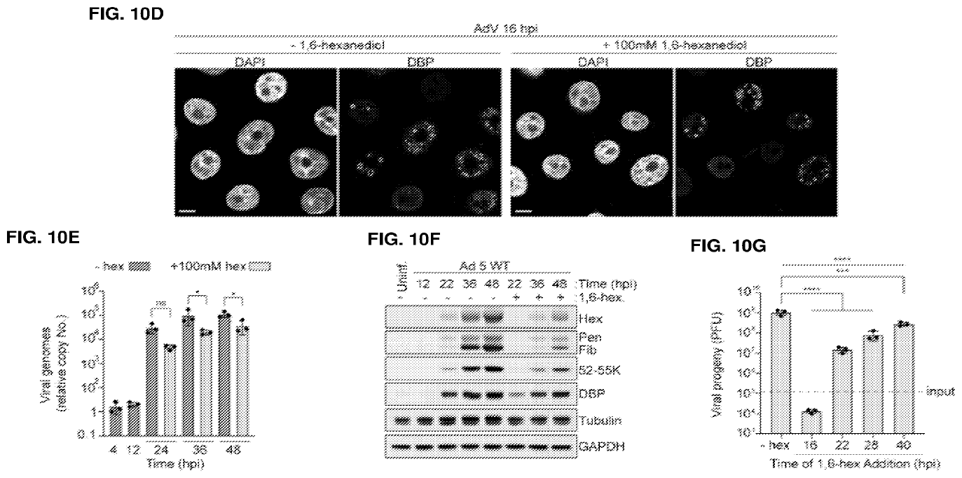

Although not itself a component of mature virions, the 52K protein is required to produce packaged particles containing viral genomes^{14,31,32}. We hypothesized that phase-separation of structural proteins and modulation of condensates by viral genomes may function to coordinate assembly of packaged particles. We therefore assessed the ability of FL 52K or Δ1-47 to complement the Δ52K mutant virus when expressed in trans. In parental A549 cells that do not express 52K (FIG. 9G), no VLNBs formed during Δ52K infection, and only non-infectious empty capsids devoid of genomes accumulated (FIGS. 9H, 9J), which manifest as a lower density band when analyzed by cesium chloride gradient centrifugation^{15,31}. When expressed in uninfected or Δ52K infected cells, both FL 52K & Δ1-47 formed VLNBs (FIGS. 4G, 4H). However, smaller, more numerous VLNBs indicative of DNA-mediated modulation could only be observed in infected cells expressing FL 52K (FIG. 9H). Reconstitution of VLNB formation by FL 52K resulted in rescue of genome packaging and infectious progeny production (FIGS. 9G, 9J), indicating that VLNB formation is essential for progeny production. In contrast, Δ1-47 failed to rescue assembly of packed particles and infectious progeny production (FIGS. 9G-9J). This indicates that VLNB formation alone is not sufficient for progeny production, and that the 52K protein PAQ region, which contributes to DNA-mediated modulation of condensates, is required for packaging of genomes into particles. To further evidence a role for VLNBs in packaging, we next assessed the impact of 1,6-hexanediol, which can disrupt certain BMCs when added to cells at high concentrations²¹. When added to cells infected with WT AdV, 10% 1,6-hex was sufficient to disrupt VLNBs and re-localize 52K to diffuse nuclear and cytoplasmic locations (FIG. 10A). In contrast, VRCs were not disassembled (FIG. 10A), suggesting there may be underlying differences in the interactions that underpin these different viral compartments. When cells were incubated with a much lower concentration of 1,6-hex (1.18% w/v; 100 mM), plasma membranes, PML NBs, and Cajal bodies remained intact (FIGS. 10B, 10C). When infected cells were incubated under these low concentration conditions, VRCs formed (FIG. 10D), and viral genomes and late proteins accumulated, albeit to attenuated levels indicating that infection is viable (FIGS. 10E, 10F). However, VLNB formation was entirely prevented, and infectious progeny production completely abolished (FIGS. 9K, 9I; FIG. 10G), further linking VLNBs formation and progeny production. We conclude that assembly of packaged particles containing viral genomes requires formation of phase-separated BMCs and the 52K PAQ region involved in DNA-mediated modulation of viral condensates.

### Discussion.

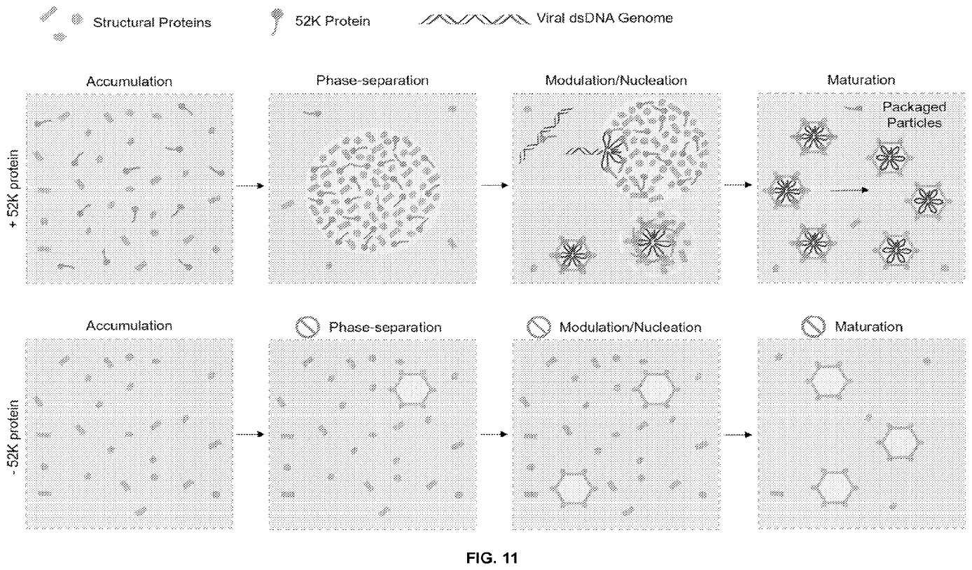

The 52K protein of AdV has been proposed as the link between capsid assembly and genome packaging^{14,15} This implication came from data demonstrating that 52K interacts directly or indirectly with structural proteins and viral genomes, co-purifies with immature particles, and is essential for production of infectious progeny^{14,15,30-33}. Our data indicate that 52K-mediated phase-separation serves to organize viral structural proteins to coordinate assembly of infectious progeny. The accumulation of empty, non-infectious particles in the absence of VLNBs suggests that phase separation of structural proteins is not required for assembly of the particle's protein shell. This is expected given that the in vitro formation of empty viral pseudo-particles from minimal constituent components in solution is well known. Instead, our data supports a model in which assembly of packaged particles is nucleated by DNA-mediated modulation of BMCs (FIG. 11).

Viral membrane-less compartments typically harbor multiple viral processes, which in some cases localize to dedicated sites within compartments suggesting a higher level of organization^{3,4,6,12} It is possible that segregation of viral processes promotes replication by providing distinct environments conducive to specific processes³⁴. Consistent with this proposition, we find that viral structural proteins form 52K-dependent VLNBs distinct from VRCs. The interaction of 52K with viral genomes and structural proteins, and its temporary incorporation into assembling particles suggests that 52K functions to nucleate concurrent assembly and packaging^{15,19}. The short N-terminal PAQ region of the viral 52K protein is enriched for both dispersed proline residues which are implicated in the promotion of a liquid state²², and glutamine, which can promote liquid to solid transitions³⁵. We find that the PAQ region is essential for both genome packaging and the modulation of viral condensates by DNA. Thus, it appears that the PAQ region plays a critical role in mediating transitions in material state required for the nucleation of packaged particles from liquid-like phase-separated structural proteins.

Others have demonstrated that the nucleoproteins of RNA viruses such as SARS-CoV-2, measles virus, and HIV, phase-separate with viral RNA, implicating phase-separation in multiple viral processes including the spatial organization and compaction of viral RNA genomes required for packaging and assembly^{3-5,8,9,11,36}. Unlike these RNA viruses, the AdV proteins involved in particle assembly and genome packaging are distinct from those involved in transcription and replication of the viral genome¹³⁻¹⁵. Although essential for production of packaged particles, the 52K protein is otherwise dispensable for viral replication^{14,32}. By breaking and restoring phase-separation via complementation of a 52K-null mutant virus, we provide the first direct evidence that phase-separation is essential for production of packaged progeny particles in virus-infected cells. Together, these emerging findings suggest that phase-separation may represent a common strategy utilized by diverse viruses, pertinent where assembly of the progeny particle is nucleated by the viral genome. Given the inherently virus-specific nature of progeny assembly, and the essential requirement of 52K-driven phase-separation, our findings highlight AdV infection as a promising target of condensate-modulating small molecules, and an attractive system for investigation of strategies that target phase-separation for wider therapeutic gain.

### REFERENCES

1. Hyman, A. A., Weber, C. A. & Julicher, F. Liquid-liquid phase separation in biology. Annu. Rev. Cell Dev. Biol. 30, 39-58 (2014).
2. Shin, Y. & Brangwynne, C. P. Liquid phase condensation in cell physiology and disease. Science 357, eaaf4382 (2017).
3. Etibor, T. A., Yamauchi, Y. & Amorim, M. J. Liquid Biomolecular Condensates and Viral Lifecycles: Review and Perspectives. Viruses 13, (2021).
4. Su, J. M., Wilson, M. Z., Samuel, C. E. & Ma, D. Formation and Function of Liquid-Like Viral Factories in Negative-Sense Single-Stranded RNA Virus Infections. Viruses 13, (2021).
5. Sengupta, P. & Lippincott-Schwartz, J. Revisiting Membrane Microdomains and Phase Separation: A Viral Perspective. Viruses 12, (2020).
6. Risso-Ballester, J. et al. A condensate-hardening drug blocks RSV replication in vivo. Nature 595, 596-599 (2021).
7. Heinrich, B. S., Maliga, Z., Stein, D. A., Hyman, A. A. & Whelan, S. P. J. Phase Transitions Drive the Formation of Vesicular Stomatitis Virus Replication Compartments. MBio 9, e02290-17 (2018).
8. Iserman, C. et al. Genomic RNA Elements Drive Phase Separation of the SARS-CoV-2 Nucleocapsid. Mol Cell 80, 1078-1091.e6 (2020).
9. Guseva, S. et al. Measles virus nucleo- and phosphoproteins form liquid-like phase-separated compartments that promote nucleocapsid assembly. Sci Adv 6, (2020).
10. McSwiggen, D. T. et al. Evidence for DNA-mediated nuclear compartmentalization distinct from phase separation. Elife 8, e47098 (2019).
11. Cubuk, J. et al. The SARS-CoV-2 nucleocapsid protein is dynamic, disordered, and phase separates with RNA. Nat Commun 12, 1936 (2021).
12. Charman, M. & Weitzman, M. D. Replication Compartments of DNA Viruses in the Nucleus: Location, Location, Location. Viruses 12, (2020).
13. Charman, M., Herrmann, C. & Weitzman, M. D. Viral and Cellular Interactions During Adenovirus DNA Replication. FEBS Lett. 593, 3531-3550 (2019).
14. Ahi, Y. S. & Mittal, S. K. Components of Adenovirus Genome Packaging. Front Microbiol 7, 1503 (2016).
15. Condezo, G. N. et al. Structures of Adenovirus Incomplete Particles Clarify Capsid Architecture and Show Maturation Changes of Packaging Protein L1 52/55k. J Virol 89, 9653-9664 (2015).
16. Russell, W. C. & Skehel, J. J. The polypeptides of adenovirus-infected cells. J Gen Virol 15, 45-57 (1972).
17. Pombo, A., Ferreira, J., Bridge, E. & Carmo-Fonseca, M. Adenovirus replication and transcription sites are spatially separated in the nucleus of infected cells. EMBO J. 13, 5075-5085 (1994).
18. Pied, N. & Wodrich, H. Imaging the adenovirus infection cycle. FEBS Lett. 593, 3419-3448 (2019).
19. Condezo, G. N. & San Martin, C. Localization of adenovirus morphogenesis players, together with visualization of assembly intermediates and failed products, favor a model where assembly and packaging occur concurrently at the periphery of the replication center. PLoS Pathog. 13, e1006320 (2017).
20. Pfitzner, S. et al. Fluorescent protein tagging of adenoviral proteins pV and pIX reveals ‘late virion accumulation compartment’. PLOS Pathogens 16, e1008588 (2020).
21. Alberti, S., Gladfelter, A. & Mittag, T. Considerations and Challenges in Studying Liquid-Liquid Phase Separation and Biomolecular Condensates. Cell 176, 419-434 (2019).
22. Gomes, E. & Shorter, J. The molecular language of membraneless organelles. J Biol Chem 294, 7115-7127 (2019).
23. van Mierlo, G. et al. Predicting protein condensate formation using machine learning. Cell Rep 34, 108705 (2021).
24. Riback, J. A. et al. Composition-dependent thermodynamics of intracellular phase separation. Nature 581, 209-214 (2020).
25. Alberti, S. et al. A User's Guide for Phase Separation Assays with Purified Proteins. J Mol Biol 430, 4806-4820 (2018).
26. Riback, J. A. et al. Stress-Triggered Phase Separation Is an Adaptive, Evolutionarily Tuned Response. Cell 168, 1028-1040.e19 (2017).
27. Patel, A. et al. A Liquid-to-Solid Phase Transition of the ALS Protein FUS Accelerated by Disease Mutation. Cell 162, 1066-1077 (2015).
28. Lin, Y., Protter, D. S. W., Rosen, M. K. & Parker, R. Formation and Maturation of Phase-Separated Liquid Droplets by RNA-Binding Proteins. Mol Cell 60, 208-219 (2015).
29. Perez-Romero, P., Tyler, R. E., Abend, J. R., Dus, M. & Imperiale, M. J. Analysis of the interaction of the adenovirus L1 52/55-kilodalton and IVa2 proteins with the packaging sequence in vivo and in vitro. J. Virol. 79, 2366-2374 (2005).
30. Zhang, W. & Arcos, R. Interaction of the adenovirus major core protein precursor, pVII, with the viral DNA packaging machinery. Virology 334, 194-202 (2005).
31. Hasson, T. B., Soloway, P. D., Ornelles, D. A., Doerfler, W. & Shenk, T. Adenovirus L1 52- and 55-kilodalton proteins are required for assembly of virions. J. Virol. 63, 3612-3621 (1989).
32. Gustin, K. E. & Imperiale, M. J. Encapsidation of Viral DNA Requires the Adenovirus L1 52/55-Kilodalton Protein. Journal of Virology 72, 7860-7870 (1998).
33. Ma, H.-C. & Hearing, P. Adenovirus Structural Protein IIIa Is Involved in the Serotype Specificity of Viral DNA Packaging v. J Virol 85, 7849-7855 (2011).
34. Fare, C. M., Villani, A., Drake, L. E. & Shorter, J. Higher-order organization of biomolecular condensates. Open Biol 11, 210137.
35. Wang, J. et al. A Molecular Grammar Governing the Driving Forces for Phase Separation of Prion-like RNA Binding Proteins. Cell 174, 688-699.e16 (2018).
36. Alenquer, M. et al. Influenza A virus ribonucleoproteins form liquid organelles at endoplasmic reticulum exit sites. Nat Commun 10, (2019).

### Example 2

Nanoparticles Comprising p52K and a Nucleic Acid of Interest for Expression of Said Nucleic Acid in Cells

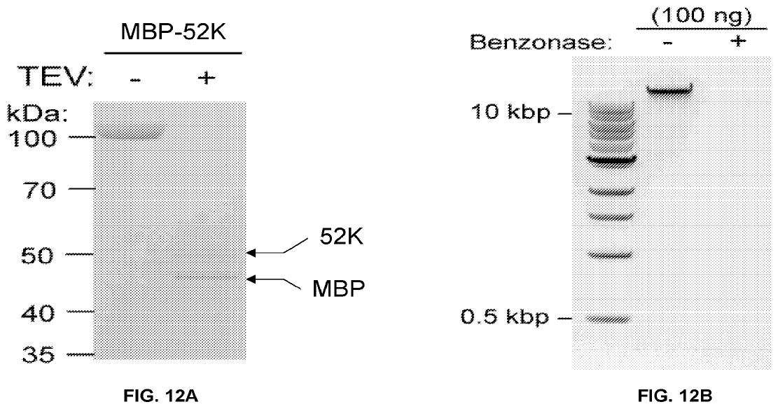

FIG. 12A is a coomassie gel showing purified MBP-52K and its cleavage by TEV protease (into MBP & 52K respectively). This is the process used to enable assembly of the nanoparticles described herein. MBP-52K was expressed in E. coli, purified and buffer exchanged/concentrated into phase-separation buffer. Protein was incubated for 1 h at room temperature with (+) or without (−) TEV protease. Proteins were separated by SDS-PAGE and stained with Coomassie. FIG. 12B is an agarose gel showing purified viral genomes (vDNA). The Benzonase treatment confirms that this is nucleic acid. Plasmid DNA was amplified in bacteria and purified. Viral genomes (vDNA) were excised from plasmid DNA by restriction digest, separated by gel electrophoresis, purified, and buffer exchanged. Purified vDNA was analyzed by gel electrophoresis and stained with ethidium bromide.

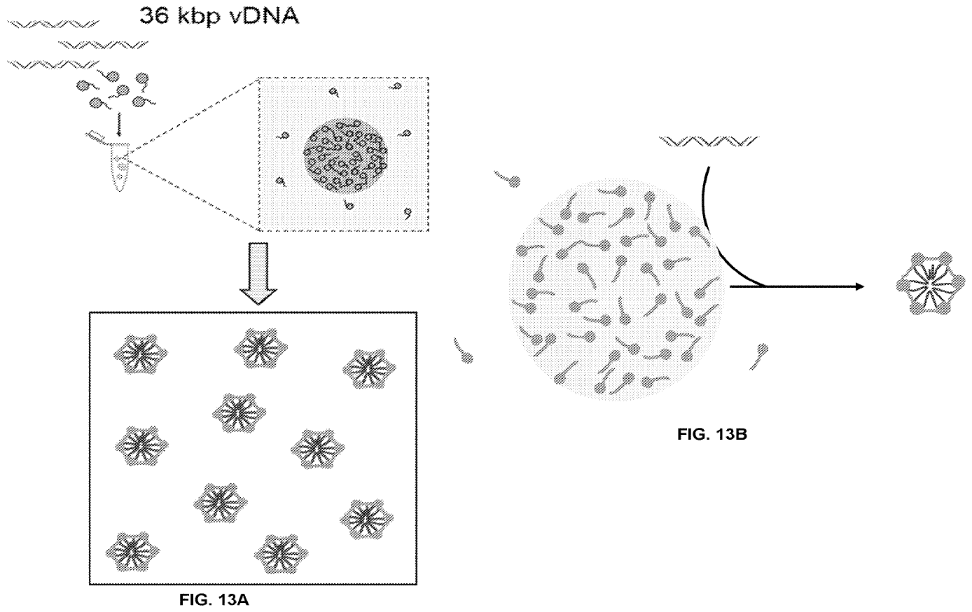

In vitro phase-separation assays were carried out in phase-separation buffer (25 mM Tris-HCl, 150 mM NaCl, 1 mM DTT, pH 7.4) using the indicated concentration of proteins and DNA in a final volume of 25 μL. TEV protease was added at w/w ratio of 1:50, and the reaction incubated at 25° C. for 30 minutes prior to analysis. For visualization, the sample was transferred to a 384 well plate and imaged by confocal microscopy. Turbidity of samples was analyzed by O.D600 using NanoDrop One spectrophotometer (Thermo Fischer Scientific) as per the manufacturer's guidelines. FIG. 13 depicts 2 schematics showing the assembly process. MBP-52K and vDNA are combined in the same tube and TEV protease is added to release 52K from MBP and induce self-assembly of nanoscale assemblies. We refer to this process as “in vitro phase-separation assay”, or in the alternative as an “in vitro assembly assay”.

These data characterize what happens when we induce self-assembly in the presence of different concentrations of vDNA.

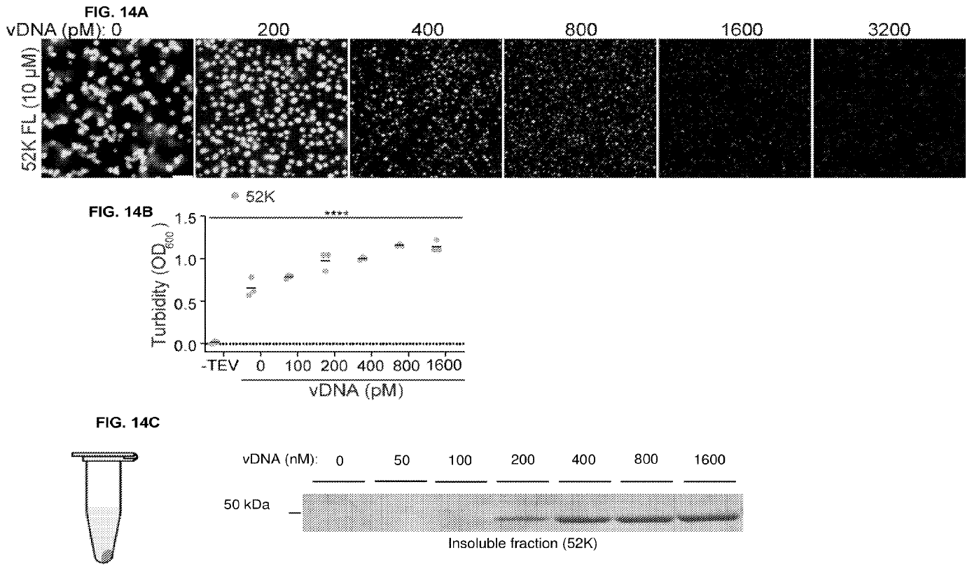

The MBP-52K used in the assembly was fluorescently labelled and visualized post-assembly by confocal microscopy. When high concentrations of vDNA are used the “assemblies” generated are too small to visualize. These assemblies do however contribute to the turbidity of the solution (they scatter light) indicating that they are present in the sample. These assemblies can also be crudely purified in bulk by centrifugation at 2000 g for 3 minutes. The pelleted assemblies can then be analyzed by SDS-PAGE and Coomassie stain. See FIGS. 14A, 14B and 14C.

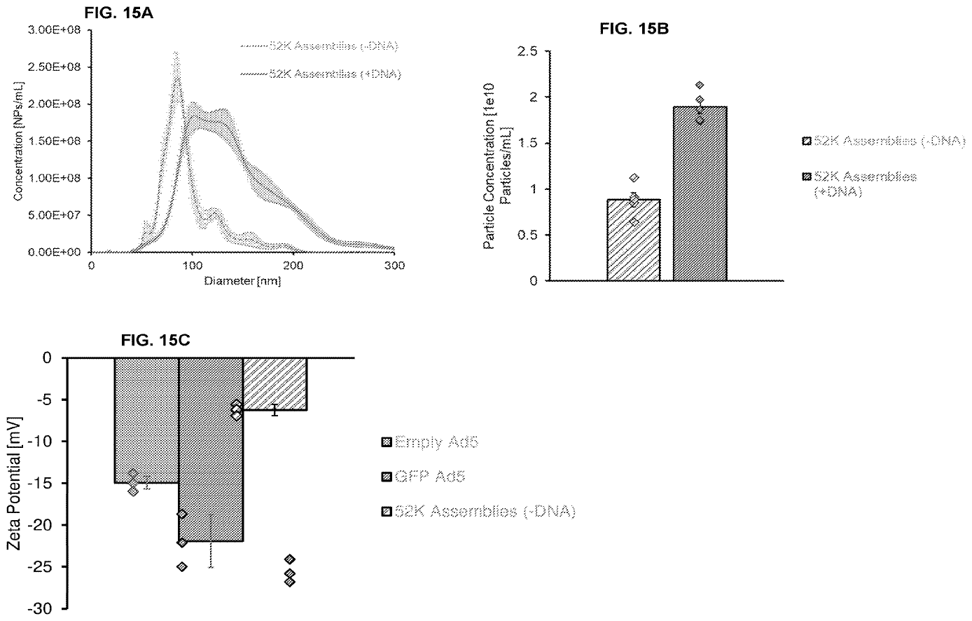

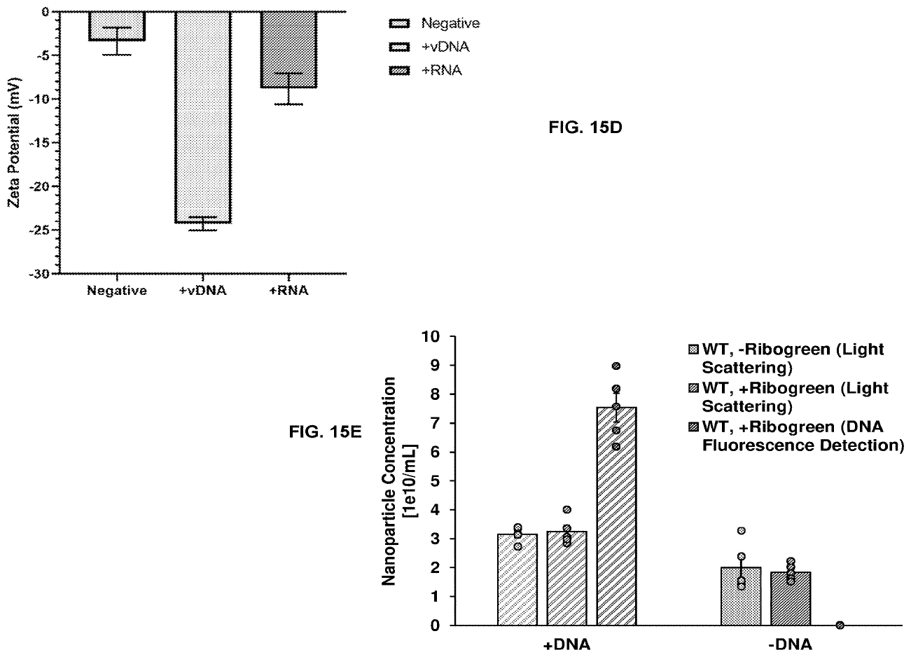

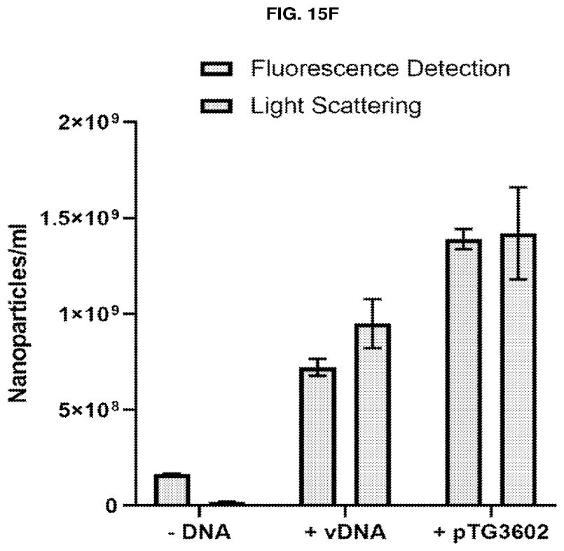

Nanoscale assemblies generated by in vitro phase-separation assays were diluted 1:100 in phase-separation buffer (25 mM Tris-HCl, 150 mM NaCl, 1 mM DTT, pH 7.4) using the indicated concentration of proteins and DNA in a final volume of 25 μL. TEV protease was added at w/w ratio of 1:50, and the reaction incubated at 25° C. for 30 minutes prior to analysis. For visualization, the sample was transferred to a 384 well plate and imaged by confocal microscopy. Turbidity of samples was analyzed by O.D600 using NanoDrop One spectrophotometer (Thermo Fischer Scientific) as per the manufacturer's guidelines. Size distribution and particle number was determined 4 hours post-assembly by nanoparticle tracking analysis using a Malvern NANOSIGHT NS300 (NTA 3.2 Dev Build 3.2.16) using an SOP standard measurement script as per the manufacturer's instructions. Zeta-potential was analyzed using a Malvern Zetasizer as per the manufacturer's instructions. See FIGS. 15A, 15B. FIG. 15C is a graph showing the charge surrounding the particles. The increased negative charge of particles assembled in the presence of vDNA indicated incorporation of the negatively charged DNA. FIG. 15D shows another analysis of the Zeta-potential using the Malvern Zetasizer comparing particles assembled without nucleic acid, with vDNA, or with RNA. FIG. 15E shows another method analyzing the assembled nanoparticles by Nanoparticle Tracking Analysis (NTA) using the NANOSIGHT as described above. RiboGreen, a dye that fluoresces when bound to nucleic acid was used. Nanoparticles were assembled with or without vDNA and were then labeled with the RiboGreen or not labeled as a control. Nanoparticle Tracking Analysis (NTA) was then performed using two different modes. The first mode detects light scattering (as before), the second mode detects fluorescence. FIG. 15F shows circular plasmid DNA can also be incorporated into particles. Nanoparticles were assembled without DNA, with vDNA, or with the circular plasmid pTG3602.

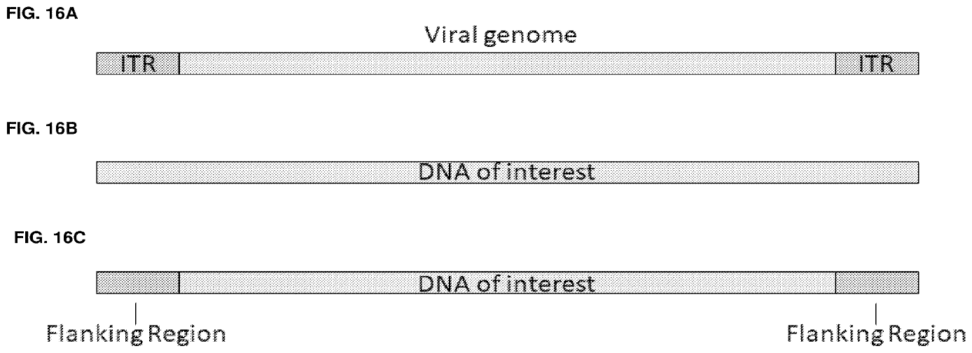

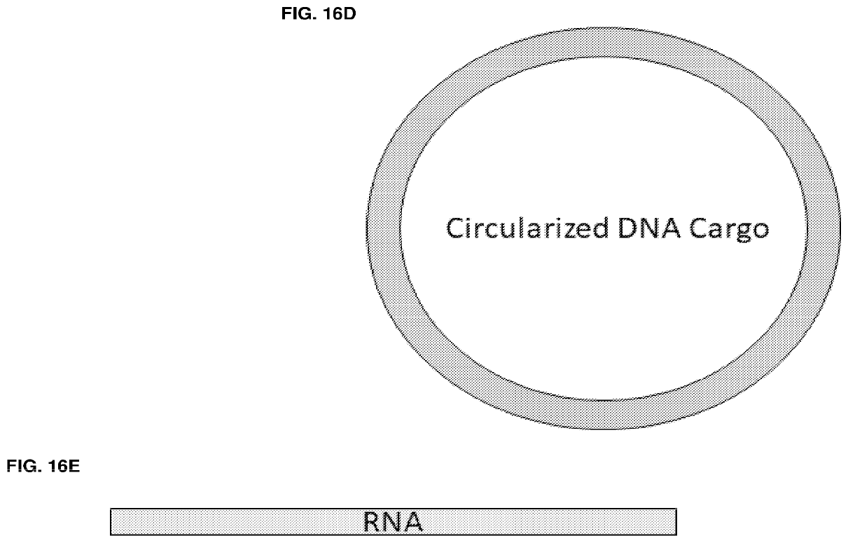

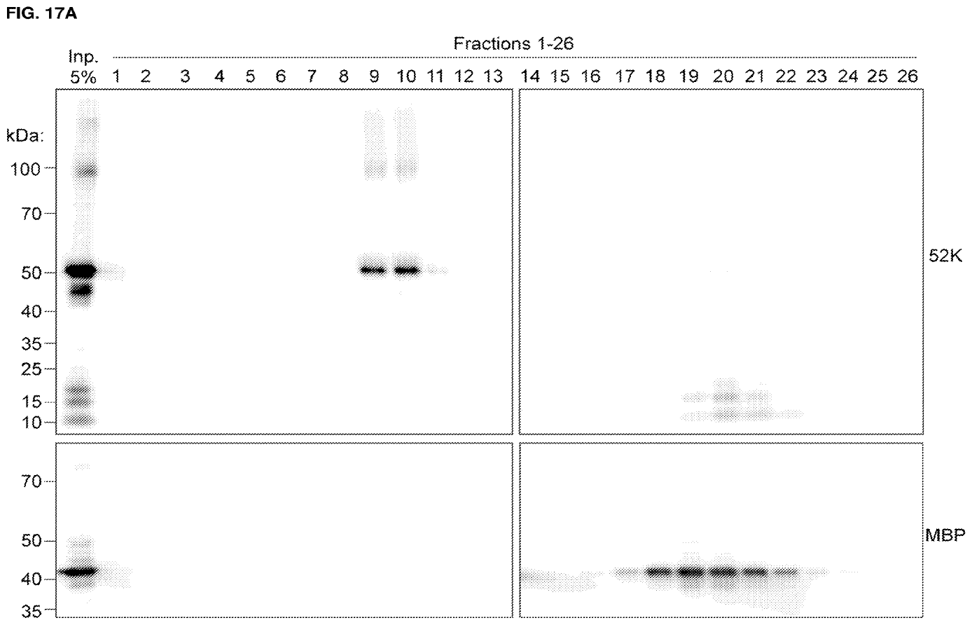

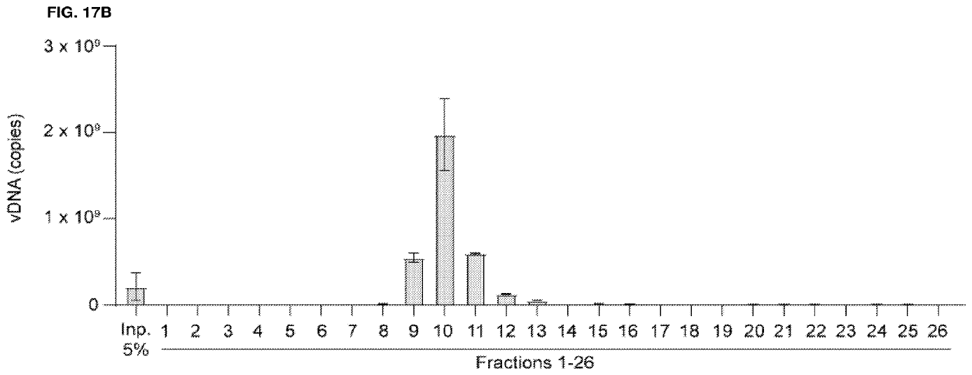

The light scattering data demonstrates that particles assembled in all conditions described and that particles assembled with vDNA can be detected by addition of RiboGreen. Notably, particles assembled without vDNA cannot be detected. Thus, our particles assembled in the presence of vDNA have incorporated the vDNA but the particles assembled without vDNA obviously have not. FIGS. 16A-16E show representative examples of nucleic acid cargo for incorporation into nanoparticles. FIGS. 17A-17B show size exclusion purification of nanoparticles.

The data in FIGS. 12-17 demonstrate that assembly of nanoparticles comprising p52K-DNA complexes occurs under the appropriate conditions. As discussed above in Example 1, viral genome DNA includes ITRs at the 5′ and 3′ ends. ITRs (and more specifically a short packaging sequence in the “left hand” ITR) facilitates packaging of the genome into viral particles during infection. Unlike AAV or AdV vectors (that require real replication for their production), the nanoparticle described herein can be assembled in a tube. Thus, the DNA incorporated into the particle does not need to include viral genes, which can be switched out for whatever is desired. Thus, in certain embodiments, either all, or most (i.e. the central gene-encoding region of the genome but not the ITRs) of the genome could be replaced with the nucleic acid of interest and still successfully incorporate into the nanoparticle. In other approaches, additional viral proteins can be included. Such proteins may or may not be from adenovirus or adeno-associated virus. Proteins of interest include for example, adenovirus major capsid proteins including but not limited to hexon, penton, and fiber, adenovirus minor capsid proteins including but not limited to proteins IIIa, VI, VIII, and IX, adenovirus core proteins including but not limited to terminal protein (TP), Mu, VII, V, adenovirus protease, adenovirus packaging proteins including but not limited to IVa2, 22K, and 33K.

The nucleic acid of interest can be any nucleic acid and includes DNA and RNA. Nucleic acids of interest include without limitation, e.g., other viral genomes, sequences encoding immunogenic proteins for use as a vaccine, or sequences which express a therapeutic protein to alleviate disease symptoms. The DNA to be incorporated can vary is size and can be between 500-2,000 bp, 2000-10,000 bp, 10,000-20,000 bp, 20,000-30,000 bp, 30,000-40,000 bp, 40,000-50,000 bp, and 50,000-100,000 bp in length. Exemplary DNA sequences can include DNA encoding the genome of human adenovirus serotype 5 (NCBI Reference Sequence: AC_000008.1). See the world wide web at .ncbi.nlm.nih.gov/nuccore/ac_000008, for the genome of other adenoviruses. The DNA sequence can also encode genomes of other viruses which are optionally modified by deletion, addition, or substitution. See FIGS. 16A-16D.

The nucleic acid can also include DNA vectors such as plasmids, bacmids, and cosmids, either circular or linearized which further comprise a sequence of interest. In certain approaches the sequence of interest can include one or more ancillary sequences that facilitate expression of nucleic acid of interest, e.g, promoters, leader sequences, polyA tails, and internal ribosome entry sites (IRES). Other ancillary sequences can include sequences that facilitate the cleavage of gene products or encoded RNAs post-expression, including but not limited to recognition sites of cellular or viral proteases or sequence encoding for catalytically active RNA or polypeptide sequences. Also included are sequences encoding functional RNAs, siRNA and shRNA. In certain approaches sequences encoding other RNAs that function as guides or co-factors in gene editing processes, including but not limited to CRISPR Cas9 guide RNAs can be incorporated into the nanoparticle. The nucleic acid of interest can also comprise non-coding sequence that facilitates recombination of the DNA of interest with the cellular (host) genome, including but not limited to sequence derived from retroviruses (including lentiviruses), bacteriophages, or cellular genomes. Other non-coding sequence to be included include those that facilitate circularization of the DNA cargo post-delivery or promote the maintenance of DNA cargo as an episome post-delivery. As noted above, the nucleic acid of interest can be flanked by sequences corresponding to the inverted terminal repeats (ITRs) of an adenovirus genome, or modified forms of the ITRs. Any of the above-mentioned nucleic acid sequences can be modified to limit recognition by the innate immune response.

In preferred embodiments, the nucleic acid of interest encodes a metabolic gene that, when expressed, is capable of rectifying an inherited single gene anomaly, i.e., a single gene coding for an enzyme is defective, and that defect causes an enzyme deficiency. The enzyme deficiency produces an inherited metabolic disease or disorder, of which a subtype is an inborn error of metabolism. Most single gene anomalies are autosomal recessive, i.e., two defective copies of the gene must be present for the disease or trait to develop. Non-limiting examples of metabolic disorders include glucose metabolism disorders, lipid metabolism disorders, malabsorption syndromes, metabolic brain diseases, calcium metabolism disorders, DNA repair-deficiency disorders, hyperlactemia, iron metabolism disorders, metabolic syndrome X, inborn error of metabolism, phosphorus metabolism disorders, and acid-base imbalance. Inherited metabolic diseases previously were classified as disorders of carbohydrate metabolism, amino acid metabolism, organic acid metabolism, or lysosomal storage diseases, however new inherited disorders of metabolism have been discovered and the categories have multiplied. Certain major classes of congenital metabolic diseases include disorders of carbohydrate metabolism, e.g., glycogen storage disease, glucose-6-phosphate dehydrogenase (G6PD) deficiency (resulting from a mutation in the G6PD gene); disorders of amino acid metabolism, e.g., phenylketonuria, maple syrup urine disease, glutaric acidemia type 1; urea cycle disorder (urea cycle defects), e.g., carbamoyl phosphate synthetase I deficiency; disorders of organic acid metabolism (organic acidurias), e.g., alcaptonuria, 2-hydroxyglutaric acidurias; disorders of fatty acid oxidation and mitochondrial metabolism; e.g., medium-chain acyl-coenzyme A dehydrogenase deficiency (often called “MCADD”) (caused by mutations in the ACADM gene, which results in medium-chain fatty acids not being metabolized properly and leads to lethargy and hypoglycemia); disorders of porphyrin metabolism, e.g., acute intermittent porphyria; disorders of purine or pyrimidine metabolism, e.g., Lesch-Nyhan syndrome (caused by mutations in the hypoxanthine phosphoribosyltransferase 1 [HPRT1] gene and inherited in an X-linked recessive manner); disorders of steroid metabolism, e.g., lipoid congenital adrenal hyperplasia, congenital adrenal hyperplasia; disorders of mitochondrial function, e.g., Keams-Sayre syndrome; disorders of peroxisomal function, e.g., Zellweger syndrome (caused by mutations in genes encoding peroxins, e.g., PEX1, PEX2, PEX3, PEX5, PEX6, PEX10, PEX12, PEX13, PEX14, PEX16, PEX19, or PEX26 genes); lysosomal storage disorders, e.g., Gaucher's disease (of which there are three subtypes, all of which are autosomal recessive) and Niemann-Pick disease (has an autosomal recessive inheritance pattern; Niemann-Pick types A and B are caused by a mutation in the Sphingomyelin phosphodiesterase 1 [SMPD1] gene; mutations in NPC1 gene or NPC2 gene cause Niemann-Pick disease, type C [NPC], which affects a protein used to transport lipids; Niemann-Pick type D shares a specific mutation in the NPC1 gene, patients having type D share a common Nova Scotian ancestry).

RNA equivalents of any of the above-mentioned DNA sequences can also be incorporated into the nanoparticle. RNAs of interest can also vary in size and can range from 100-500 nucleotides, 500-1,000 nucleotides, 1000-2,000 nucleotides, 2000-5,000 nucleotides, 5,000-20,000 nucleotides.

During formation of the nanoparticles other components may be included as desired. For example, intrinsically disordered regions (IDRs) of proteins are putative unstructured regions that facilitate protein-driven phase-separation. The data show that the N-terminal IDR of the 52K protein plays an important role in phase-separation as described in Example 1. Thus, in preferred embodiments, IDRs are included in p52K protein to facilitate assembly of the nanoparticles. In certain embodiments, the N-terminal IDR, or other domains in the 52K protein including its C-terminus can be modified to engineer a protein that is improved in its propensity to phase-separate/assemble. In other approaches, the IDRs of 52K are swapped with IDRs from other proteins in order to improve phase-separation/assembly.

Proteins and molecules that bind nucleic acid and help compact, organize, or protect DNA are particularly desirable. These can include proteins or functional fragments thereof derived from histones, protamines, histone-like, protamine-like proteins, enzymes useful in gene editing methods, e.g., Cas9. In certain embodiments, adenovirus protein VII and/or other viral proteins or functional fragments of human adenovirus serotypes that facilitate gene delivery by promoting particle stability, promoting particle disassembly in the cell, promoting escape of the particle from the endosome, or promote transport of the genome to the nucleus can be included in the nanoparticle. Other positively charged viral or cellular proteins that help counteract repulsive negative charge can also form part of the nanoparticle. The nanoparticle can also comprise molecules, both biological and synthetic, that counteract repulsive negative charge.

In certain approaches the nanoparticle can be encapsulated by coatings that stabilize particles or facilitate uptake/entry into cells. For example, lipid monolayers or bilayers comprising a single lipid type or mixtures of lipid types can be included. In a preferred approach, the lipids are positively charged lipids and phospholipids, e.g., a liposome, micelle or other suitable vehicle which facilitates cellular uptake. Natural or synthetic polymers can also be employed and comprise a single polymer type or mixtures of polymer types including but not limited to positively charged polymers.

The liposome, micelle or other vehicle can comprise surface molecules, including without limitation, antibodies, antigen binding regions and FC regions of antibodies from humans and other species, single chain antibodies of camelids and sharks or engineered single chain antibodies (tissue/cell targeting and/or uptake), cell surface receptors or receptor ligands, viral proteins that bind cell surface receptors or receptor ligands and other targeting moieties that promote interaction with cells or tissues. The vehicles can also optionally include a cell penetrating peptide that facilitates targeted uptake of the nanoparticle. Peptides comprising a single peptide type or mixtures of peptide types, e.g., the membrane-penetrating TAT peptide of HIV.

In other approaches, additional viral capsid proteins can be included. Such capsid proteins may or may not be from adenovirus or adeno-associated virus. Proteins of interest include for example, adenovirus major capsid proteins including but not limited to hexon, viral minor capsid proteins including but not limited to proteins IIIa, VI, VIII, and IX, viral core proteins including but not limited to terminal protein (TP), Mu, VII, V, IVa2, adenovirus protease.

With the appropriate nucleic acid cargo and modifications, novel nanoparticles of the invention could be utilized for a variety of indications centered around delivery and expression of a gene or genes. Cells to be treated can be in human patients, veterinary patients, or in a laboratory setting (e.g., expression in cultured cells or animal models). The nucleic acid of interest can be non-integrating or integrating DNA. Integration of DNA into an existing genome could be achieved by co-expression of viral or cellular integration/recombination machinery, and adaption of the DNA cargo to include the corresponding sequences required for integration/recombination.

Cancer therapy is also achievable by expression of a tumor suppressor, death protein, or synthetically lethal gene product. Alternatively, gene expression can be modified via knockdown (e.g., siRNA), or the expression of gene-editing machinery. Cell therapy (genetic modification of cells ex vivo for transfusion post-modification via expression of antibodies, cell receptors, or cell-surface targeting molecules or modification of gene expression (e.g., oncogene) by knockdown (e.g., siRNA), or the expression of gene-editing machinery. Finally, the nanoparticles disclosed herein can be used to advantage for research purposes and scientific investigation.

Table 1 provides a non-exhaustive exemplary list of proteins be encoded by human DNA which can be synthesized and delivered as a therapy for the indicated disease. Preferred target tissue and disease indications are also provided.

TABLE 1

| Encoded | Identifier | | | | SEQ |
|---|---|---|---|---|---|
| Proteins | (UniProt ID) | Disease | Target | Indication | ID NO: |
| Dystrophin | P11532 | Muscular | Muscle | Gene Therapy | 2 |
| | | dystrophy (e.g., | | (gene | |
| | | Duchenne | | replacement) | |
| | | muscular | | | |
| | | dystrophy) | | | |
| Factor VIII | P00451 | Hemophilia A | Liver | Gene Therapy | 3 |
| | | | | (gene | |
| | | | | replacement) | |
| Factor IX | P00740 | Hemophilia B | Liver | Gene Therapy | 4 |
| | | | | (gene | |
| | | | | replacement) | |
| Ornithine | P00480 | Ornithine | Liver | Gene Therapy | 5 |
| transcarbamylase | | transcarbamylase | | (gene | |
| | | deficiency | | replacement) | |
| Alpha-1 | P01009 | Alpha-1 | Liver | Gene Therapy | 6 |
| antitrypsin | | antitrypsin | | (gene | |
| (SERPINA1 gene) | | deficiency | | replacement) | |
| Wilson disease | P35670 | Wilson's disease | Liver | Gene Therapy | 7 |
| protein (WND), | | | | (gene | |
| also known as | | | | replacement) | |
| ATP7B | | | | | |
| LCA2 | Q16518 | Leber congenital | Eye | Gene Therapy | 8 |
| | | amaurosis | | (gene | |
| | | | | replacement) | |
| Survival Motor | Q16637 | Spinal muscular | Spine | Gene Therapy | 9 |
| Neuron (SMN) | | atrophy | | (gene | |
| protein | | | | replacement) | |
| CF | P13569 | Cystic fibrosis | Lung | Gene Therapy | 10 |
| transmembrane | | | | (gene | |
| conductance | | | | replacement) | |
| regulator (CFTR) | | | | | |
| Granulocyte- | P04141 | Cancer (e.g., | Tumor (e.g., | Gene Therapy | 11 |
| macrophage | | melanoma) | melanoma) | (gene | |
| colony- | | | | replacement/anti- | |
| stimulating | | | | cancer) | |
| factor (GM-CSF), | | | | | |
| also known as | | | | | |
| colony- | | | | | |
| stimulating | | | | | |
| factor 2 (CSF2) | | | | | |
| Diphtheria toxin | Q5PY51 | Cancer | Tumor | Gene Therapy | 12 |
| A | | | | (anti-cancer) | |
| Herpes Simplex | Q9QNF7 | Cancer | Tumor | Gene Therapy | 13 |
| Virus Thymidine | | | | (anti-cancer) | |
| Kinase (HSV-tk) | | | | | |
| SARS-CoV-2 Spike | Q202E9 | COVID-19 | Intramuscular | Vaccine | 14 |
| protein | | | | | |
| Influenza A virus | I4EPC4 | Influenza A virus | Intramuscular | Vaccine | 15 |
| (WSN) | | infection | | | |
| hemagglutinin | | | | | |
| (HA) | | | | | |
| Influenza A virus | P09345 | Influenza A virus | Intramuscular | Vaccine | 16 |
| (H5N1) | | infection | | | |
| hemagglutinin | | | | | |
| (HA) | | | | | |

Table 2 lists reagents and targets for more complicated genetic therapies which require coordinated expression or manipulation of different cellular pathways. For example, CAR-T therapy involves removal of a patient's T cells and introducing a gene for a chimeric antigen receptor (CAR) that binds to a surface protein on the patient's cancer cells. CAR T cells are expanded and administered to the patient by infusion. CAR T-cell therapy has shown efficacy for treatment of cancers, particularly blood cancers, and other diseases. For gene editing by Cas9, Cas9 (protein) and a guide RNA (gRNA) are expressed, with the specificity of which gene is removed determined by the sequence of the gRNA. For gene editing by Prime editing, a cas9 (or similar) protein fused to a reverse transcriptase enzyme is expressed along with a prime editing guide RNA (pegRNA), with the specificity of gene editing determined by the sequence of the pegRNA. The prime system introduces single-stranded DNA breaks instead of the double-stranded DNA breaks observed in other editing tools, such as base editors. The components of a base editor complex include a catalytically disabled nuclease fused to a nucleobase deaminase enzyme and, in some cases, a DNA glycosylase inhibitor. RNA base editors achieve analogous changes using components that target RNA. While base editing substitutes a single DNA base with each pass, prime editing allows for the insertion, deletion, and replacement of multiple bases in one pass. Collectively base editing and prime editing offer complementary approaches for making targeted transition mutations.

TABLE 2

| | Identifier | | | | |
|---|---|---|---|---|---|
| Encoded | (e.g., | | | | |
| Proteins/RNA | UniProt ID) | Disease | Target | Indication | Reference cites |
| Chimeric antigen | N/A | Blood cancers | Cancer cells | Gene | Enblad et al., Human |
| receptors (CARs) | | (e.g., B acute | (e.g., B | Therapy (Ex | Gene Therapy. 2015; |
| | | lymphoblastic | lymphocytes) | vivo) | 26(8): 498-505; |
| | | leukemia) | | | US2023/0227551; |
| | | | | | US2023/0227525; |
| | | | | | US2023/0226096; |
| | | | | | U.S. Pat. Nos. |
| | | | | | 9,233,125 and |
| | | | | | 9,624,279; US Patent |
| | | | | | Application |
| | | | | | Publication Nos. |
| | | | | | 20150238631 and |
| | | | | | 20180104354 |
| Cas9 and guide | N/A | Multiple | Multiple | Gene editing | PCT/US2017/047459; |
| RNA (gRNA) | | | | | PCT/US2016/042647; |
| | | | | | U.S. Pat. No. |
| | | | | | 11,624,078 |
| Prime editing | N/A | Multiple | Multiple | Gene editing | PCT/US2020/023712 |
| machinery - | | | | | PCT/US2017/050690 |
| Cas9/reverse | | | | | |
| transcriptase | | | | | |
| fusion protein | | | | | |
| and prime editing | | | | | |
| guide RNA | | | | | |
| (pegRNA) | | | | | |
| Base Editing | N/A | Multiple | Multiple | Gene editing | WO 2020/081568 |
| machinery | | | | | WO 2021/222318 |

Human Dystrophin Amino Acid Sequence (SEQ ID NO: 2):
MLWWEEVEDCYEREDVQKKTFTKWVNAQFSKFGKQHIENLFSDLQDGRRLLDLLEGLTGQKLPKEKGST
RVHALNNVNKALRVLQNNNVDLVNIGSTDIVDGNHKLTLGLIWNIILHWQVKNVMKNIMAGLQQTNSEKI
LLSWVRQSTRNYPQVNVINFTTSWSDGLALNALIHSHRPDLFDWNSVVCQQSATQRLEHAFNIARYQLGIE
KLLDPEDVDTTYPDKKSILMYITSLFQVLPQQVSIEAIQEVEMLPRPPKVTKEEHFQLHHQMHYSQQITVSL
AQGYERTSSPKPRFKSYAYTQAAYVTTSDPTRSPFPSQHLEAPEDKSFGSSLMESEVNLDRYQTALEEVLS
WLLSAEDTLQAQGEISNDVEVVKDQFHTHEGYMMDLTAHQGRVGNILQLGSKLIGTGKLSEDEETEVQEQ
MNLLNSRWECLRVASMEKQSNLHRVLMDLQNQKLKELNDWLTKTEERTRKMEEEPLGPDLEDLKRQVQ
QHKVLQEDLEQEQVRVNSLTHMVVVVDESSGDHATAALEEQLKVLGDRWANICRWTEDRWVLLQDILL
KWQRLTEEQCLFSAWLSEKEDAVNKIHTTGFKDQNEMLSSLQKLAVLKADLEKKKQSMGKLYSLKQDLL
STLKNKSVTQKTEAWLDNFARCWDNLVQKLEKSTAQISQAVTTTQPSLTQTTVMETVTTVTTREQILVKH
AQEELPPPPPQKKRQITVDSEIRKRLDVDITELHSWITRSEAVLQSPEFAIFRKEGNFSDLKEKVNAIEREKAE
KFRKLQDASRSAQALVEQMVNEGVNADSIKQASEQLNSRWIEFCQLLSERLNWLEYQNNIIAFYNQLQQLE
QMTTTAENWLKIQPTTPSEPTAIKSQLKICKDEVNRLSDLQPQIERLKIQSIALKEKGQGPMFLDADFVAFTN
HFKQVFSDVQAREKELQTIFDTLPPMRYQETMSAIRTWVQQSETKLSIPQLSVTDYEIMEQRLGELQALQSS
LQEQQSGLYYLSTTVKEMSKKAPSEISRKYQSEFEEIEGRWKKLSSQLVEHCQKLEEQMNKLRKIQNHIQTL
KKWMAEVDVFLKEEWPALGDSEILKKQLKQCRLLVSDIQTIQPSLNSVNEGGQKIKNEAEPEFASRLETEL
KELNTQWDHMCQQVYARKEALKGGLEKTVSLQKDLSEMHEWMTQAEEEYLERDFEYKTPDELQKAVEE
MKRAKEEAQQKEAKVKLLTESVNSVIAQAPPVAQEALKKELETLTTNYQWLCTRLNGKCKTLEEVWACW
HELLSYLEKANKWLNEVEFKLKTTENIPGGAEEISEVLDSLENLMRHSEDNPNQIRILAQTLTDGGVMDELI
NEELETFNSRWRELHEEAVRRQKLLEQSIQSAQETEKSLHLIQESLTFIDKQLAAYIADKVDAAQMPQEAQ
KIQSDLTSHEISLEEMKKHNQGKEAAQRVLSQIDVAQKKLQDVSMKFRLFQKPANFEQRLQESKMILDEVK
MHLPALETKSVEQEVVQSQLNHCVNLYKSLSEVKSEVEMVIKTGRQIVQKKQTENPKELDERVTALKLHY
NELGAKVTERKQQLEKCLKLSRKMRKEMNVLTEWLAATDMELTKRSAVEGMPSNLDSEVAWGKATQKE
IEKQKVHLKSITEVGEALKTVLGKKETLVEDKLSLLNSNWIAVTSRAEEWLNLLLEYQKHMETFDQNVDHI
TKWIIQADTLLDESEKKKPQQKEDVLKRLKAELNDIRPKVDSTRDQAANLMANRGDHCRKLVEPQISELN
HRFAAISHRIKTGKASIPLKELEQFNSDIQKLLEPLEAEIQQGVNLKEEDFNKDMNEDNEGTVKELLQRGDN
LQQRITDERKREEIKIKQQLLQTKHNALKDLRSQRRKKALEISHQWYQYKRQADDLLKCLDDIEKKLASLP
EPRDERKIKEIDRELQKKKEELNAVRRQAEGLSEDGAAMAVEPTQIQLSKRWREIESKFAQFRRLNFAQIHT
VREETMMVMTEDMPLEISYVPSTYLTEITHVSQALLEVEQLLNAPDLCAKDFEDLFKQEESLKNIKDSLQQS
SGRIDIIHSKKTAALQSATPVERVKLQEALSQLDFQWEKVNKMYKDRQGRFDRSVEKWRRFHYDIKIFNQ
WLTEAEQFLRKTQIPENWEHAKYKWYLKELQDGIGQRQTVVRTLNATGEEIIQQSSKTDASILQEKLGSLN
LRWQEVCKQLSDRKKRLEEQKNILSEFQRDLNEFVLWLEEADNIASIPLEPGKEQQLKEKLEQVKLLVEELP
LRQGILKQLNETGGPVLVSAPISPEEQDKLENKLKQTNLQWIKVSRALPEKQGEIEAQIKDLGQLEKKLEDL
EEQLNHLLLWLSPIRNQLEIYNQPNQEGPFDVKETEIAVQAKQPDVEEILSKGQHLYKEKPATQPVKRKLED
LSSEWKAVNRLLQELRAKQPDLAPGLTTIGASPTQTVTLVTQPVVTKETAISKLEMPSSLMLEVPALADFNR
AWTELTDWLSLLDQVIKSQRVMVGDLEDINEMIIKQKATMQDLEQRRPQLEELITAAQNLKNKTSNQEAR
TIITDRIERIQNQWDEVQEHLQNRRQQLNEMLKDSTQWLEAKEEAEQVLGQARAKLESWKEGPYTVDAIQ
KKITETKQLAKDLRQWQTNVDVANDLALKLLRDYSADDTRKVHMITENINASWRSIHKRVSEREAALEET
HRLLQQFPLDLEKFLAWLTEAETTANVLQDATRKERLLEDSKGVKELMKQWQDLQGEIEAHTDVYHNLD
ENSQKILRSLEGSDDAVLLQRRLDNMNFKWSELRKKSLNIRSHLEASSDQWKRLHLSLQELLVWLQLKDD
ELSRQAPIGGDFPAVQKQNDVHRAFKRELKTKEPVIMSTLETVRIFLTEQPLEGLEKLYQEPRELPPEERAQ
NVTRLLRKQAEEVNTEWEKLNLHSADWQRKIDETLERLQELQEATDELDLKLRQAEVIKGSWQPVGDLLI
DSLQDHLEKVKALRGEIAPLKENVSHVNDLARQLTTLGIQLSPYNLSTLEDLNTRWKLLQVAVEDRVRQL
HEAHRDFGPASQHELSTSVQGPWERAISPNKVPYYINHETQTTCWDHPKMTELYQSLADLNNVRFSAYRT
AMKLRRLQKALCLDLLSLSAACDALDQHNLKQNDQPMDILQIINCLTTIYDRLEQEHNNLVNVPLCVDMC
LNWLLNVYDTGRTGRIRVLSFKTGIISLCKAHLEDKYRYLFKQVASSTGFCDQRRLGLLLHDSIQIPRQLGE
VASFGGSNIEPSVRSCFQFANNKPEIEAALFLDWMRLEPQSMVWLPVLHRVAAAETAKHQAKCNICKECPII
GFRYRSLKHFNYDICQSCFFSGRVAKGHKMHYPMVEYCTPTTSGEDVRDFAKVLKNKFRTKRYFAKHPR
MGYLPVQTVLEGDNMETPVTLINFWPVDSAPASSPQLSHDDTHSRIEHYASRLAEMENSNGSYLNDSISPN
ESIDDEHLLIQHYCQSLNQDSPLSQPRSPAQILISLESEERGELERILADLEEENRNLQAEYDRLKQQHEHKG
LSPLPSPPEMMPTSPQSPRDAELIAEAKLLRQHKGRLEARMQILEDHNKQLESQLHRLRQLLEQPQAEAKV
NGTTVSSPSTSLQRSDSSQPMLLRVVGSQTSDSMGEEDLLSPPQDTSTGLEEVMEQLNNSFPSSRGRNTPGK
PMREDTM
Factor VIII Amino Acid Sequence (SEQ ID NO: 3)
MQIELSTCFFLCLLRFCFSATRRYYLGAVELSWDYMQSDLGELPVDARFPPRVPKSFPFNTSVVYKKTLFVE
FTDHLFNIAKPRPPWMGLLGPTIQAEVYDTVVITLKNMASHPVSLHAVGVSYWKASEGAEYDDQTSQREK
EDDKVFPGGSHTYVWQVLKENGPMASDPLCLTYSYLSHVDLVKDLNSGLIGALLVCREGSLAKEKTQTLH
KFILLFAVFDEGKSWHSETKNSLMQDRDAASARAWPKMHTVNGYVNRSLPGLIGCHRKSVYWHVIGMGT
TPEVHSIFLEGHTFLVRNHRQASLEISPITFLTAQTLLMDLGQFLLFCHISSHQHDGMEAYVKVDSCPEEPQL
RMKNNEEAEDYDDDLTDSEMDVVRFDDDNSPSFIQIRSVAKKHPKTWVHYIAAEEEDWDYAPLVLAPDD
RSYKSQYLNNGPQRIGRKYKKVRFMAYTDETFKTREAIQHESGILGPLLYGEVGDTLLIIFKNQASRPYNIY
PHGITDVRPLYSRRLPKGVKHLKDFPILPGEIFKYKWTVTVEDGPTKSDPRCLTRYYSSFVNMERDLASGLI
GPLLICYKESVDQRGNQIMSDKRNVILFSVFDENRSWYLTENIQRFLPNPAGVQLEDPEFQASNIMHSINGY
VFDSLQLSVCLHEVAYWYILSIGAQTDFLSVFFSGYTFKHKMVYEDTLTLFPFSGETVFMSMENPGLWILG
CHNSDFRNRGMTALLKVSSCDKNTGDYYEDSYEDISAYLLSKNNAIEPRSFSQNSRHPSTRQKQFNATTIPE
NDIEKTDPWFAHRTPMPKIQNVSSSDLLMLLRQSPTPHGLSLSDLQEAKYETFSDDPSPGAIDSNNSLSEMT
HFRPQLHHSGDMVFTPESGLQLRLNEKLGTTAATELKKLDFKVSSTSNNLISTIPSDNLAAGTDNTSSLGPPS
MPVHYDSQLDTTLFGKKSSPLTESGGPLSLSEENNDSKLLESGLMNSQESSWGKNVSSTESGRLFKGKRAH
GPALLTKDNALFKVSISLLKTNKTSNNSATNRKTHIDGPSLLIENSPSVWQNILESDTEFKKVTPLIHDRMLM
DKNATALRLNHMSNKTTSSKNMEMVQQKKEGPIPPDAQNPDMSFFKMLFLPESARWIQRTHGKNSLNSG
QGPSPKQLVSLGPEKSVEGQNFLSEKNKVVVGKGEFTKDVGLKEMVFPSSRNLFLTNLDNLHENNTHNQE
KKIQEEIEKKETLIQENVVLPQIHTVTGTKNFMKNLFLLSTRQNVEGSYDGAYAPVLQDFRSLNDSTNRTKK
HTAHFSKKGEEENLEGLGNQTKQIVEKYACTTRISPNTSQQNFVTQRSKRALKQFRLPLEETELEKRIIVDDT
STQWSKNMKHLTPSTLTQIDYNEKEKGAITQSPLSDCLTRSHSIPQANRSPLPIAKVSSFPSIRPIYLTRVLFQ
DNSSHLPAASYRKKDSGVQESSHFLQGAKKNNLSLAILTLEMTGDQREVGSLGTSATNSVTYKKVENTVLP
KPDLPKTSGKVELLPKVHIYQKDLFPTETSNGSPGHLDLVEGSLLQGTEGAIKWNEANRPGKVPFLRVATES
SAKTPSKLLDPLAWDNHYGTQIPKEEWKSQEKSPEKTAFKKKDTILSLNACESNHAIAAINEGQNKPEIEVT
WAKQGRTERLCSQNPPVLKRHQREITRTTLQSDQEEIDYDDTISVEMKKEDFDIYDEDENQSPRSFQKKTR
HYFIAAVERLWDYGMSSSPHVLRNRAQSGSVPQFKKVVFQEFTDGSFTQPLYRGELNEHLGLLGPYIRAEV
EDNIMVTFRNQASRPYSFYSSLISYEEDQRQGAEPRKNFVKPNETKTYFWKVQHHMAPTKDEFDCKAWAY
FSDVDLEKDVHSGLIGPLLVCHTNTLNPAHGRQVTVQEFALFFTIFDETKSWYFTENMERNCRAPCNIQME
DPTFKENYRFHAINGYIMDTLPGLVMAQDQRIRWYLLSMGSNENIHSIHFSGHVFTVRKKEEYKMALYNL
YPGVFETVEMLPSKAGIWRVECLIGEHLHAGMSTLFLVYSNKCQTPLGMASGHIRDFQITASGQYGQWAP
KLARLHYSGSINAWSTKEPFSWIKVDLLAPMIIHGIKTQGARQKFSSLYISQFIIMYSLDGKKWQTYRGNST
GTLMVFFGNVDSSGIKHNIFNPPIIARYIRLHPTHYSIRSTLRMELMGCDLNSCSMPLGMESKAISDAQITASS
YFTNMFATWSPSKARLHLQGRSNAWRPQVNNPKEWLQVDFQKTMKVTGVTTQGVKSLLTSMYVKEFLIS
SSQDGHQWTLFFQNGKVKVFQGNQDSFTPVVNSLDPPLLTRYLRIHPQSWVHQIALRMEVLGCEAQDLY
Factor IX Amino Acid Sequence (SEQ ID NO: 4):
MQRVNMIMAESPGLITICLLGYLLSAECTVFLDHENANKILNRPKRYNSGKLEEFVQGNLERECMEEKCSF
EEAREVFENTERTTEFWKQYVDGDQCESNPCLNGGSCKDDINSYECWCPFGFEGKNCELDVTCNIKNGRC
EQFCKNSADNKVVCSCTEGYRLAENQKSCEPAVPFPCGRVSVSQTSKLTRAETVFPDVDYVNSTEAETILD
NITQSTQSFNDFTRVVGGEDAKPGQFPWQVVLNGKVDAFCGGSIVNEKWIVTAAHCVETGVKITVVAGEH
NIEETEHTEQKRNVIRIIPHHNYNAAINKYNHDIALLELDEPLVLNSYVTPICIADKEYTNIFLKFGSGYVSGW
GRVFHKGRSALVLQYLRVPLVDRATCLRSTKFTIYNNMFCAGFHEGGRDSCQGDSGGPHVTEVEGTSFLT
GIISWGEECAMKGKYGIYTKVSRYVNWIKEKTKLT
Ornithine transcarbamylase Amino Acid Sequence (SEQ ID NO: 5):
MLFNLRILLNNAAFRNGHNFMVRNFRCGQPLQNKVQLKGRDLLTLKNFTGEEIKYMLWLSADLKFRIKQK
GEYLPLLQGKSLGMIFEKRSTRTRLSTETGFALLGGHPCFLTTQDIHLGVNESLTDTARVLSSMADAVLARV
YKQSDLDTLAKEASIPIINGLSDLYHPIQILADYLTLQEHYSSLKGLTLSWIGDGNNILHSIMMSAAKFGMHL
QAATPKGYEPDASVTKLAEQYAKENGTKLLLTNDPLEAAHGGNVLITDTWISMGQEEEKKKRLQAFQGY
QVTMKTAKVAASDWTFLHCLPRKPEEVDDEVFYSPRSLVFPEAENRKWTIMAVMVSLLTDYSPQLQKPKF
Alpha-1 antitrypsin Amino Acid Sequence (SEQ ID NO: 6):
MPSSVSWGILLLAGLCCLVPVSLAEDPQGDAAQKTDTSHHDQDHPTFNKITPNLAEFAFSLYRQLAHQSNS
TNIFFSPVSIATAFAMLSLGTKADTHDEILEGLNFNLTEIPEAQIHEGFQELLRTLNQPDSQLQLTTGNGLFLS
EGLKLVDKFLEDVKKLYHSEAFTVNFGDTEEAKKQINDYVEKGTQGKIVDLVKELDRDTVFALVNYIFFK
GKWERPFEVKDTEEEDFHVDQVTTVKVPMMKRLGMFNIQHCKKLSSWVLLMKYLGNATAIFFLPDEGKL
QHLENELTHDIITKFLENEDRRSASLHLPKLSITGTYDLKSVLGQLGITKVFSNGADLSGVTEEAPLKLSKAV
HKAVLTIDEKGTEAAGAMFLEAIPMSIPPEVKFNKPFVFLMIEQNTKSPLFMGKVVNPTQK
Wilson disease protein (WND) Amino Acid Sequence (SEQ ID NO: 7):
MPEQERQITAREGASRKILSKLSLPTRAWEPAMKKSFAFDNVGYEGGLDGLGPSSQVATSTVRILGMTCQS
CVKSIEDRISNLKGIISMKVSLEQGSATVKYVPSVVCLQQVCHQIGDMGFEASIAEGKAASWPSRSLPAQEA
VVKLRVEGMTCQSCVSSIEGKVRKLQGVVRVKVSLSNQEAVITYQPYLIQPEDLRDHVNDMGFEAAIKSK
VAPLSLGPIDIERLQSTNPKRPLSSANQNFNNSETLGHQGSHVVTLQLRIDGMHCKSCVLNIEENIGQLLGVQ
SIQVSLENKTAQVKYDPSCTSPVALQRAIEALPPGNFKVSLPDGAEGSGTDHRSSSSHSPGSPPRNQVQGTCS
TTLIAIAGMTCASCVHSIEGMISQLEGVQQISVSLAEGTATVLYNPSVISPEELRAAIEDMGFEASVVSESCST
NPLGNHSAGNSMVQTTDGTPTSVQEVAPHTGRLPANHAPDILAKSPQSTRAVAPQKCFLQIKGMTCASCVS
NIERNLQKEAGVLSVLVALMAGKAEIKYDPEVIQPLEIAQFIQDLGFEAAVMEDYAGSDGNIELTITGMTCA
SCVHNIESKLTRTNGITYASVALATSKALVKFDPEIIGPRDIIKIIEEIGFHASLAQRNPNAHHLDHKMEIKQW
KKSFLCSLVFGIPVMALMIYMLIPSNEPHQSMVLDHNIIPGLSILNLIFFILCTFVQLLGGWYFYVQAYKSLR
HRSANMDVLIVLATSIAYVYSLVILVVAVAEKAERSPVTFFDTPPMLFVFIALGRWLEHLAKSKTSEALAKL
MSLQATEATVVTLGEDNLIIREEQVPMELVQRGDIVKVVPGGKFPVDGKVLEGNTMADESLITGEAMPVT
KKPGSTVIAGSINAHGSVLIKATHVGNDTTLAQIVKLVEEAQMSKAPIQQLADRFSGYFVPFIIIMSTLTLVV
WIVIGFIDFGVVQRYFPNPNKHISQTEVIIRFAFQTSITVLCIACPCSLGLATPTAVMVGTGVAAQNGILIKGG
KPLEMAHKIKTVMFDKTGTITHGVPRVMRVLLLGDVATLPLRKVLAVVGTAEASSEHPLGVAVTKYCKEE
LGTETLGYCTDFQAVPGCGIGCKVSNVEGILAHSERPLSAPASHLNEAGSLPAEKDAVPQTFSVLIGNREWL
RRNGLTISSDVSDAMTDHEMKGQTAILVAIDGVLCGMIAIADAVKQEAALAVHTLQSMGVDVVLITGDNR
KTARAIATQVGINKVFAEVLPSHKVAKVQELQNKGKKVAMVGDGVNDSPALAQADMGVAIGTGTDVAIE
AADVVLIRNDLLDVVASIHLSKRTVRRIRINLVLALIYNLVGIPIAAGVFMPIGIVLQPWMGSAAMAASSVS
VVLSSLQLKCYKKPDLERYEAQAHGHMKPLTASQVSVHIGMDDRWRDSPRATPWDQVSYVSQVSLSSLT
SDKPSRHSAAADDDGDKWSLLLNGRDEEQYI
LCA2 Amino Acid Sequence (SEQ ID NO: 8):
MSIQVEHPAGGYKKLFETVEELSSPLTAHVTGRIPLWLTGSLLRCGPGLFEVGSEPFYHLFDGQALLHKFDF
KEGHVTYHRRFIRTDAYVRAMTEKRIVITEFGTCAFPDPCKNIFSRFFSYFRGVEVTDNALVNVYPVGEDYY
ACTETNFITKINPETLETIKQVDLCNYVSVNGATAHPHIENDGTVYNIGNCFGKNFSIAYNIVKIPPLQADKE
DPISKSEIVVQFPCSDRFKPSYVHSFGLTPNYIVFVETPVKINLFKFLSSWSLWGANYMDCFESNETMGVWL
HIADKKRKKYLNNKYRTSPFNLFHHINTYEDNGFLIVDLCCWKGFEFVYNYLYLANLRENWEEVKKNARK
APQPEVRRYVLPLNIDKADTGKNLVTLPNTTATAILCSDETIWLEPEVLFSGPRQAFEFPQINYQKYCGKPY
TYAYGLGLNHFVPDRLCKLNVKTKETWVWQEPDSYPSEPIFVSHPDALEEDDGVVLSVVVSPGAGQKPAY
LLILNAKDLSEVARAEVEINIPVTFHGLFKKS
Survival Motor Neuron (SMN) Protein Amino Acid Sequence (SEQ ID NO: 9):
MAMSSGGSGGGVPEQEDSVLFRRGTGQSDDSDIWDDTALIKAYDKAVASFKHALKNGDICETSGKPKTTP
KRKPAKKNKSQKKNTAASLQQWKVGDKCSAIWSEDGCIYPATIASIDFKRETCVVVYTGYGNREEQNLSD
LLSPICEVANNIEQNAQENENESQVSTDESENSRSPGNKSDNIKPKSAPWNSFLPPPPPMPGPRLGPGKPGLK
FNGPPPPPPPPPPHLLSCWLPPFPSGPPIIPPPPPICPDSLDDADALGSMLISWYMSGYHTGYYMGFRQNQKEG
RCSHSLN
CF transmembrane conductance regulator (CFTR) Amino Acid Sequence (SEQ ID NO: 10):
MQRSPLEKASVVSKLFFSWTRPILRKGYRQRLELSDIYQIPSVDSADNLSEKLEREWDRELASKKNPKLINA
LRRCFFWRFMFYGIFLYLGEVTKAVQPLLLGRIIASYDPDNKEERSIAIYLGIGLCLLFIVRTLLLHPAIFGLH
HIGMQMRIAMFSLIYKKTLKLSSRVLDKISIGQLVSLLSNNLNKFDEGLALAHFVWIAPLQVALLMGLIWEL
LQASAFCGLGFLIVLALFQAGLGRMMMKYRDQRAGKISERLVITSEMIENIQSVKAYCWEEAMEKMIENL
RQTELKLTRKAAYVRYFNSSAFFFSGFFVVFLSVLPYALIKGIILRKIFTTISFCIVLRMAVTRQFPWAVQTW
YDSLGAINKIQDFLQKQEYKTLEYNLTTTEVVMENVTAFWEEGFGELFEKAKQNNNNRKTSNGDDSLFFS
NFSLLGTPVLKDINFKIERGQLLAVAGSTGAGKTSLLMVIMGELEPSEGKIKHSGRISFCSQFSWIMPGTIKE
NIIFGVSYDEYRYRSVIKACQLEEDISKFAEKDNIVLGEGGITLSGGQRARISLARAVYKDADLYLLDSPFGY
LDVLTEKEIFESCVCKLMANKTRILVTSKMEHLKKADKILILHEGSSYFYGTFSELQNLQPDFSSKLMGCDS
FDQFSAERRNSILTETLHRFSLEGDAPVSWTETKKQSFKQTGEFGEKRKNSILNPINSIRKFSIVQKTPLQMN
GIEEDSDEPLERRLSLVPDSEQGEAILPRISVISTGPTLQARRRQSVLNLMTHSVNQGQNIHRKTTASTRKVS
LAPQANLTELDIYSRRLSQETGLEISEEINEEDLKECFFDDMESIPAVTTWNTYLRYITVHKSLIFVLIWCLVIF
LAEVAASLVVLWLLGNTPLQDKGNSTHSRNNSYAVIITSTSSYYVFYIYVGVADTLLAMGFFRGLPLVHTLI
TVSKILHHKMLHSVLQAPMSTLNTLKAGGILNRFSKDIAILDDLLPLTIFDFIQLLLIVIGAIAVVAVLQPYIFV
ATVPVIVAFIMLRAYFLQTSQQLKQLESEGRSPIFTHLVTSLKGLWTLRAFGRQPYFETLFHKALNLHTANW
FLYLSTLRWFQMRIEMIFVIFFIAVTFISILTTGEGEGRVGIILTLAMNIMSTLQWAVNSSIDVDSLMRSVSRV
FKFIDMPTEGKPTKSTKPYKNGQLSKVMIIENSHVKKDDIWPSGGQMTVKDLTAKYTEGGNAILENISFSISP
GQRVGLLGRTGSGKSTLLSAFLRLLNTEGEIQIDGVSWDSITLQQWRKAFGVIPQKVFIFSGTFRKNLDPYE
QWSDQEIWKVADEVGLRSVIEQFPGKLDFVLVDGGCVLSHGHKQLMCLARSVLSKAKILLLDEPSAHLDP
VTYQIIRRTLKQAFADCTVILCEHRIEAMLECQQFLVIEENKVRQYDSIQKLLNERSLFRQAISPSDRVKLFPH
RNSSKCKSKPQIAALKEETEEEVQDTRL
Granulocyte-macrophage colony-stimulating factor (GM-CSF), Amino Acid Sequence (SEQ
ID NO: 11):
MWLQSLLLLGTVACSISAPARSPSPSTQPWEHVNAIQEARRLLNLSRDTAAEMNETVEVISEMFDLQEPTCL
QTRLELYKQGLRGSLTKLKGPLTMMASHYKQHCPPTPETSCATQIITFESFKENLKDFLLVIPFDCWEPVQE
Diphtheria toxin A, Amino Acid Sequence (SEQ ID NO: 12):
MGADDVVDSSKSFVMENFSSYHGTKPGYVDSIQKGIQKPKSGTQGNYDDDWKGFYSTDNKYDAAGYSV
DNENPLSGKAGGVVKVTYPGLTKVLALKVDNAETIKKELGLSLTEPLMEQVGTEEFIKRFGDGASRVVLSL
PFAEGSSSVEYINNWEQAKALSVELEINFETRGKRGQDAMYEYMAQACAGNRVRRSVGSSLSCINLDWDV
IRDKTKTKIESLKEHGPIKNKMSESPNKTVSEEKAKQYLEEFHQTALEHPELSELKTVTGTNPVFAGANYAA
WAVNVAQVIDSETADNLEKTTAALSILPGIGSVMGIADGAVHHNTEEIVAQSIALSSLMVAQAIPLVGELVD
IGFAAYNFVESIINLFQVVHNSYNRPAYSPGHKTQPFLHDGYAVSWNTVEDSIIRTGFQGESGHDIKITAENT
PLPIAGVLLPTIPGKLDVNKSKTHISVNGRKIRMRCRAIDGDVTFCRPKSPVYVGNGVHANLHVAFHRSSSE
KIHSNEISSDSIGVLGYQKTVDHTKVNSKLSLFFEIKS
Herpes Simplex Virus Thymidine Kinase (HSV-tk) (SEQ ID NO: 13):
MASYPCHQHASAFDQAARSRGHSNRRTALRPRRQQEATEVRLEQKMPTLLRVYIDGPHGMGKTTTTQLL
VALGSRDDIVYVPEPMTYWQVLGASETIANIYTTQHRLDQGEISAGDAAVVMTSAQITMGMPYAVTDAVL
APHIGGEAGSSHAPPPALTLIFDRHPIAALLCYPAARYLMGSMTPQAVLAFVALIPPTLPGTNIVLGALPEDR
HIDRLAKRQRPGERLDLAMLAAIRRVYGLLANTVRYLQGGGSWREDWGQLSGTAVPPQGAEPQSNAGPR
PHIGDTLFTLFRAPELLAPNGDLYNVFAWALDVLAKRLRPMHVFILDYDQSPAGCRDALLQLTSGMVQTH
VTTPGSIPTICDLARTFAREMGEAN
SARS-CoV-2 Spike protein (SEQ ID NO: 14):
MFIFLLFLTLTSGSDLDRCTTFDDVQAPNYTQHTSSMRGVYYPDEIFRSDTLYLTQDLFLPFYSNVTGFHTIN
HTFGNPVIPFKDGIYFAATEKSNVVRGWVFGSTMNNKSQSVIIINNSTNVVIRACNFELCDNPFFAVSKPMG
TQTHTMIFDNAFNCTFEYISDAFSLDVSEKSGNFKHLREFVFKNKDGFLYVYKGYQPIDVVRDLPSGFNTLK
PIFKLPLGINITNFRAILTAFSPAQDIWGTSAAAYFVGYLKPTTFMLKYDENGTITDAVDCSQNPLAELKCSV
KSFEIDKGIYQTSNFRVVPSGDVVRFPNITNLCPFGEVFNATKFPSVYAWERKKISNCVADYSVLYNSTFFST
FKCYGVSATKLNDLCFSNVYADSFVVKGDDVRQIAPGQTGVIADYNYKLPDDFMGCVLAWNTRNIDATS
TGNYNYKYRYLRHGKLRPFERDISNVPFSPDGKPCTPPALNCYWPLNDYGFYTTTGIGYQPYRVVVLSFEL
LNAPATVCGPKLSTDLIKNQCVNFNFNGLTGTGVLTPSSKRFQPFQQFGRDVSDFTDSVRDPKTSEILDISPC
SFGGVSVITPGTNASSEVAVLYQDVNCTDVSTAIHADQLTPAWRIYSTGNNVFQTQAGCLIGAEHVDTSYE
CDIPIGAGICASYHTVSLLRSTSQKSIVAYTMSLGADSSIAYSNNTIAIPTNFSISITTEVMPVSMAKTSVDCN
MYICGDSTECANLLLQYGSFCTQLNRALSGIAAEQDRNTREVFAQVKQMYKTPTLKYFGGFNFSQILPDPL
KPTKRSFIEDLLFNKVTLADAGFMKQYGECLGDINARDLICAQKFNGLTVLPPLLTDDMIAAYTAALVSGT
ATAGWTFGAGAALQIPFAMQMAYRFNGIGVTQNVLYENQKQIANQFNKAISQIQESLTTTSTALGKLQDV
VNQNAQALNTLVKQLSSNFGAISSVLNDILSRLDKVEAEVQIDRLITGRLQSLQTYVTQQLIRAAEIRASANL
AATKMSECVLGQSKRVDFCGKGYHLMSFPQAAPHGVVFLHVTYVPSQERNFTTAPAICHEGKAYFPREGV
FVFNGTSWFITQRNFFSPQIITTDNTFVSGNCDVVIGIINNTVYDPLQPELDSFKEELDKYFKNHTSPDVDLG
DISGINASVVNIQKEIDRLNEVAKNLNESLIDLQELGKYEQYIKWPWYVWLGFIAGLIAIVMVTILLCCMTS
CCSCLKGACSCGSCCKFDEDDSEPVLKGVKLHYT
Influenza A virus (WSN) hemagglutinin (HA) (SEQ ID NO. 15)
MKAFVLVLLYAFVATDADTICIGYHANNSTDTVDTIFEKNVAVTHSVNLLEDRHNGKLCKLKGIAPLQLG
KCNITGWLLGNPECDSLLPARSWSYIVETPNSENGACYPGDFIDYEELREQLSSVSSLERFEIFPKESSWPNH
TFNGVTVSCSHRGKSSFYRNLLWLTKKGDSYPKLTNSYVNNKGKEVLVLWGVHHPSSSDEQQSLYSNGN
AYVSVASSNYNRRFTPEIAARPKVKDQHGRMNYYWTLLEPGDTIIFEATGNLIAPWYAFALSRGFESGIITS
NASMHECNTKCQTPQGSINSNLPFQNIHPVTIGECPKYVRSTKLRMVTGLRNIPSIQYRGLFGAIAGFIEGGW
TGMIDGWYGYHHQNEQGSGYAADQKSTQNAINRITNKVNSVIEKMNTQFTAVGKEFNNLEKRMENLNKK
VDDGFLDIWTYNAELLVLLENERTLDFHDLNVKNLYEKVKSQLKNNAKEIGNGCFEFYHKCDNECMESVR
NGTYDYPKYSEESKLNREKIDGVKLESMGVYQILAIYSTVASSLVLLVSLGAISFWMCSNGSLQCRICI
Influenza A virus (H5N1) hemagglutinin (HA) (SEQ ID NO: 16)
MERIVLLLAIVSLVKSDQICIGYHANKSTKQVDTIMEKNVTVTHAQDILERTHNGKLCSLNGVKPLILRDCS
VAGWLLGNPMCDEFLNVPEWSYIVEKDNPINSLCYPGDFNDYEELKHLLSSTNHFEKIQIIPRSSWSNHDAS
SGVSSACPYIGRSSFFRNVVWLIKKDNAYPTIKRSYNNTNQEDLLILWGIHHPNDAAEQTKLYQNPTTYVSV
GTSTLNQRSIPEIATRPKVNGQSGRMEFFWTILKPNDAINFESNGNFIAPEYAYKIVKKGDSAIMKSGLAYG
NCDTKCQTPVGAINSSMPFHNIHPHTIGECPKYVKSDRLVLATGLRNVPQRKKRGLFGAIAGFIEGGWQGM
VDGWYGYHHSNEQGSGYAADKESTQKAIDGITNKVNSIIDKMNTQFKAVGKEFNNLERRVENLNKKMED
GFLDVWTYNVELLVLMENERTLDFHDSNVKNLYDKVRLQLKDNARELGNGCFEFYHKCDNECMESVRN
GTYDYPQYSEEARLNREEISGVKLESMGVYQILSIYSTVASSLALAIMIAGLSFWMCSNGSLQCRICI
52K protein (UniProt ID NO: P04496) (SEQ ID NO: 17)
MHPVLRQMRPPPQQRQEQEQRQTCRAPSPPPTASGGATSAVDAAADGDYEPPRRRARHYLDLEEGEGLAR
LGAPSPERYPRVQLKRDTREAYVPRQNLFRDREGEEPEEMRDRKFHAGRELRHGLNRERLLREEDFEPDAR
TGISPARAHVAAADLVTAYEQTVNQEINFQKSENNHVRTLVAREEVAIGLMHLWDFVSALEQNPNSKPLM
AQLFLIVQHSRDNEAFRDALLNIVEPEGRWLLDLINILQSIVVQERSLSLADKVAAINYSMLSLGKFYARKIY
HTPYVPIDKEVKIEGFYMRMALKVLTLSDDLGVYRNERIHKAVSVSRRRELSDRELMHSLQRALAGTGSG
DREAESYFDAGADLRWAPSRRALEAAGAGPGLAVAPARAGNVGGVEEYDEDDEYEPEDGEY
52K protein (UniProt ID NO: Q6VGV2) (SEQ ID NO: 18)
MHPVLRQMRPPPQQRQEQEQRQTCRAPSPPPTASGGATSAVDAAADGDYEPPRRRARHYLDLEEGEGLAR
LGAPSPERHPRVQLKRDTREAYVPRQNLFRDREGEEPEEMRDRKFHAGRELRHGLNRERLLREEDFEPDAR
TGISPARAHVAAADLVTAYEQTVNQEINFQKSFNNHVRTLVAREEVAIGLMHLWDFVSALEQNPNSKPLM
AQLFLIVQHSRDNEAFRDALLNIVEPEGRWLLDLINILQSIVVQERSLSLADKVAAINYSMLSLGKFYARKIY
HTPYVPIDKEVKIEGFYMRMALKVLTLSDDLGVYRNERIHKAVSVSRRRELSDRELMHSLQRALAGTGSG
DREAESYFDAGADLRWAPSRRALEAAGAGPGLAVAPARAGNVGGVEEYDEDDEYEPEDGEY

While certain features of the invention have been described herein, many modifications, substitutions, changes, and equivalents will now occur to those of ordinary skill in the art. It is, therefore, to be understood that the appended claims are intended to cover all such modifications and changes as fall within the true spirit of the invention.

1. A nanoparticle formulation for delivery of one or more heterologous molecules of interest to a cell, comprising a self-assembling p52K protein which forms a heterologous molecule-p52K complex, in a pharmaceutically acceptable carrier.

2. The nanoparticle formulation of claim 1, wherein said p52K protein is encoded by a nucleic acid and comprises at least one ITR sequence at the 5′end, the 3′ end or both.

3. The nanoparticle formulation of claim 1, wherein said ITR region is present at
a) both the 5′ and 3′ ends of the p52K the nucleic acid of interest, or
b) the 5′end of the nucleic acid of interest.

4. (canceled)

5. The nanoparticle formulation of claim 1, wherein said 52K protein comprises an IDR region at the N terminus, the C terminus or both, said IDR facilitating self-assembly of said nucleic acid-p52 complex.

6. The nanoparticle formulation of claim 1, further comprising protein VII or one or more polypeptides.

7. (canceled)

8. The nanoparticle formulation of claim 1, which is encapsulated in a liposome or micelle.

9. The nanoparticle formulation of claim 8, wherein said liposome or micelle further comprises targeting ligands on the surface thereof.

10. The nanoparticle formulation of claim 8 wherein the liposome or micelle comprises a hydrophobic group selected from the group consisting of lipophilic alkyl groups, cholesterol, and combinations thereof.

11. The nanoparticle formulation of claim 1, wherein said nucleic acid of interest encodes a virus genome or a metabolic gene.

12. (canceled)

13. The nanoparticle formulation of claim 1, wherein said nucleic acid of interest encodes an inhibitory RNA molecule selected from an siRNA, an shRNA or a ribozyme.

14. The nanoparticle formulation of claim 1, wherein said at least one heterologous molecule includes a nucleic acid of interest encoding a guide RNA suitable for use with CRISPR Cas and a Cas9 enzyme.

15. The nanoparticle formulation of claim 1, wherein said nucleic acid of interest is modified to limit recognition by an innate immune response.

16. The nanoparticle formulation of claim 1, wherein said nucleic acid of interest expresses a chimeric antigen receptor.

17. The nanoparticle formulation of claim 1, wherein said at least one heterologous molecule includes a nucleic acid of interest said guide RNA targets a site for single base editing and an enzyme suitable for use in base editing.

18. (canceled)

19. The nanoparticle formulation of claim 1, wherein said at least one heterologous molecules is a nucleic acid encoding a protein which upon administration to a subject in need thereof, ameliorates symptoms for an indicated disorder listed in Table 2.

20. A kit comprising a nanoparticle formulation of claim 1 and instructions for use thereof.

21. A method of preparing a nanoparticle formulation of claim 1, the method comprising: contacting a p52K protein or functional fragment thereof with at least one heterologous molecule of interest under conditions wherein a p52K protein-heterologous molecule containing complex forms, said complex being in a solution suitable for administration to a cell type of interest.

22. The method of claim 21 for preparation of a nanoparticle formulation comprising at least one heterologous molecule of interest selected from a nucleic acid of interest which encodes a virus genome, a nucleic acid of interest which encodes a metabolic gene, a nucleic acid of interest encoding an inhibitory RNA, an siRNA, an shRNA and a ribozyme, a nucleic acid of interest encoding a guide RNA suitable for use with CRISPR Cas and a Cas9 enzyme protein, a nucleic acid of interest is modified to limit recognition by an innate immune response, a nucleic acid of interest expresses a chimeric antigen receptor and a nucleic acid of interest said guide RNA targets a site for single base editing and an enzyme suitable for use in base editing.

23. A method for delivery of at least one heterologous molecule of interest to a patient in need thereof, the method comprising administering an effective amount of the nanoparticle formulation of claim 1.

24. A kit for practice of the methods of claim 21, comprising a self assembling p52K protein nanoparticle formulation, at least one heterologous molecule of interest and instructions for use.


SEQUENCE LISTING (18 sequences)

SEQ ID NO: 1 - protein, 22 aa, synthetic construct
  SITE 12..15: note = PEG5000
AKTLLPTPGGSXXXXKKKDOTA

SEQ ID NO: 2 - protein, 3685 aa, Homo sapiens
MLWWEEVEDCYEREDVQKKTFTKWVNAQFSKFGKQHIENLFSDLQDGRRLLDLLEGLTGQKLPKEKGSTRVHALNNVNKALRVLQNNNVDLVNIGSTDIVDGNHKLTLGLIWNIILHWQVKNVMKNIMAGLQQTNSEKILLSWVRQSTRNYPQVNVINFTTSWSDGLALNALIHSHRPDLFDWNSVVCQQSATQRLEHAFNIARYQLGIEKLLDPEDVDTTYPDKKSILMYITSLFQVLPQQVSIEAIQEVEMLPRPPKVTKEEHFQLHHQMHYSQQITVSLAQGYERTSSPKPRFKSYAYTQAAYVTTSDPTRSPFPSQHLEAPEDKSFGSSLMESEVNLDRYQTALEEVLSWLLSAEDTLQAQGEISNDVEVVKDQFHTHEGYMMDLTAHQGRVGNILQLGSKLIGTGKLSEDEETEVQEQMNLLNSRWECLRVASMEKQSNLHRVLMDLQNQKLKELNDWLTKTEERTRKMEEEPLGPDLEDLKRQVQQHKVLQEDLEQEQVRVNSLTHMVVVVDESSGDHATAALEEQLKVLGDRWANICRWTEDRWVLLQDILLKWQRLTEEQCLFSAWLSEKEDAVNKIHTTGFKDQNEMLSSLQKLAVLKADLEKKKQSMGKLYSLKQDLLSTLKNKSVTQKTEAWLDNFARCWDNLVQKLEKSTAQISQAVTTTQPSLTQTTVMETVTTVTTREQILVKHAQEELPPPPPQKKRQITVDSEIRKRLDVDITELHSWITRSEAVLQSPEFAIFRKEGNFSDLKEKVNAIEREKAEKFRKLQDASRSAQALVEQMVNEGVNADSIKQASEQLNSRWIEFCQLLSERLNWLEYQNNIIAFYNQLQQLEQMTTTAENWLKIQPTTPSEPTAIKSQLKICKDEVNRLSDLQPQIERLKIQSIALKEKGQGPMFLDADFVAFTNHFKQVFSDVQAREKELQTIFDTLPPMRYQETMSAIRTWVQQSETKLSIPQLSVTDYEIMEQRLGELQALQSSLQEQQSGLYYLSTTVKEMSKKAPSEISRKYQSEFEEIEGRWKKLSSQLVEHCQKLEEQMNKLRKIQNHIQTLKKWMAEVDVFLKEEWPALGDSEILKKQLKQCRLLVSDIQTIQPSLNSVNEGGQKIKNEAEPEFASRLETELKELNTQWDHMCQQVYARKEALKGGLEKTVSLQKDLSEMHEWMTQAEEEYLERDFEYKTPDELQKAVEEMKRAKEEAQQKEAKVKLLTESVNSVIAQAPPVAQEALKKELETLTTNYQWLCTRLNGKCKTLEEVWACWHELLSYLEKANKWLNEVEFKLKTTENIPGGAEEISEVLDSLENLMRHSEDNPNQIRILAQTLTDGGVMDELINEELETFNSRWRELHEEAVRRQKLLEQSIQSAQETEKSLHLIQESLTFIDKQLAAYIADKVDAAQMPQEAQKIQSDLTSHEISLEEMKKHNQGKEAAQRVLSQIDVAQKKLQDVSMKFRLFQKPANFEQRLQESKMILDEVKMHLPALETKSVEQEVVQSQLNHCVNLYKSLSEVKSEVEMVIKTGRQIVQKKQTENPKELDERVTALKLHYNELGAKVTERKQQLEKCLKLSRKMRKEMNVLTEWLAATDMELTKRSAVEGMPSNLDSEVAWGKATQKEIEKQKVHLKSITEVGEALKTVLGKKETLVEDKLSLLNSNWIAVTSRAEEWLNLLLEYQKHMETFDQNVDHITKWIIQADTLLDESEKKKPQQKEDVLKRLKAELNDIRPKVDSTRDQAANLMANRGDHCRKLVEPQISELNHRFAAISHRIKTGKASIPLKELEQFNSDIQKLLEPLEAEIQQGVNLKEEDFNKDMNEDNEGTVKELLQRGDNLQQRITDERKREEIKIKQQLLQTKHNALKDLRSQRRKKALEISHQWYQYKRQADDLLKCLDDIEKKLASLPEPRDERKIKEIDRELQKKKEELNAVRRQAEGLSEDGAAMAVEPTQIQLSKRWREIESKFAQFRRLNFAQIHTVREETMMVMTEDMPLEISYVPSTYLTEITHVSQALLEVEQLLNAPDLCAKDFEDLFKQEESLKNIKDSLQQSSGRIDIIHSKKTAALQSATPVERVKLQEALSQLDFQWEKVNKMYKDRQGRFDRSVEKWRRFHYDIKIFNQWLTEAEQFLRKTQIPENWEHAKYKWYLKELQDGIGQRQTVVRTLNATGEEIIQQSSKTDASILQEKLGSLNLRWQEVCKQLSDRKKRLEEQKNILSEFQRDLNEFVLWLEEADNIASIPLEPGKEQQLKEKLEQVKLLVEELPLRQGILKQLNETGGPVLVSAPISPEEQDKLENKLKQTNLQWIKVSRALPEKQGEIEAQIKDLGQLEKKLEDLEEQLNHLLLWLSPIRNQLEIYNQPNQEGPFDVKETEIAVQAKQPDVEEILSKGQHLYKEKPATQPVKRKLEDLSSEWKAVNRLLQELRAKQPDLAPGLTTIGASPTQTVTLVTQPVVTKETAISKLEMPSSLMLEVPALADFNRAWTELTDWLSLLDQVIKSQRVMVGDLEDINEMIIKQKATMQDLEQRRPQLEELITAAQNLKNKTSNQEARTIITDRIERIQNQWDEVQEHLQNRRQQLNEMLKDSTQWLEAKEEAEQVLGQARAKLESWKEGPYTVDAIQKKITETKQLAKDLRQWQTNVDVANDLALKLLRDYSADDTRKVHMITENINASWRSIHKRVSEREAALEETHRLLQQFPLDLEKFLAWLTEAETTANVLQDATRKERLLEDSKGVKELMKQWQDLQGEIEAHTDVYHNLDENSQKILRSLEGSDDAVLLQRRLDNMNFKWSELRKKSLNIRSHLEASSDQWKRLHLSLQELLVWLQLKDDELSRQAPIGGDFPAVQKQNDVHRAFKRELKTKEPVIMSTLETVRIFLTEQPLEGLEKLYQEPRELPPEERAQNVTRLLRKQAEEVNTEWEKLNLHSADWQRKIDETLERLQELQEATDELDLKLRQAEVIKGSWQPVGDLLIDSLQDHLEKVKALRGEIAPLKENVSHVNDLARQLTTLGIQLSPYNLSTLEDLNTRWKLLQVAVEDRVRQLHEAHRDFGPASQHFLSTSVQGPWERAISPNKVPYYINHETQTTCWDHPKMTELYQSLADLNNVRFSAYRTAMKLRRLQKALCLDLLSLSAACDALDQHNLKQNDQPMDILQIINCLTTIYDRLEQEHNNLVNVPLCVDMCLNWLLNVYDTGRTGRIRVLSFKTGIISLCKAHLEDKYRYLFKQVASSTGFCDQRRLGLLLHDSIQIPRQLGEVASFGGSNIEPSVRSCFQFANNKPEIEAALFLDWMRLEPQSMVWLPVLHRVAAAETAKHQAKCNICKECPIIGFRYRSLKHFNYDICQSCFFSGRVAKGHKMHYPMVEYCTPTTSGEDVRDFAKVLKNKFRTKRYFAKHPRMGYLPVQTVLEGDNMETPVTLINFWPVDSAPASSPQLSHDDTHSRIEHYASRLAEMENSNGSYLNDSISPNESIDDEHLLIQHYCQSLNQDSPLSQPRSPAQILISLESEERGELERILADLEEENRNLQAEYDRLKQQHEHKGLSPLPSPPEMMPTSPQSPRDAELIAEAKLLRQHKGRLEARMQILEDHNKQLESQLHRLRQLLEQPQAEAKVNGTTVSSPSTSLQRSDSSQPMLLRVVGSQTSDSMGEEDLLSPPQDTSTGLEEVMEQLNNSFPSSRGRNTPGKPMREDTM

SEQ ID NO: 3 - protein, 2351 aa, Homo sapiens
MQIELSTCFFLCLLRFCFSATRRYYLGAVELSWDYMQSDLGELPVDARFPPRVPKSFPFNTSVVYKKTLFVEFTDHLFNIAKPRPPWMGLLGPTIQAEVYDTVVITLKNMASHPVSLHAVGVSYWKASEGAEYDDQTSQREKEDDKVFPGGSHTYVWQVLKENGPMASDPLCLTYSYLSHVDLVKDLNSGLIGALLVCREGSLAKEKTQTLHKFILLFAVFDEGKSWHSETKNSLMQDRDAASARAWPKMHTVNGYVNRSLPGLIGCHRKSVYWHVIGMGTTPEVHSIFLEGHTFLVRNHRQASLEISPITFLTAQTLLMDLGQFLLFCHISSHQHDGMEAYVKVDSCPEEPQLRMKNNEEAEDYDDDLTDSEMDVVRFDDDNSPSFIQIRSVAKKHPKTWVHYIAAEEEDWDYAPLVLAPDDRSYKSQYLNNGPQRIGRKYKKVRFMAYTDETFKTREAIQHESGILGPLLYGEVGDTLLIIFKNQASRPYNIYPHGITDVRPLYSRRLPKGVKHLKDFPILPGEIFKYKWTVTVEDGPTKSDPRCLTRYYSSFVNMERDLASGLIGPLLICYKESVDQRGNQIMSDKRNVILFSVFDENRSWYLTENIQRFLPNPAGVQLEDPEFQASNIMHSINGYVFDSLQLSVCLHEVAYWYILSIGAQTDFLSVFFSGYTFKHKMVYEDTLTLFPFSGETVFMSMENPGLWILGCHNSDFRNRGMTALLKVSSCDKNTGDYYEDSYEDISAYLLSKNNAIEPRSFSQNSRHPSTRQKQFNATTIPENDIEKTDPWFAHRTPMPKIQNVSSSDLLMLLRQSPTPHGLSLSDLQEAKYETFSDDPSPGAIDSNNSLSEMTHFRPQLHHSGDMVFTPESGLQLRLNEKLGTTAATELKKLDFKVSSTSNNLISTIPSDNLAAGTDNTSSLGPPSMPVHYDSQLDTTLFGKKSSPLTESGGPLSLSEENNDSKLLESGLMNSQESSWGKNVSSTESGRLFKGKRAHGPALLTKDNALFKVSISLLKTNKTSNNSATNRKTHIDGPSLLIENSPSVWQNILESDTEFKKVTPLIHDRMLMDKNATALRLNHMSNKTTSSKNMEMVQQKKEGPIPPDAQNPDMSFFKMLFLPESARWIQRTHGKNSLNSGQGPSPKQLVSLGPEKSVEGQNFLSEKNKVVVGKGEFTKDVGLKEMVFPSSRNLFLTNLDNLHENNTHNQEKKIQEEIEKKETLIQENVVLPQIHTVTGTKNFMKNLFLLSTRQNVEGSYDGAYAPVLQDFRSLNDSTNRTKKHTAHFSKKGEEENLEGLGNQTKQIVEKYACTTRISPNTSQQNFVTQRSKRALKQFRLPLEETELEKRIIVDDTSTQWSKNMKHLTPSTLTQIDYNEKEKGAITQSPLSDCLTRSHSIPQANRSPLPIAKVSSFPSIRPIYLTRVLFQDNSSHLPAASYRKKDSGVQESSHFLQGAKKNNLSLAILTLEMTGDQREVGSLGTSATNSVTYKKVENTVLPKPDLPKTSGKVELLPKVHIYQKDLFPTETSNGSPGHLDLVEGSLLQGTEGAIKWNEANRPGKVPFLRVATESSAKTPSKLLDPLAWDNHYGTQIPKEEWKSQEKSPEKTAFKKKDTILSLNACESNHAIAAINEGQNKPEIEVTWAKQGRTERLCSQNPPVLKRHQREITRTTLQSDQEEIDYDDTISVEMKKEDFDIYDEDENQSPRSFQKKTRHYFIAAVERLWDYGMSSSPHVLRNRAQSGSVPQFKKVVFQEFTDGSFTQPLYRGELNEHLGLLGPYIRAEVEDNIMVTFRNQASRPYSFYSSLISYEEDQRQGAEPRKNFVKPNETKTYFWKVQHHMAPTKDEFDCKAWAYFSDVDLEKDVHSGLIGPLLVCHTNTLNPAHGRQVTVQEFALFFTIFDETKSWYFTENMERNCRAPCNIQMEDPTFKENYRFHAINGYIMDTLPGLVMAQDQRIRWYLLSMGSNENIHSIHFSGHVFTVRKKEEYKMALYNLYPGVFETVEMLPSKAGIWRVECLIGEHLHAGMSTLFLVYSNKCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGCDLNSCSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGCEAQDLY

SEQ ID NO: 4 - protein, 461 aa, Homo sapiens
MQRVNMIMAESPGLITICLLGYLLSAECTVFLDHENANKILNRPKRYNSGKLEEFVQGNLERECMEEKCSFEEAREVFENTERTTEFWKQYVDGDQCESNPCLNGGSCKDDINSYECWCPFGFEGKNCELDVTCNIKNGRCEQFCKNSADNKVVCSCTEGYRLAENQKSCEPAVPFPCGRVSVSQTSKLTRAETVFPDVDYVNSTEAETILDNITQSTQSFNDFTRVVGGEDAKPGQFPWQVVLNGKVDAFCGGSIVNEKWIVTAAHCVETGVKITVVAGEHNIEETEHTEQKRNVIRIIPHHNYNAAINKYNHDIALLELDEPLVLNSYVTPICIADKEYTNIFLKFGSGYVSGWGRVFHKGRSALVLQYLRVPLVDRATCLRSTKFTIYNNMFCAGFHEGGRDSCQGDSGGPHVTEVEGTSFLTGIISWGEECAMKGKYGIYTKVSRYVNWIKEKTKLT

SEQ ID NO: 5 - protein, 354 aa, Homo sapiens
MLFNLRILLNNAAFRNGHNFMVRNFRCGQPLQNKVQLKGRDLLTLKNFTGEEIKYMLWLSADLKFRIKQKGEYLPLLQGKSLGMIFEKRSTRTRLSTETGFALLGGHPCFLTTQDIHLGVNESLTDTARVLSSMADAVLARVYKQSDLDTLAKEASIPIINGLSDLYHPIQILADYLTLQEHYSSLKGLTLSWIGDGNNILHSIMMSAAKFGMHLQAATPKGYEPDASVTKLAEQYAKENGTKLLLTNDPLEAAHGGNVLITDTWISMGQEEEKKKRLQAFQGYQVTMKTAKVAASDWTFLHCLPRKPEEVDDEVFYSPRSLVFPEAENRKWTIMAVMVSLLTDYSPQLQKPKF

SEQ ID NO: 6 - protein, 418 aa, Homo sapiens
MPSSVSWGILLLAGLCCLVPVSLAEDPQGDAAQKTDTSHHDQDHPTFNKITPNLAEFAFSLYRQLAHQSNSTNIFFSPVSIATAFAMLSLGTKADTHDEILEGLNFNLTEIPEAQIHEGFQELLRTLNQPDSQLQLTTGNGLFLSEGLKLVDKFLEDVKKLYHSEAFTVNFGDTEEAKKQINDYVEKGTQGKIVDLVKELDRDTVFALVNYIFFKGKWERPFEVKDTEEEDFHVDQVTTVKVPMMKRLGMFNIQHCKKLSSWVLLMKYLGNATAIFFLPDEGKLQHLENELTHDIITKFLENEDRRSASLHLPKLSITGTYDLKSVLGQLGITKVFSNGADLSGVTEEAPLKLSKAVHKAVLTIDEKGTEAAGAMFLEAIPMSIPPEVKFNKPFVFLMIEQNTKSPLFMGKVVNPTQK

SEQ ID NO: 7 - protein, 1465 aa, Homo sapiens
MPEQERQITAREGASRKILSKLSLPTRAWEPAMKKSFAFDNVGYEGGLDGLGPSSQVATSTVRILGMTCQSCVKSIEDRISNLKGIISMKVSLEQGSATVKYVPSVVCLQQVCHQIGDMGFEASIAEGKAASWPSRSLPAQEAVVKLRVEGMTCQSCVSSIEGKVRKLQGVVRVKVSLSNQEAVITYQPYLIQPEDLRDHVNDMGFEAAIKSKVAPLSLGPIDIERLQSTNPKRPLSSANQNFNNSETLGHQGSHVVTLQLRIDGMHCKSCVLNIEENIGQLLGVQSIQVSLENKTAQVKYDPSCTSPVALQRAIEALPPGNFKVSLPDGAEGSGTDHRSSSSHSPGSPPRNQVQGTCSTTLIAIAGMTCASCVHSIEGMISQLEGVQQISVSLAEGTATVLYNPSVISPEELRAAIEDMGFEASVVSESCSTNPLGNHSAGNSMVQTTDGTPTSVQEVAPHTGRLPANHAPDILAKSPQSTRAVAPQKCFLQIKGMTCASCVSNIERNLQKEAGVLSVLVALMAGKAEIKYDPEVIQPLEIAQFIQDLGFEAAVMEDYAGSDGNIELTITGMTCASCVHNIESKLTRTNGITYASVALATSKALVKFDPEIIGPRDIIKIIEEIGFHASLAQRNPNAHHLDHKMEIKQWKKSFLCSLVFGIPVMALMIYMLIPSNEPHQSMVLDHNIIPGLSILNLIFFILCTFVQLLGGWYFYVQAYKSLRHRSANMDVLIVLATSIAYVYSLVILVVAVAEKAERSPVTFFDTPPMLFVFIALGRWLEHLAKSKTSEALAKLMSLQATEATVVTLGEDNLIIREEQVPMELVQRGDIVKVVPGGKFPVDGKVLEGNTMADESLITGEAMPVTKKPGSTVIAGSINAHGSVLIKATHVGNDTTLAQIVKLVEEAQMSKAPIQQLADRFSGYFVPFIIIMSTLTLVVWIVIGFIDFGVVQRYFPNPNKHISQTEVIIRFAFQTSITVLCIACPCSLGLATPTAVMVGTGVAAQNGILIKGGKPLEMAHKIKTVMFDKTGTITHGVPRVMRVLLLGDVATLPLRKVLAVVGTAEASSEHPLGVAVTKYCKEELGTETLGYCTDFQAVPGCGIGCKVSNVEGILAHSERPLSAPASHLNEAGSLPAEKDAVPQTFSVLIGNREWLRRNGLTISSDVSDAMTDHEMKGQTAILVAIDGVLCGMIAIADAVKQEAALAVHTLQSMGVDVVLITGDNRKTARAIATQVGINKVFAEVLPSHKVAKVQELQNKGKKVAMVGDGVNDSPALAQADMGVAIGTGTDVAIEAADVVLIRNDLLDVVASIHLSKRTVRRIRINLVLALIYNLVGIPIAAGVFMPIGIVLQPWMGSAAMAASSVSVVLSSLQLKCYKKPDLERYEAQAHGHMKPLTASQVSVHIGMDDRWRDSPRATPWDQVSYVSQVSLSSLTSDKPSRHSAAADDDGDKWSLLLNGRDEEQYI

SEQ ID NO: 8 - protein, 533 aa, Homo sapiens
MSIQVEHPAGGYKKLFETVEELSSPLTAHVTGRIPLWLTGSLLRCGPGLFEVGSEPFYHLFDGQALLHKFDFKEGHVTYHRRFIRTDAYVRAMTEKRIVITEFGTCAFPDPCKNIFSRFFSYFRGVEVTDNALVNVYPVGEDYYACTETNFITKINPETLETIKQVDLCNYVSVNGATAHPHIENDGTVYNIGNCFGKNFSIAYNIVKIPPLQADKEDPISKSEIVVQFPCSDRFKPSYVHSFGLTPNYIVFVETPVKINLFKFLSSWSLWGANYMDCFESNETMGVWLHIADKKRKKYLNNKYRTSPFNLFHHINTYEDNGFLIVDLCCWKGFEFVYNYLYLANLRENWEEVKKNARKAPQPEVRRYVLPLNIDKADTGKNLVTLPNTTATAILCSDETIWLEPEVLFSGPRQAFEFPQINYQKYCGKPYTYAYGLGLNHFVPDRLCKLNVKTKETWVWQEPDSYPSEPIFVSHPDALEEDDGVVLSVVVSPGAGQKPAYLLILNAKDLSEVARAEVEINIPVTFHGLFKKS

SEQ ID NO: 9 - protein, 294 aa, Homo sapiens
MAMSSGGSGGGVPEQEDSVLFRRGTGQSDDSDIWDDTALIKAYDKAVASFKHALKNGDICETSGKPKTTPKRKPAKKNKSQKKNTAASLQQWKVGDKCSAIWSEDGCIYPATIASIDFKRETCVVVYTGYGNREEQNLSDLLSPICEVANNIEQNAQENENESQVSTDESENSRSPGNKSDNIKPKSAPWNSFLPPPPPMPGPRLGPGKPGLKFNGPPPPPPPPPPHLLSCWLPPFPSGPPIIPPPPPICPDSLDDADALGSMLISWYMSGYHTGYYMGFRQNQKEGRCSHSLN

SEQ ID NO: 10 - protein, 1480 aa, Homo sapiens
MQRSPLEKASVVSKLFFSWTRPILRKGYRQRLELSDIYQIPSVDSADNLSEKLEREWDRELASKKNPKLINALRRCFFWRFMFYGIFLYLGEVTKAVQPLLLGRIIASYDPDNKEERSIAIYLGIGLCLLFIVRTLLLHPAIFGLHHIGMQMRIAMFSLIYKKTLKLSSRVLDKISIGQLVSLLSNNLNKFDEGLALAHFVWIAPLQVALLMGLIWELLQASAFCGLGFLIVLALFQAGLGRMMMKYRDQRAGKISERLVITSEMIENIQSVKAYCWEEAMEKMIENLRQTELKLTRKAAYVRYFNSSAFFFSGFFVVFLSVLPYALIKGIILRKIFTTISFCIVLRMAVTRQFPWAVQTWYDSLGAINKIQDFLQKQEYKTLEYNLTTTEVVMENVTAFWEEGFGELFEKAKQNNNNRKTSNGDDSLFFSNFSLLGTPVLKDINFKIERGQLLAVAGSTGAGKTSLLMVIMGELEPSEGKIKHSGRISFCSQFSWIMPGTIKENIIFGVSYDEYRYRSVIKACQLEEDISKFAEKDNIVLGEGGITLSGGQRARISLARAVYKDADLYLLDSPFGYLDVLTEKEIFESCVCKLMANKTRILVTSKMEHLKKADKILILHEGSSYFYGTFSELQNLQPDFSSKLMGCDSFDQFSAERRNSILTETLHRFSLEGDAPVSWTETKKQSFKQTGEFGEKRKNSILNPINSIRKFSIVQKTPLQMNGIEEDSDEPLERRLSLVPDSEQGEAILPRISVISTGPTLQARRRQSVLNLMTHSVNQGQNIHRKTTASTRKVSLAPQANLTELDIYSRRLSQETGLEISEEINEEDLKECFFDDMESIPAVTTWNTYLRYITVHKSLIFVLIWCLVIFLAEVAASLVVLWLLGNTPLQDKGNSTHSRNNSYAVIITSTSSYYVFYIYVGVADTLLAMGFFRGLPLVHTLITVSKILHHKMLHSVLQAPMSTLNTLKAGGILNRFSKDIAILDDLLPLTIFDFIQLLLIVIGAIAVVAVLQPYIFVATVPVIVAFIMLRAYFLQTSQQLKQLESEGRSPIFTHLVTSLKGLWTLRAFGRQPYFETLFHKALNLHTANWFLYLSTLRWFQMRIEMIFVIFFIAVTFISILTTGEGEGRVGIILTLAMNIMSTLQWAVNSSIDVDSLMRSVSRVFKFIDMPTEGKPTKSTKPYKNGQLSKVMIIENSHVKKDDIWPSGGQMTVKDLTAKYTEGGNAILENISFSISPGQRVGLLGRTGSGKSTLLSAFLRLLNTEGEIQIDGVSWDSITLQQWRKAFGVIPQKVFIFSGTFRKNLDPYEQWSDQEIWKVADEVGLRSVIEQFPGKLDFVLVDGGCVLSHGHKQLMCLARSVLSKAKILLLDEPSAHLDPVTYQIIRRTLKQAFADCTVILCEHRIEAMLECQQFLVIEENKVRQYDSIQKLLNERSLFRQAISPSDRVKLFPHRNSSKCKSKPQIAALKEETEEEVQDTRL

SEQ ID NO: 11 - protein, 144 aa, Homo sapiens
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 12 - protein, 536 aa, Corynebacterium diphtheriae
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 13 - protein, 376 aa, Human alphaherpesvirus 1
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 14 - protein, 1255 aa, Severe acute respiratory syndrome-related coronavirus
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 15 - protein, 565 aa, Alphainfluenzavirus influenzae
  source 1..565: note = A/WSN/1933(H1N1)
MKAFVLVLLYAFVATDADTICIGYHANNSTDTVDTIFEKNVAVTHSVNLLEDRHNGKLCKLKGIAPLQLGKCNITGWLLGNPECDSLLPARSWSYIVETPNSENGACYPGDFIDYEELREQLSSVSSLERFEIFPKESSWPNHTFNGVTVSCSHRGKSSFYRNLLWLTKKGDSYPKLTNSYVNNKGKEVLVLWGVHHPSSSDEQQSLYSNGNAYVSVASSNYNRRFTPEIAARPKVKDQHGRMNYYWTLLEPGDTIIFEATGNLIAPWYAFALSRGFESGIITSNASMHECNTKCQTPQGSINSNLPFQNIHPVTIGECPKYVRSTKLRMVTGLRNIPSIQYRGLFGAIAGFIEGGWTGMIDGWYGYHHQNEQGSGYAADQKSTQNAINRITNKVNSVIEKMNTQFTAVGKEFNNLEKRMENLNKKVDDGFLDIWTYNAELLVLLENERTLDFHDLNVKNLYEKVKSQLKNNAKEIGNGCFEFYHKCDNECMESVRNGTYDYPKYSEESKLNREKIDGVKLESMGVYQILAIYSTVASSLVLLVSLGAISFWMCSNGSLQCRICI

SEQ ID NO: 16 - protein, 564 aa, Alphainfluenzavirus influenzae
  source 1..564: note = strain A/Chicken/Scotland/1959 H5N1
MERIVLLLAIVSLVKSDQICIGYHANKSTKQVDTIMEKNVTVTHAQDILERTHNGKLCSLNGVKPLILRDCSVAGWLLGNPMCDEFLNVPEWSYIVEKDNPINSLCYPGDFNDYEELKHLLSSTNHFEKIQIIPRSSWSNHDASSGVSSACPYIGRSSFFRNVVWLIKKDNAYPTIKRSYNNTNQEDLLILWGIHHPNDAAEQTKLYQNPTTYVSVGTSTLNQRSIPEIATRPKVNGQSGRMEFFWTILKPNDAINFESNGNFIAPEYAYKIVKKGDSAIMKSGLAYGNCDTKCQTPVGAINSSMPFHNIHPHTIGECPKYVKSDRLVLATGLRNVPQRKKRGLFGAIAGFIEGGWQGMVDGWYGYHHSNEQGSGYAADKESTQKAIDGITNKVNSIIDKMNTQFKAVGKEFNNLERRVENLNKKMEDGFLDVWTYNVELLVLMENERTLDFHDSNVKNLYDKVRLQLKDNARELGNGCFEFYHKCDNECMESVRNGTYDYPQYSEEARLNREEISGVKLESMGVYQILSIYSTVASSLALAIMIAGLSFWMCSNGSLQCRICI

SEQ ID NO: 17 - protein, 415 aa, Human mastadenovirus C
  source 1..415: note = serotype 5 (HAdV-5)
MHPVLRQMRPPPQQRQEQEQRQTCRAPSPPPTASGGATSAVDAAADGDYEPPRRRARHYLDLEEGEGLARLGAPSPERYPRVQLKRDTREAYVPRQNLFRDREGEEPEEMRDRKFHAGRELRHGLNRERLLREEDFEPDARTGISPARAHVAAADLVTAYEQTVNQEINFQKSFNNHVRTLVAREEVAIGLMHLWDFVSALEQNPNSKPLMAQLFLIVQHSRDNEAFRDALLNIVEPEGRWLLDLINILQSIVVQERSLSLADKVAAINYSMLSLGKFYARKIYHTPYVPIDKEVKIEGFYMRMALKVLTLSDDLGVYRNERIHKAVSVSRRRELSDRELMHSLQRALAGTGSGDREAESYFDAGADLRWAPSRRALEAAGAGPGLAVAPARAGNVGGVEEYDEDDEYEPEDGEY

SEQ ID NO: 18 - protein, 415 aa, Human mastadenovirus C
  source 1..415: note = serotype 5(HAdV-5)
MHPVLRQMRPPPQQRQEQEQRQTCRAPSPPPTASGGATSAVDAAADGDYEPPRRRARHYLDLEEGEGLARLGAPSPERHPRVQLKRDTREAYVPRQNLFRDREGEEPEEMRDRKFHAGRELRHGLNRERLLREEDFEPDARTGISPARAHVAAADLVTAYEQTVNQEINFQKSFNNHVRTLVAREEVAIGLMHLWDFVSALEQNPNSKPLMAQLFLIVQHSRDNEAFRDALLNIVEPEGRWLLDLINILQSIVVQERSLSLADKVAAINYSMLSLGKFYARKIYHTPYVPIDKEVKIEGFYMRMALKVLTLSDDLGVYRNERIHKAVSVSRRRELSDRELMHSLQRALAGTGSGDREAESYFDAGADLRWAPSRRALEAAGAGPGLAVAPARAGNVGGVEEYDEDDEYEPEDGEY